SEQUENCE LISTING (198 sequences)

SEQ ID NO: 1 - protein, 5 aa, Homo Sapiens
SYWMT

SEQ ID NO: 2 - protein, 15 aa, Homo Sapiens
SIKIKPDGYAASVKG

SEQ ID NO: 3 - protein, 11 aa, Homo Sapiens
LFYQYFARMDY

SEQ ID NO: 4 - protein, 11 aa, Homo Sapiens
RASQDISNYLN

SEQ ID NO: 5 - protein, 7 aa, Homo Sapiens
AASNLQS

SEQ ID NO: 6 - protein, 9 aa, Homo Sapiens
QQYDSYSPT

SEQ ID NO: 7 - protein, 118 aa, Homo Sapiens
EVQLVESGGGLVQPGGSLRLSCAASGFTFSSYWMTWVRQAPGKGLEWVSSIKIKPDGYAASVKGRFTISRDNSKNTLYLQMNSLRAEDTAVYYCARLFYQYFARMDYWGQGTLVTVSS

SEQ ID NO: 8 - protein, 107 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQYDSYSPTFGQGTKVEIK

SEQ ID NO: 9 - protein, 448 aa, Homo Sapiens
EVQLVESGGGLVQPGGSLRLSCAASGFTFSSYWMTWVRQAPGKGLEWVSSIKIKPDGYAASVKGRFTISRDNSKNTLYLQMNSLRAEDTAVYYCARLFYQYFARMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQYDSYSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 354 bp, Homo Sapiens
gaggtgcaattggtggaatctggcggcggactggtgcagcctggcggcagcctgagactgagctgcgccgccagcggcttcaccttcagcagctactggatgacatgggtgcgccaggcccctggcaagggactggaatgggtgtccagcatcaagatcaagcccgacggctacgccgcctccgtgaagggccggttcaccatcagccgggacaacagcaagaacaccctgtacctgcagatgaacagcctgcgggccgaggacaccgccgtgtactactgcgccagactgttctaccagtacttcgcccggatggactactggggccagggcaccctggtgaccgtgagctca

SEQ ID NO: 12 - DNA, 321 bp, Homo Sapiens
gatatccagatgacccagagccccagcagcctgagcgccagcgtgggcgacagagtgaccatcacctgccgggccagccaggacatcagcaactacctgaactggtatcagcagaagcccggcaaggcccccaagctgctgatctacgccgccagcaacctgcagagcggcgtgcccagccggtttagcggcagcggctccggcaccgacttcaccctgaccatcagctccctgcagcccgaggacttcgccacctactactgccagcagtacgacagctacagccccaccttcggccagggcaccaaggtggagatcaag

SEQ ID NO: 13 - DNA, 1404 bp, Homo Sapiens
atgaagcacctgtggttctttctgctgctggtcgccgctcccagatgggtgctgtccgaggtgcaattggtggaatctggcggcggactggtgcagcctggcggcagcctgagactgagctgcgccgccagcggcttcaccttcagcagctactggatgacatgggtgcgccaggcccctggcaagggactggaatgggtgtccagcatcaagatcaagcccgacggctacgccgcctccgtgaagggccggttcaccatcagccgggacaacagcaagaacaccctgtacctgcagatgaacagcctgcgggccgaggacaccgccgtgtactactgcgccagactgttctaccagtacttcgcccggatggactactggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - DNA, 642 bp, Homo Sapiens
gatatccagatgacccagagccccagcagcctgagcgccagcgtgggcgacagagtgaccatcacctgccgggccagccaggacatcagcaactacctgaactggtatcagcagaagcccggcaaggcccccaagctgctgatctacgccgccagcaacctgcagagcggcgtgcccagccggtttagcggcagcggctccggcaccgacttcaccctgaccatcagctccctgcagcccgaggacttcgccacctactactgccagcagtacgacagctacagccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 5 aa, Homo Sapiens
SYWMT

SEQ ID NO: 16 - protein, 15 aa, Homo Sapiens
SIKIKPDGYAASVKG

SEQ ID NO: 17 - protein, 11 aa, Homo Sapiens
LFYQYFARMDY

SEQ ID NO: 18 - protein, 11 aa, Homo Sapiens
RASQDISNYLN

SEQ ID NO: 19 - protein, 7 aa, Homo Sapiens
AASNLQS

SEQ ID NO: 20 - protein, 9 aa, Homo Sapiens
QQHDTFRPT

SEQ ID NO: 21 - protein, 118 aa, Homo Sapiens; identical to SEQ ID NO: 7

SEQ ID NO: 22 - protein, 107 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQHDTFRPTFGQGTKVEIK

SEQ ID NO: 23 - protein, 448 aa, Homo Sapiens; identical to SEQ ID NO: 9

SEQ ID NO: 24 - protein, 214 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQHDTFRPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 354 bp, Homo Sapiens; identical to SEQ ID NO: 11

SEQ ID NO: 26 - DNA, 321 bp, Homo Sapiens
gatatccagatgacccagagccccagcagcctgagcgccagcgtgggcgacagagtgaccatcacctgtcgggccagccaggacatcagcaactacctgaactggtatcagcagaagcccggcaaggcccccaagctgctgatctacgccgccagcaatctgcagagcggcgtgcccagccggtttagcggcagcggctccggcaccgactttaccctgacaatttcctctctgcagcctgaggacttcgccacctactactgccagcagcacgacaccttccggcccaccttcggccagggcaccaaggtggagatcaag

SEQ ID NO: 27 - DNA, 1404 bp, Homo Sapiens; identical to SEQ ID NO: 13

SEQ ID NO: 28 - DNA, 642 bp, Homo Sapiens
gatatccagatgacccagagccccagcagcctgagcgccagcgtgggcgacagagtgaccatcacctgtcgggccagccaggacatcagcaactacctgaactggtatcagcagaagcccggcaaggcccccaagctgctgatctacgccgccagcaatctgcagagcggcgtgcccagccggtttagcggcagcggctccggcaccgactttaccctgacaatttcctctctgcagcctgaggacttcgccacctactactgccagcagcacgacaccttccggcccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - protein, 5 aa, Homo Sapiens
SYWMT

SEQ ID NO: 30 - protein, 15 aa, Homo Sapiens
SIKIKPDGYAASVKG

SEQ ID NO: 31 - protein, 11 aa, Homo Sapiens
LFYQYFARMDY

SEQ ID NO: 32 - protein, 11 aa, Homo Sapiens
RASQDISNYLN

SEQ ID NO: 33 - protein, 7 aa, Homo Sapiens
AASNLQS

SEQ ID NO: 34 - protein, 9 aa, Homo Sapiens
QQWDSFSPT

SEQ ID NO: 35 - protein, 118 aa, Homo Sapiens; identical to SEQ ID NO: 7

SEQ ID NO: 36 - protein, 107 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQWDSFSPTFGQGTKVEIK

SEQ ID NO: 37 - protein, 448 aa, Homo Sapiens; identical to SEQ ID NO: 9

SEQ ID NO: 38 - protein, 214 aa, Homo Sapiens
DIQMTQSPSSLSASVGDRVTITCRASQDISNYLNWYQQKPGKAPKLLIYAASNLQSGVPSRFSGSGSGTDFTLTISSLQPEDFATYYCQQWDSFSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 354 bp, Homo Sapiens; identical to SEQ ID NO: 11

SEQ ID NO: 40 - DNA, 321 bp, Homo Sapiens
gatatccagatgacccagagccccagcagcctgagcgccagcgtgggcgacagagtgaccatcacctgtcgggccagccaggacatcagcaactacctgaactggtatcagcagaagcccggcaaggcccccaagctgctgatctacgccgccagcaatctgcagagcggcgtgcccagccggtttagcggcagcggctccggcaccgactttaccctgacaatctcctctctgcagcctgaggacttcgccacctactactgccagcagtgggacagcttcagccccaccttcggccagggcaccaaggtggagatcaag

SEQ ID NO: 41 - DNA, 1404 bp, Homo Sapiens; identical to SEQ ID NO: 13

SEQ ID NO: 42 - DNA, 642 bp, Homo Sapiens
gatatccagatgacccagagccccagcagcctgagcgccagcgtgggcgacagagtgaccatcacctgtcgggccagccaggacatcagcaactacctgaactggtatcagcagaagcccggcaaggcccccaagctgctgatctacgccgccagcaatctgcagagcggcgtgcccagccggtttagcggcagcggctccggcaccgactttaccctgacaatctcctctctgcagcctgaggacttcgccacctactactgccagcagtgggacagcttcagccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 43 - protein, 5 aa, Homo Sapiens
SYTFS

SEQ ID NO: 44 - protein, 17 aa, Homo Sapiens
NILPIFGDANYAQKFQG

SEQ ID NO: 45 - protein, 16 aa, Homo Sapiens
NKGAFYYMSTYPSLDV

SEQ ID NO: 46 - protein, 11 aa, Homo Sapiens
RASQNINYYLN

SEQ ID NO: 47 - protein, 7 aa, Homo Sapiens
DAFSLQS

SEQ ID NO: 48 - protein, 10 aa, Homo Sapiens
QQSWSVPPFT

SEQ ID NO: 49 - protein, 125 aa, Homo Sapiens
EVQLVQSGAEVKKPGSSVKVSCKASGGTFSSYTFSWVRQAPGQGLEWMGNILPIFGDANYAQKFQGRVTITADESTSTAYMELSSLRSEDTAVYYCARNKGAFYYMSTYPSLDVWGQGTLVTVSS

SEQ ID NO: 50 - protein, 108 aa, Homo Sapiens
DIQMTQSPSSLSASVGDRVTITCRASQNINYYLNWYQQKPGKAPKLLIYDAFSLQSGVPSRFSGSGSGTDFTLTISSLQPEDFATYYCQQSWSVPPFTFGQGTKVEIK

SEQ ID NO: 51 - protein, 455 aa, Homo Sapiens
EVQLVQSGAEVKKPGSSVKVSCKASGGTFSSYTFSWVRQAPGQGLEWMGNILPIFGDANYAQKFQGRVTITADESTSTAYMELSSLRSEDTAVYYCARNKGAFYYMSTYPSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 215 aa, Homo Sapiens
DIQMTQSPSSLSASVGDRVTITCRASQNINYYLNWYQQKPGKAPKLLIYDAFSLQSGVPSRFSGSGSGTDFTLTISSLQPEDFATYYCQQSWSVPPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 375 bp, Homo Sapiens
gaggtgcaattggtgcagagcggagccgaagtgaagaagcccggcagcagcgtcaaggtgtcctgcaaggccagcggcggcaccttcagcagctacaccttcagctgggtgcgccaggccccaggacagggcctggaatggatgggcaacatcctgcccatcttcggcgacgccaactacgcccagaagttccagggcagagtcaccatcaccgccgacgagagcaccagcaccgcctacatggaactgagcagcctgcggagcgaggacaccgccgtgtactactgcgcccggaacaagggcgccttctactacatgagcacctaccccagcctggacgtgtggggccagggcaccctggtgaccgtgagctca

SEQ ID NO: 54 - DNA, 324 bp, Homo Sapiens
gatatccagatgacccagagccccagcagcctgagcgccagcgtgggcgacagagtgaccatcacctgtcgggccagccagaacatcaactactacctgaactggtatcagcagaagcccggcaaggcccccaagctgctgatctacgacgccttcagcctgcagagcggcgtgcccagccggtttagcggcagcggctccggcaccgactttaccctgacaatttcctctctgcagcctgaggacttcgccacctactactgccagcagtcttggagcgtgccccccttcaccttcggccagggcaccaaggtggagatcaag

SEQ ID NO: 55 - DNA, 1365 bp, Homo Sapiens
gaggtgcaattggtgcagagcggagccgaagtgaagaagcccggcagcagcgtcaaggtgtcctgcaaggccagcggcggcaccttcagcagctacaccttcagctgggtgcgccaggccccaggacagggcctggaatggatgggcaacatcctgcccatcttcggcgacgccaactacgcccagaagttccagggcagagtcaccatcaccgccgacgagagcaccagcaccgcctacatggaactgagcagcctgcggagcgaggacaccgccgtgtactactgcgcccggaacaagggcgccttctactacatgagcacctaccccagcctggacgtgtggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 56 - DNA, 645 bp, Homo Sapiens
gatatccagatgacccagagccccagcagcctgagcgccagcgtgggcgacagagtgaccatcacctgtcgggccagccagaacatcaactactacctgaactggtatcagcagaagcccggcaaggcccccaagctgctgatctacgacgccttcagcctgcagagcggcgtgcccagccggtttagcggcagcggctccggcaccgactttaccctgacaatttcctctctgcagcctgaggacttcgccacctactactgccagcagtcttggagcgtgccccccttcaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 57 - protein, 5 aa, Homo Sapiens
SYTFS

SEQ ID NO: 58 - protein, 17 aa, Homo Sapiens
NILPIFGDANYAQKFQG

SEQ ID NO: 59 - protein, 16 aa, Homo Sapiens
NKGAFYYMSTYPSLDV

SEQ ID NO: 60 - protein, 11 aa, Homo Sapiens
RASQNINYYLN

SEQ ID NO: 61 - protein, 7 aa, Homo Sapiens
DAFSLQS

SEQ ID NO: 62 - protein, 10 aa, Homo Sapiens
QQSIAVPPFT

SEQ ID NO: 63 - protein, 125 aa, Homo Sapiens; identical to SEQ ID NO: 49

SEQ ID NO: 64 - protein, 108 aa, Homo Sapiens
DIQMTQSPSSLSASVGDRVTITCRASQNINYYLNWYQQKPGKAPKLLIYDAFSLQSGVPSRFSGSGSGTDFTLTISSLQPEDFATYYCQQSIAVPPFTFGQGTKVEIK

SEQ ID NO: 65 - protein, 455 aa, Homo Sapiens; identical to SEQ ID NO: 51

SEQ ID NO: 66 - protein, 215 aa, Homo Sapiens
DIQMTQSPSSLSASVGDRVTITCRASQNINYYLNWYQQKPGKAPKLLIYDAFSLQSGVPSRFSGSGSGTDFTLTISSLQPEDFATYYCQQSIAVPPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 375 bp, Homo Sapiens; identical to SEQ ID NO: 53

SEQ ID NO: 68 - DNA, 324 bp, Homo Sapiens
gatatccagatgacccagagccccagcagcctgagcgccagcgtgggcgacagagtgaccatcacctgtcgggccagccagaacatcaactactacctgaactggtatcagcagaagcccggcaaggcccccaagctgctgatctacgacgccttcagcctgcagagcggcgtgcccagccggtttagcggcagcggctccggcaccgactttaccctgacaatttcctctctgcagcctgaggacttcgccacctactactgccagcagagcattgccgtgccccccttcaccttcggccagggcaccaaggtggagatcaag

SEQ ID NO: 69 - DNA, 1365 bp, Homo Sapiens; identical to SEQ ID NO: 55

SEQ ID NO: 70 - DNA, 645 bp, Homo Sapiens
gatatccagatgacccagagccccagcagcctgagcgccagcgtgggcgacagagtgaccatcacctgtcgggccagccagaacatcaactactacctgaactggtatcagcagaagcccggcaaggcccccaagctgctgatctacgacgccttcagcctgcagagcggcgtgcccagccggtttagcggcagcggctccggcaccgactttaccctgacaatttcctctctgcagcctgaggacttcgccacctactactgccagcagagcattgccgtgccccccttcaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - protein, 5 aa, Homo Sapiens
SYSMH

SEQ ID NO: 72 - protein, 17 aa, Homo Sapiens
LINPYNGNTHYAQKFQG

SEQ ID NO: 73 - protein, 6 aa, Homo Sapiens
MLRFDV

SEQ ID NO: 74 - protein, 14 aa, Homo Sapiens
TGTSSDGGGYNYVS

SEQ ID NO: 75 - protein, 7 aa, Homo Sapiens
GVSNRPS

SEQ ID NO: 76 - protein, 11 aa, Homo Sapiens
QTYTRYSDSPV

SEQ ID NO: 77 - protein, 115 aa, Homo Sapiens
EVQLVQSGAEVKKPGASVKVSCKASGYTFTSYSMHWVRQAPGQGLEWMGLINPYNGNTHYAQKFQGRVTMTRDTSISTAYMELSSLRSEDTAVYYCARMLRFDVWGQGTLVTVSS

SEQ ID NO: 78 - protein, 113 aa, Homo Sapiens
ESALTQPASVSGSPGQSITISCTGTSSDGGGYNYVSWYQQHPGKAPKLMIYGVSNRPSGVSNRFSGSKSGNTASLTISGLQAEDEADYYCQTYTRYSDSPVFGGGTKLTVLGQ

SEQ ID NO: 79 - protein, 445 aa, Homo Sapiens
EVQLVQSGAEVKKPGASVKVSCKASGYTFTSYSMHWVRQAPGQGLEWMGLINPYNGNTHYAQKFQGRVTMTRDTSISTAYMELSSLRSEDTAVYYCARMLRFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 217 aa, Homo Sapiens
ESALTQPASVSGSPGQSITISCTGTSSDGGGYNYVSWYQQHPGKAPKLMIYGVSNRPSGVSNRFSGSKSGNTASLTISGLQAEDEADYYCQTYTRYSDSPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - DNA, 345 bp, Homo Sapiens
gaggtgcagctggtgcagagcggagccgaagtgaagaaaccaggcgcttccgtgaaggtgtcctgcaaggccagcggctacaccttcaccagctacagcatgcactgggtccggcaggctccagggcagggactggaatggatgggcctgatcaacccctacaacggcaacacccactacgcccagaaattccagggcagagtgaccatgacccgggacaccagcatcagcaccgcctacatggaactgagcagcctgcggagcgaggacaccgccgtgtactactgcgcccggatgctgcggttcgacgtgtggggccagggcaccctggtcaccgtcagctca

SEQ ID NO: 82 - DNA, 333 bp, Homo Sapiens
gagagcgccctgacccagcctgccagcgtgtctggcagccctggccagagcatcaccatcagctgcaccggcaccagcagcgacggcggaggctacaactacgtgtcctggtatcagcagcaccccggcaaggcccccaagctgatgatctacggcgtgagcaaccggcccagcggggtgtccaaccggttcagcggcagcaagagcggcaacaccgccagcctgaccatctctgggctgcaggctgaggacgaggccgactactactgccagacctacaccagatacagcgacagccctgtgttcggaggcggaacaaagttaaccgtccta

SEQ ID NO: 83 - DNA, 1335 bp, Homo Sapiens
gaggtgcagctggtgcagagcggagccgaagtgaagaaaccaggcgcttccgtgaaggtgtcctgcaaggccagcggctacaccttcaccagctacagcatgcactgggtccggcaggctccagggcagggactggaatggatgggcctgatcaacccctacaacggcaacacccactacgcccagaaattccagggcagagtgaccatgacccgggacaccagcatcagcaccgcctacatggaactgagcagcctgcggagcgaggacaccgccgtgtactactgcgcccggatgctgcggttcgacgtgtggggccagggcaccctggtcaccgtcagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - DNA, 651 bp, Homo Sapiens
gagagcgccctgacccagcctgccagcgtgtctggcagccctggccagagcatcaccatcagctgcaccggcaccagcagcgacggcggaggctacaactacgtgtcctggtatcagcagcaccccggcaaggcccccaagctgatgatctacggcgtgagcaaccggcccagcggggtgtccaaccggttcagcggcagcaagagcggcaacaccgccagcctgaccatctctgggctgcaggctgaggacgaggccgactactactgccagacctacaccagatacagcgacagccctgtgttcggaggcggaacaaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 85 - protein, 5 aa, Homo Sapiens
GYYIN

SEQ ID NO: 86 - protein, 17 aa, Homo Sapiens
IISPNQGTTGYAQKFQG

SEQ ID NO: 87 - protein, 8 aa, Homo Sapiens
GNYDHLDY

SEQ ID NO: 88 - protein, 11 aa, Homo Sapiens
RASQGISNYLN

SEQ ID NO: 89 - protein, 7 aa, Homo Sapiens
DASTLQS

SEQ ID NO: 90 - protein, 10 aa, Homo Sapiens
QQDWHTLPVT

SEQ ID NO: 91 - protein, 117 aa, Homo Sapiens
EVQLVQSGAEVKKPGASVKVSCKASGYTFTGYYINWVRQAPGQGLEWMGIISPNQGTTGYAQKFQGRVTMTRDTSISTAYMELSSLRSEDTAVYYCARGNYDHLDYWGQGTLVTVSS

SEQ ID NO: 92 - protein, 108 aa, Homo Sapiens
DIQMTQSPSSLSASVGDRVTITCRASQGISNYLNWYQQKPGKAPKLLIYDASTLQSGVPSRFSGSGSGTDFTLTISSLQPEDFATYYCQQDWHTLPVTFGQGTKVEIK

SEQ ID NO: 93 - protein, 447 aa, Homo Sapiens
EVQLVQSGAEVKKPGASVKVSCKASGYTFTGYYINWVRQAPGQGLEWMGIISPNQGTTGYAQKFQGRVTMTRDTSISTAYMELSSLRSEDTAVYYCARGNYDHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 215 aa, Homo Sapiens
DIQMTQSPSSLSASVGDRVTITCRASQGISNYLNWYQQKPGKAPKLLIYDASTLQSGVPSRFSGSGSGTDFTLTISSLQPEDFATYYCQQDWHTLPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 351 bp, Homo Sapiens
gaggtgcagctggtgcagagcggagccgaagtgaagaaaccaggcgcttccgtgaaggtgtcctgcaaggccagcggctacaccttcaccggctactacatcaactgggtccggcaggctccagggcagggactggaatggatgggcatcatcagccccaaccagggcacaaccggctacgcccagaaattccagggcagagtgaccatgacccgggacaccagcatcagcaccgcctacatggaactgagcagcctgcggagcgaggacaccgccgtgtactactgcgccagaggcaactacgaccacctggactactggggccagggcaccctggtcaccgtcagctca

SEQ ID NO: 96 - DNA, 324 bp, Homo Sapiens
gatatccagatgacccagagccccagcagcctgagcgccagcgtgggcgacagagtgaccatcacctgtcgggccagccagggcatcagcaactacctgaactggtatcagcagaagcccggcaaggcccccaagctgctgatctacgacgccagcaccctgcagagcggcgtgcctagcagattctccgggagcggctccggcaccgacttcaccctgaccattagctcactgcagccagaagacttcgccacctactactgccagcaggactggcacaccctgcccgtgaccttcggccagggcaccaaggtggagatcaag

SEQ ID NO: 97 - DNA, 1341 bp, Homo Sapiens
gaggtgcagctggtgcagagcggagccgaagtgaagaaaccaggcgcttccgtgaaggtgtcctgcaaggccagcggctacaccttcaccggctactacatcaactgggtccggcaggctccagggcagggactggaatggatgggcatcatcagccccaaccagggcacaaccggctacgcccagaaattccagggcagagtgaccatgacccgggacaccagcatcagcaccgcctacatggaactgagcagcctgcggagcgaggacaccgccgtgtactactgcgccagaggcaactacgaccacctggactactggggccagggcaccctggtcaccgtcagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - DNA, 645 bp, Homo Sapiens
gatatccagatgacccagagccccagcagcctgagcgccagcgtgggcgacagagtgaccatcacctgtcgggccagccagggcatcagcaactacctgaactggtatcagcagaagcccggcaaggcccccaagctgctgatctacgacgccagcaccctgcagagcggcgtgcctagcagattctccgggagcggctccggcaccgacttcaccctgaccattagctcactgcagccagaagacttcgccacctactactgccagcaggactggcacaccctgcccgtgaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 99 - protein, 5 aa, Homo Sapiens
SYWMT

SEQ ID NO: 100 - protein, 15 aa, Homo Sapiens
SIKIKPDGYAASVKG

SEQ ID NO: 101 - protein, 11 aa, Homo Sapiens
LFYQYFARMDY

SEQ ID NO: 102 - protein, 11 aa, Homo Sapiens
RASQDISNYLN

SEQ ID NO: 103 - protein, 7 aa, Homo Sapiens
AASNLQS

SEQ ID NO: 104 - protein, 9 aa, Homo Sapiens
QQYDSYSPT

SEQ ID NO: 105 - protein, 118 aa, Homo Sapiens
QVQLVESGGGLVQPGGSLRLSCAASGFTFSSYWMTWVRQAPGKGLEWVSSIKIKPDGYAASVKGRFTISRDNSKNTLYLQMNSLRAEDTAVYYCARLFYQYFARMDYWGQGTLVTVSS

SEQ ID NO: 106 - protein, 107 aa, Homo Sapiens; identical to SEQ ID NO: 8

SEQ ID NO: 107 - protein, 220 aa, Homo Sapiens
QVQLVESGGGLVQPGGSLRLSCAASGFTFSSYWMTWVRQAPGKGLEWVSSIKIKPDGYAASVKGRFTISRDNSKNTLYLQMNSLRAEDTAVYYCARLFYQYFARMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 108 - protein, 214 aa, Homo Sapiens; identical to SEQ ID NO: 10

SEQ ID NO: 109 - DNA, 354 bp, Homo Sapiens
caggtgcaattggtggaaagcggcggcggcctggtgcaaccgggcggcagcctgcgtctgagctgcgcggcctccggatttaccttttcttcttattggatgacttgggtgcgccaagcccctgggaagggtctcgagtgggtgagctctattaagattaagcctgatggttatgctgcttctgttaagggtcgttttaccatttcacgtgataattcgaaaaacaccctgtatctgcaaatgaacagcctgcgtgcggaagatacggccgtgtattattgcgcgcgtcttttttatcagtattttgctcgtatggattattggggccaaggcaccctggtgacggttagctca

SEQ ID NO: 110 - DNA, 321 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcggtttattattgccagcagtatgattcttattctcctacctttggccagggtacgaaagttgaaattaaa

SEQ ID NO: 111 - DNA, 660 bp, Homo Sapiens
caggtgcaattggtggaaagcggcggcggcctggtgcaaccgggcggcagcctgcgtctgagctgcgcggcctccggatttaccttttcttcttattggatgacttgggtgcgccaagcccctgggaagggtctcgagtgggtgagctctattaagattaagcctgatggttatgctgcttctgttaagggtcgttttaccatttcacgtgataattcgaaaaacaccctgtatctgcaaatgaacagcctgcgtgcggaagatacggccgtgtattattgcgcgcgtcttttttatcagtattttgctcgtatggattattggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 112 - DNA, 642 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcggtttattattgccagcagtatgattcttattctcctacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 113 - protein, 5 aa, Homo Sapiens
SYWMT

SEQ ID NO: 114 - protein, 15 aa, Homo Sapiens
SIKIKPDGYAASVKG

SEQ ID NO: 115 - protein, 11 aa, Homo Sapiens
LFYQYFARMDY

SEQ ID NO: 116 - protein, 11 aa, Homo Sapiens
RASQDISNYLN

SEQ ID NO: 117 - protein, 7 aa, Homo Sapiens
AASNLQS

SEQ ID NO: 118 - protein, 9 aa, Homo Sapiens
QQHDTFRPT

SEQ ID NO: 119 - protein, 118 aa, Homo Sapiens; identical to SEQ ID NO: 105

SEQ ID NO: 120 - protein, 107 aa, Homo Sapiens; identical to SEQ ID NO: 22

SEQ ID NO: 121 - protein, 220 aa, Homo Sapiens; identical to SEQ ID NO: 107

SEQ ID NO: 122 - protein, 214 aa, Homo Sapiens; identical to SEQ ID NO: 24

SEQ ID NO: 123 - DNA, 354 bp, Homo Sapiens; identical to SEQ ID NO: 109

SEQ ID NO: 124 - DNA, 321 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcgacctattattgccagcagcatgatacttttcgtcctacctttggccagggtacgaaagttgaaattaaa

SEQ ID NO: 125 - DNA, 660 bp, Homo Sapiens; identical to SEQ ID NO: 111

SEQ ID NO: 126 - DNA, 642 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcgacctattattgccagcagcatgatacttttcgtcctacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 127 - protein, 5 aa, Homo Sapiens
SYWMT

SEQ ID NO: 128 - protein, 15 aa, Homo Sapiens
SIKIKPDGYAASVKG

SEQ ID NO: 129 - protein, 11 aa, Homo Sapiens
LFYQYFARMDY

SEQ ID NO: 130 - protein, 11 aa, Homo Sapiens
RASQDISNYLN

SEQ ID NO: 131 - protein, 7 aa, Homo Sapiens
AASNLQS

SEQ ID NO: 132 - protein, 9 aa, Homo Sapiens
QQWDSFSPT

SEQ ID NO: 133 - protein, 118 aa, Homo Sapiens; identical to SEQ ID NO: 105

SEQ ID NO: 134 - protein, 107 aa, Homo Sapiens; identical to SEQ ID NO: 36

SEQ ID NO: 135 - protein, 220 aa, Homo Sapiens; identical to SEQ ID NO: 107

SEQ ID NO: 136 - protein, 214 aa, Homo Sapiens; identical to SEQ ID NO: 38

SEQ ID NO: 137 - DNA, 354 bp, Homo Sapiens; identical to SEQ ID NO: 109

SEQ ID NO: 138 - DNA, 321 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcgacctattattgccagcagtgggattctttttctcctacctttggccagggtacgaaagttgaaattaaa

SEQ ID NO: 139 - DNA, 660 bp, Homo Sapiens; identical to SEQ ID NO: 111

SEQ ID NO: 140 - DNA, 642 bp, Homo Sapiens
gatatccagatgacccagagcccgtctagcctgagcgcgagcgtgggtgatcgtgtgaccattacctgcagagcgagccaggatatttctaattatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcgacctattattgccagcagtgggattctttttctcctacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 141 - protein, 5 aa, Homo Sapiens
SYTFS

SEQ ID NO: 142 - protein, 17 aa, Homo Sapiens
NILPIFGDANYAQKFQG

SEQ ID NO: 143 - protein, 16 aa, Homo Sapiens
NKGAFYYMSTYPSLDV

SEQ ID NO: 144 - protein, 11 aa, Homo Sapiens
RASQNINYYLN

SEQ ID NO: 145 - protein, 7 aa, Homo Sapiens
DAFSLQS

SEQ ID NO: 146 - protein, 10 aa, Homo Sapiens
QQSWSVPPFT

SEQ ID NO: 147 - protein, 125 aa, Homo Sapiens
QVQLVQSGAEVKKPGSSVKVSCKASGGTFSSYTFSWVRQAPGQGLEWMGNILPIFGDANYAQKFQGRVTITADESTSTAYMELSSLRSEDTAVYYCARNKGAFYYMSTYPSLDVWGQGTLVTVSS

SEQ ID NO: 148 - protein, 108 aa, Homo Sapiens; identical to SEQ ID NO: 50

SEQ ID NO: 149 - protein, 227 aa, Homo Sapiens
QVQLVQSGAEVKKPGSSVKVSCKASGGTFSSYTFSWVRQAPGQGLEWMGNILPIFGDANYAQKFQGRVTITADESTSTAYMELSSLRSEDTAVYYCARNKGAFYYMSTYPSLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 150 - protein, 215 aa, Homo Sapiens; identical to SEQ ID NO: 52

SEQ ID NO: 151 - DNA, 375 bp, Homo Sapiens
caggtgcaattggttcagtctggcgcggaagtgaaaaaaccgggcagcagcgtgaaagtgagctgcaaagcctccggaggcactttttcttcttatactttttcttgggtgcgccaagcccctgggcagggtctcgagtggatgggcaatatccttccgatttttggcgatgcgaattacgcgcagaagtttcagggccgggtgaccattaccgcggatgaaagcaccagcaccgcgtatatggaactgagcagcctgcgtagcgaagatacggccgtgtattattgcgcgcgtaataagggtgctttttattatatgtctacttatccttctcttgatgtttggggccaaggcaccctggtgacggttagctca

SEQ ID NO: 152 - DNA, 324 bp, Homo Sapiens
gatatccagatgacccagagcccgtctagcctgagcgcgagcgtgggtgatcgtgtgaccattacctgcagagcgagccagaatattaattattatctgaattggtaccagcagaaaccaggtaaagcaccgaaactattaatttatgatgctttttctttgcaaagcggggtcccgtcccgttttagcggctctggatccggcactgattttaccctgaccattagcagcctgcaacctgaagactttgcgacctattattgccagcagtcttggtctgttcctccttttacctttggccagggtacgaaagttgaaattaaa

SEQ ID NO: 153 - DNA, 681 bp, Homo Sapiens
caggtgcaattggttcagtctggcgcggaagtgaaaaaaccgggcagcagcgtgaaagtgagctgcaaagcctccggaggcactttttcttcttatactttttcttgggtgcgccaagcccctgggcagggtctcgagtggatgggcaatatccttccgatttttggcgatgcgaattacgcgcagaagtttcagggccgggtgaccattaccgcggatgaaagcaccagcaccgcgtatatggaactgagcagcctgcgtagcgaagatacggccgtgtattattgcgcgcgtaataagggtgctttttattatatgtctacttatccttctcttgatgtttggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 154 - DNA, 645 bp, Homo Sapiens
gatatccagatgacccagagcccgtctagcctgagcgcgagcgtgggtgatcgtgtgaccattacctgcagagcgagccagaatattaattattatctgaattggtaccagcagaaaccaggtaaagcaccgaaactattaatttatgatgctttttctttgcaaagcggggtcccgtcccgttttagcggctctggatccggcactgattttaccctgaccattagcagcctgcaacctgaagactttgcgacctattattgccagcagtcttggtctgttcctccttttacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 155 - protein, 5 aa, Homo Sapiens
SYTFS

SEQ ID NO: 156 - protein, 17 aa, Homo Sapiens
NILPIFGDANYAQKFQG

SEQ ID NO: 157 - protein, 16 aa, Homo Sapiens
NKGAFYYMSTYPSLDV

SEQ ID NO: 158 - protein, 11 aa, Homo Sapiens
RASQNINYYLN

SEQ ID NO: 159 - protein, 7 aa, Homo Sapiens
DAFSLQS

SEQ ID NO: 160 - protein, 10 aa, Homo Sapiens
QQSIAVPPFT

SEQ ID NO: 161 - protein, 125 aa, Homo Sapiens; identical to SEQ ID NO: 147

SEQ ID NO: 162 - protein, 108 aa, Homo Sapiens; identical to SEQ ID NO: 64

SEQ ID NO: 163 - protein, 227 aa, Homo Sapiens; identical to SEQ ID NO: 149

SEQ ID NO: 164 - protein, 215 aa, Homo Sapiens; identical to SEQ ID NO: 66

SEQ ID NO: 165 - DNA, 375 bp, Homo Sapiens; identical to SEQ ID NO: 151

SEQ ID NO: 166 - DNA, 324 bp, Homo Sapiens
gatatccagatgacccagagcccgtctagcctgagcgcgagcgtgggtgatcgtgtgaccattacctgcagagcgagccagaatattaattattatctgaattggtaccagcagaaaccaggtaaagcaccgaaactattaatttatgatgctttttctttgcaaagcggggtcccgtcccgttttagcggctctggatccggcactgattttaccctgaccattagcagcctgcaacctgaagactttgcgacctattattgccagcagtctattgctgttcctccttttacctttggccagggtacgaaagttgaaattaaa

SEQ ID NO: 167 - DNA, 681 bp, Homo Sapiens; identical to SEQ ID NO: 153

SEQ ID NO: 168 - DNA, 645 bp, Homo Sapiens
gatatccagatgacccagagcccgtctagcctgagcgcgagcgtgggtgatcgtgtgaccattacctgcagagcgagccagaatattaattattatctgaattggtaccagcagaaaccaggtaaagcaccgaaactattaatttatgatgctttttctttgcaaagcggggtcccgtcccgttttagcggctctggatccggcactgattttaccctgaccattagcagcctgcaacctgaagactttgcgacctattattgccagcagtctattgctgttcctccttttacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 169 - protein, 5 aa, Homo Sapiens
SYSMH

SEQ ID NO: 170 - protein, 17 aa, Homo Sapiens
LINPYNGNTHYAQKFQG

SEQ ID NO: 171 - protein, 6 aa, Homo Sapiens
MLRFDV

SEQ ID NO: 172 - protein, 14 aa, Homo Sapiens
TGTSSDGGGYNYVS

SEQ ID NO: 173 - protein, 7 aa, Homo Sapiens
GVSNRPS

SEQ ID NO: 174 - protein, 11 aa, Homo Sapiens
QTYTRYSDSPV

SEQ ID NO: 175 - protein, 115 aa, Homo Sapiens
QVQLVQSGAEVKKPGASVKVSCKASGYTFTSYSMHWVRQAPGQGLEWMGLINPYNGNTHYAQKFQGRVTMTRDTSISTAYMELSSLRSEDTAVYYCARMLRFDVWGQGTLVTVSS

SEQ ID NO: 176 - protein, 111 aa, Homo Sapiens
DIALTQPASVSGSPGQSITISCTGTSSDGGGYNYVSWYQQHPGKAPKLMIYGVSNRPSGVSNRFSGSKSGNTASLTISGLQAEDEADYYCQTYTRYSDSPVFGGGTKLTVL

SEQ ID NO: 177 - protein, 217 aa, Homo Sapiens
QVQLVQSGAEVKKPGASVKVSCKASGYTFTSYSMHWVRQAPGQGLEWMGLINPYNGNTHYAQKFQGRVTMTRDTSISTAYMELSSLRSEDTAVYYCARMLRFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 178 - protein, 216 aa, Homo Sapiens
DIALTQPASVSGSPGQSITISCTGTSSDGGGYNYVSWYQQHPGKAPKLMIYGVSNRPSGVSNRFSGSKSGNTASLTISGLQAEDEADYYCQTYTRYSDSPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 179 - DNA, 345 bp, Homo Sapiens
caggtgcaattggttcagagcggcgcggaagtgaaaaaaccgggcgcgagcgtgaaagtgagctgcaaagcctccggatatacctttacttcttattctatgcattgggtccgccaagcccctgggcagggtctcgagtggatgggccttatcaatccgtataatggcaatacgcattacgcgcagaagtttcagggccgggtgaccatgacccgtgataccagcattagcaccgcgtatatggaactgagcagcctgcgtagcgaagatacggccgtgtattattgcgcgcgtatgcttcgttttgatgtttggggccaaggcaccctggtgacggttagctca

SEQ ID NO: 180 - DNA, 333 bp, Homo Sapiens
gatatcgcactgacccagccagcttcagtgagcggctcaccaggtcagagcattaccatctcgtgtacgggtactagcagcgatggtggtggttataattatgtgtcttggtaccagcagcatcccgggaaggcgccgaaacttatgatttatggtgtttctaatcgtccctcaggcgtgagcaaccgttttagcggatccaaaagcggcaacaccgcgagcctgaccattagcggcctgcaagcggaagacgaagcggattattattgccagacttatactcgttattctgattctcctgtgtttggcggcggcacgaagttaaccgttctt

SEQ ID NO: 181 - DNA, 651 bp, Homo Sapiens
caggtgcaattggttcagagcggcgcggaagtgaaaaaaccgggcgcgagcgtgaaagtgagctgcaaagcctccggatatacctttacttcttattctatgcattgggtccgccaagcccctgggcagggtctcgagtggatgggccttatcaatccgtataatggcaatacgcattacgcgcagaagtttcagggccgggtgaccatgacccgtgataccagcattagcaccgcgtatatggaactgagcagcctgcgtagcgaagatacggccgtgtattattgcgcgcgtatgcttcgttttgatgtttggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 182 - DNA, 648 bp, Homo Sapiens
gatatcgcactgacccagccagcttcagtgagcggctcaccaggtcagagcattaccatctcgtgtacgggtactagcagcgatggtggtggttataattatgtgtcttggtaccagcagcatcccgggaaggcgccgaaacttatgatttatggtgtttctaatcgtccctcaggcgtgagcaaccgttttagcggatccaaaagcggcaacaccgcgagcctgaccattagcggcctgcaagcggaagacgaagcggattattattgccagacttatactcgttattctgattctcctgtgtttggcggcggcacgaagttaaccgttcttggccagccgaaagccgcaccgagtgtgacgctgtttccgccgagcagcgaagaattgcaggcgaacaaagcgaccctggtgtgcctgattagcgacttttatccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaggggagcaccgtggaaaaaaccgttgcgccgactgaggcc

SEQ ID NO: 183 - protein, 5 aa, Homo Sapiens
GYYIN

SEQ ID NO: 184 - protein, 17 aa, Homo Sapiens
IISPNQGTTGYAQKFQG

SEQ ID NO: 185 - protein, 8 aa, Homo Sapiens
GNYDHLDY

SEQ ID NO: 186 - protein, 11 aa, Homo Sapiens
RASQGISNYLN

SEQ ID NO: 187 - protein, 7 aa, Homo Sapiens
DASTLQS

SEQ ID NO: 188 - protein, 10 aa, Homo Sapiens
QQDWHTLPVT

SEQ ID NO: 189 - protein, 117 aa, Homo Sapiens
QVQLVQSGAEVKKPGASVKVSCKASGYTFTGYYINWVRQAPGQGLEWMGIISPNQGTTGYAQKFQGRVTMTRDTSISTAYMELSSLRSEDTAVYYCARGNYDHLDYWGQGTLVTVSS

SEQ ID NO: 190 - protein, 108 aa, Homo Sapiens; identical to SEQ ID NO: 92

SEQ ID NO: 191 - protein, 219 aa, Homo Sapiens
QVQLVQSGAEVKKPGASVKVSCKASGYTFTGYYINWVRQAPGQGLEWMGIISPNQGTTGYAQKFQGRVTMTRDTSISTAYMELSSLRSEDTAVYYCARGNYDHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 192 - protein, 215 aa, Homo Sapiens; identical to SEQ ID NO: 94

SEQ ID NO: 193 - DNA, 351 bp, Homo Sapiens
caggtgcaattggttcagagcggcgcggaagtgaaaaaaccgggcgcgagcgtgaaagtgagctgcaaagcctccggatatacctttactggttattatattaattgggtccgccaagcccctgggcagggtctcgagtggatgggcattatctctccgaatcagggcactacgggttacgcgcagaagtttcagggccgggtgaccatgacccgtgataccagcattagcaccgcgtatatggaactgagcagcctgcgtagcgaagatacggccgtgtattattgcgcgcgtggtaattatgatcatcttgattattggggccaaggcaccctggtgacggttagctca

SEQ ID NO: 194 - DNA, 324 bp, Homo Sapiens
gatatccagatgacccagagcccgtctagcctgagcgcgagcgtgggtgatcgtgtgaccattacctgcagagcgagccagggtatttctaattatctgaattggtaccagcagaaaccaggtaaagcaccgaaactattaatttatgatgcttctactttgcaaagcggggtcccgtcccgttttagcggctctggatccggcactgattttaccctgaccattagcagcctgcaacctgaagactttgcgacttattattgccagcaggattggcatactcttcctgttacctttggccagggtacgaaagttgaaattaaa

SEQ ID NO: 195 - DNA, 657 bp, Homo Sapiens
caggtgcaattggttcagagcggcgcggaagtgaaaaaaccgggcgcgagcgtgaaagtgagctgcaaagcctccggatatacctttactggttattatattaattgggtccgccaagcccctgggcagggtctcgagtggatgggcattatctctccgaatcagggcactacgggttacgcgcagaagtttcagggccgggtgaccatgacccgtgataccagcattagcaccgcgtatatggaactgagcagcctgcgtagcgaagatacggccgtgtattattgcgcgcgtggtaattatgatcatcttgattattggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 196 - DNA, 645 bp, Homo Sapiens
gatatccagatgacccagagcccgtctagcctgagcgcgagcgtgggtgatcgtgtgaccattacctgcagagcgagccagggtatttctaattatctgaattggtaccagcagaaaccaggtaaagcaccgaaactattaatttatgatgcttctactttgcaaagcggggtcccgtcccgttttagcggctctggatccggcactgattttaccctgaccattagcagcctgcaacctgaagactttgcgacttattattgccagcaggattggcatactcttcctgttacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 197 - protein, 645 aa, Homo sapiens
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 198 - protein, 915 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

(19) United States
(12) Patent Application Publication
(10) Pub. No.: US 2010/0291106 A1
(43) Pub. Date: Nov. 18, 2010

(54) COMPOSITIONS AND METHODS FOR ANTIBODIES TARGETING COMPLEMENT PROTEIN C3B

(75) Inventors: Bijan Etemad-Gilbertson, Jamaica Plain, MA (US); Braydon Charles Guild, Concord, MA (US); Yong-In Kim, Westborough, MA (US); Ingo Klagge, Neuried (DE); Alexandra Kraus, Munchen (DE); Michael Roguska, Ashland, MA (US); Igor Splawski, Winchester, MA (US); Kehao Zhao, Shanghai (CN)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG

(21) Appl. No.: 12/773,246

(22) Filed: May 4, 2010

Related U.S. Application Data
(60) Provisional application No. 61/175,860, filed on May 6, 2009.

(51) Int. Cl. A61K 39/395; C07K 16/18; C07H 21/04; C12N 15/63; C12N 5/071; A61P 27/02

(52) U.S. Cl. 424/172.1; 530/389.1; 530/388.2; 530/387.3; 536/23.53; 435/320.1; 435/332

(57) ABSTRACT

The present invention relates to antibodies and antigen binding fragments thereof that bind to both human and cynomolgus complement protein C3b, as well as compositions and methods of use thereof.

### RELATED APPLICATIONS

This application claims priority to U.S. Application Ser. No. 61/175,860 filed May 6, 2009, the contents of which are incorporated herein by reference in its entirety.

### BACKGROUND OF THE INVENTION

Age related macular degeneration (AMD) is a progressive disease and a leading cause of vision loss and blindness in Americans aged 65 and older. AMD primarily affects the macula; a part of the retina responsible for high visual acuity needed to read or drive. The majority of AMD patients suffer from an early stage of the disease which is characterized by the presence of extracellular retinal deposits called drusen. Drusen are extracellular retinal deposits of cell debris, inflammatory mediators, and extracellular matrix components. The late stages of AMD manifest as a dry or wet form, both are associated with vision loss. Dry AMD, also known as geographic atrophy, appears on opthalmoscopic examination as clearly demarcated regions corresponding to local areas of retinal pigmented epithelium (RPE) loss. Wet AMD is associated with neo-vascularization of the choriod, causing a loss of integrity in Bruch's membrane and vessel growth in the retina, where they can often hemorrhage. This leakage causes permanent damage to retinal cells which die off and create blind spots in the central vision.

The innate human system is composed of the complement pathway. The complement pathway serves to defend against pyogenic bacterial infection bridging innate and adaptive immunity; and disposing of products of immune complexes and inflammatory injury. The complement is a system of more than 30 proteins involved in cascade reactions in plasma and cell surfaces. The complement system and its complement components are involved in various immune processes. For example, complement C5b-9 complex, also termed the terminal complex or the membrane attack complex (MAC), plays an important role in cell death by inducing membrane permeability damages.

Recent work has demonstrated that complement components C3 and C5 are principal constituents of drusen in patients with AMD. Mulling, R. F. et al. (2000) FASEB J 14, 835-46 Their presence as well as that of the membrane attack complex (MAC) C5b-9 and other acute phase reactant proteins in RPE cells overlying drusen has been speculated to be involved in the process that can trigger complement activation and formation of MAC. Johnson, L et al. (2001) Exp Eye Res 73, 887-896. Thus, there is growing evidence that complement components are more than mere mediators of innate immunity.

Nutritional intervention has been prescribed to inhibit progression of dry AMD to wet AMD. At present the only FDA approved treatments for wet AMD include photodynamic therapy (PDT), an anti-VEGF aptamer, such as pegaptanib, and anti-VEGF antibodies, ranibizumab. These drugs or therapies are typically administered to patients who have already suffered substantial vision loss.

There remains a need to develop an effective treatment for AMD to replace or supplement current treatments. Particularly, there is a need for treatments which can provide early detection, prevention or restoration of vision loss.

### SUMMARY OF THE INVENTION

The present invention relates to an isolated antibody or antigen binding fragment thereof that specifically binds to a human or cynomolgus complement C3b protein, wherein said antibody binds to human C3b with a KD of less than or equal to 100 pM and cynomolgus C3b with a KD of less than or equal to 200 pM. For example, the antibodies or antigen binding fragments described herein may bind to human C3b with a KD of less than or equal to 90 pM, less than or equal to 80 pM, less than or equal to 70 pM, less than or equal to 60 pM, less than or equal to 50 pM, less than or equal to 40 pM, less than or equal to 30 pM, and preferably as high as less than or equal to 20, 19, 18, 17, 16, 15, 14, 13, 12, or 11 pM. It is preferred that the antibody or antigen binding fragment thereof binds to human C3b with a Kd of less than or equal to 10 pM. For example, the antibody or antigen binding fragment thereof can bind C3b with a KD of less than or equal to 9 pM, less than or equal to 8 pM, less than or equal to 7 pM, less than or equal to 6 pM, less than or equal to 5 pM, less than or equal to 4 pM, less than or equal to 3 pM, less than or equal to 2 pM, or as high as less than or equal to 1 pM. The antibodies or antigen binding fragments described herein may bind to cynomolgus C3b with a KD of less than or equal to 250 pM, less than or equal to 240 pM, less than or equal to 230 pM, less than or equal to 220 pM, less than or equal to 210 pM, less than or equal to 200 pM, less than or equal to 190 pM, less than or equal to 180 pM, less than or equal to 170 pM, less than or equal to 160 pM, less than or equal to 150 pM, less than or equal to 140 pM, less than or equal to 130 pM, less than or equal to 120 pM, less than or equal to 110 pM, less than or equal to 100 pM, less than or equal to 90 pM, less than or equal to 80 pM, less than or equal to 70 pM, less than or equal to 60 pM, less than or equal to 50 pM, less than or equal to 40, 39, 38, 37, 36, 35, 34, 33, 32, or 31 pM, less than or equal to 30 pM, less than or equal to 20, 19, 18, 17, 16, 15, 14, 13, 12, or 11 pM and preferably as high as less than or equal to 10, 9, 8, 7, 6, 5, 4, 3, 2, or 1 pM.

Preferably the binding affinity of antibodies described herein is determined by solution equilibrium titration (SET). Methods for SET are known in the art and are described in further detail below.

The antibodies of the invention can be used to inhibit the alternative complement pathway. For example, an antibody or fragment thereof as described herein can inhibit the alternative complement pathway as measured by an in vitro hemolytic assay with an IC50 of less than or equal to 70 nM, preferably less than or equal to 65 nM, preferably less than or equal to 50 nM, preferably less than or equal to 40 nM, 30 nM, or 20 nM, and more preferably less than or equal to 10 nM. An antibody or fragment thereof as described herein can inhibit the alternative complement pathway in cynomolgus as measured by an in vitro hemolytic assay with an IC50 of less than or equal to 100 nM, preferably less than or equal to 90 nM, preferably less than or equal to 80 nM, preferably less than or equal to 75 nM, and more preferably less than or equal to 70 nM. An antibody or fragment thereof as described herein can inhibit the alternative complement pathway as measured by in vitro C3b deposition with an IC50 of less than or equal to 30 nM, less than or equal to 25 nM, less than or equal to 20 nM, and preferably less than or equal to 10 nM. An antibody or fragment thereof as described herein can inhibit the alternative complement pathway in cynomolgus as measured by in vitro C3b deposition with an IC50 of less than or equal to 70 nM, less than or equal to 50 nM, less than or equal to 40 nM, and preferably less than or equal to 30 nM.

An antibody or fragment thereof as described herein can inhibit the alternative complement pathway with an IC50 of less than or equal to 5 nM, preferably less than or equal to 4 nM, 3 nM, 2 nM, and more preferably less than or equal to 1 nM as measured by deposition of the complement membrane attack complex. An antibody or fragment thereof as described herein can inhibit the alternative complement pathway in cynomolgus with an IC50 of less than or equal to 20 nM, preferably less than or equal to 19 nM, 18 nM, 17 nM, 16 nM, 15 nM, 14 nM, or 13 nM, and more preferably less than or equal to 10 nM as measured by deposition of the complement membrane attack complex.

An antibody or fragment thereof as described herein can inhibit the alternative complement pathway with an IC50 of less than or equal to 100 nM, preferably less than or equal to 90 nM, 80 nM, 70 nM, 60 nM, 50 nM, 40 nM, 30 nM, or 20 nM, and more preferably less than or equal to 10 nM, as measured by generation of C3a and C5a

An antibody or antigen binding fragment thereof described in the invention preferably has the binding characteristics of a Fab as shown in Table 12.

The invention also includes an isolated antibody or antigen binding fragment thereof that specifically binds to human or cynomolgus complement C3b protein, and cross competes with an antibody described in Table 1.

The antibody or antigen binding fragment thereof as described herein can be a monoclonal antibody, a human or humanized antibody, a chimeric antibody, a single chain antibody, a Fab fragment, Fv fragment, F(ab′)2 fragment, or ScFv fragment, and/or an IgG isotype.

The antibodies of the invention can include a framework in which an amino acid has been substituted into the antibody framework from the respective human VH or VL germline sequences.

In one aspect, the antibodies of the invention bind to C3b with an affinity that is at least 1000 fold greater than the affinity of said antibody binding to C3.

The invention includes an antibody or antigen binding fragment thereof having the heavy and light chain sequences of antibody 9556 in Table 1. The invention includes an antibody or antigen binding fragment thereof having the heavy and light chain sequences of antibody 9611 in Table 1. The invention includes an antibody or antigen binding fragment thereof having the heavy and light chain sequences of antibody 9612 in Table 1. The invention also includes an antibody or antigen binding fragment thereof having the heavy and light chain sequences of antibody 9609 in Table 1. The invention includes an antibody or antigen binding fragment thereof having the heavy and light chain sequences of antibody 9610 in Table 1. The invention further includes an antibody or antigen binding fragment thereof having the heavy and light chain sequences of antibody 9674 in Table 1. The invention still further includes an antibody or antigen binding fragment thereof having the heavy and light chain sequences of antibody 9675 in Table 1. The invention includes an antibody or antigen binding fragment thereof having the heavy and light chain sequences of antibody 9124 in Table 1. The invention also includes an antibody or antigen binding fragment thereof having the heavy and light chain sequences of antibody 9397 in Table 1. The invention also includes an antibody or antigen binding fragment thereof having the heavy and light chain sequences of antibody 9398 in Table 1. The invention further includes an antibody or antigen binding fragment thereof having the heavy and light chain sequences of antibody 9136 in Table 1. The invention also includes an antibody or antigen binding fragment thereof having the heavy and light chain sequences of antibody 9141 in Table 1. The invention still further includes an antibody or antigen binding fragment thereof having the heavy and light chain sequences of antibody 9373 in Table 1. The invention also includes an antibody or antigen binding fragment thereof having the heavy and light chain sequences of antibody 9423 in Table 1.

The invention includes an antibody or antigen binding fragment thereof having the heavy and light chain variable domain sequences of antibody 9556 in Table 1. The invention includes an antibody or antigen binding fragment thereof having the heavy and light chain variable domain sequences of antibody 9611 in Table 1. The invention includes an antibody or antigen binding fragment thereof having the heavy and light chain variable domain sequences of antibody 9612 in Table 1. The invention also includes an antibody or antigen binding fragment thereof having the heavy and light chain variable domain sequences of antibody 9609 in Table 1. The invention includes an antibody or antigen binding fragment thereof having the heavy and light chain variable domain sequences of antibody 9610 in Table 1. The invention further includes an antibody or antigen binding fragment thereof having the heavy and light chain variable domain sequences of antibody 9674 in Table 1. The invention still further includes an antibody or antigen binding fragment thereof having the heavy and light chain variable domain sequences of antibody 9675 in Table 1. The invention includes an antibody or antigen binding fragment thereof having the heavy and light chain variable domain sequences of antibody 9124 in Table 1. The invention also includes an antibody or antigen binding fragment thereof having the heavy and light chain variable domain sequences of antibody 9397 in Table 1. The invention also includes an antibody or antigen binding fragment thereof having the heavy and light chain variable domain sequences of antibody 9398 in Table 1. The invention further includes an antibody or antigen binding fragment thereof having the heavy and light chain variable domain sequences of antibody 9136 in Table 1. The invention also includes an antibody or antigen binding fragment thereof having the heavy and light chain variable domain sequences of antibody 9141 in Table 1. The invention still further includes an antibody or antigen binding fragment thereof having the heavy and light chain variable domain sequences of antibody 9373 in Table 1. The invention also includes an antibody or antigen binding fragment thereof having the heavy and light chain variable domain sequences of antibody 9423 in Table 1.

The invention also relates to an isolated antibody or antigen binding fragment thereof that includes a heavy chain CDR1 selected from the group consisting of SEQ ID NOs 1, 15, 29, 43, 57, 71, 85, 99, 113, 127, 141, 155, 169, and 183; a heavy chain CDR2 selected from the group consisting of SEQ ID NOs: 2, 16, 30, 44, 58, 72, 86, 100, 114, 128, 142, 156, 170, and 184; and a heavy chain CDR3 selected from the group consisting of SEQ ID NOs: 3, 17, 31, 45, 59, 73, 87, 101, 115, 129, 143, 157, 171, and 185, wherein said isolated antibody or antigen binding fragment thereof binds to complement protein C3b. In a further aspect, the isolated antibody or antigen binding fragment thereof further includes a light chain CDR1 selected from the group consisting of SEQ ID NOs: 4, 18, 32, 46, 60, 74, 88, 102, 116, 130, 144, 158, 172, and 186; alight chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188.

The invention also relates to an isolated antibody or antigen binding fragment thereof that includes a light chain CDR1 selected from the group consisting of SEQ ID NOs: 4, 18, 32, 46, 60, 74, 88, 102, 116, 130, 144, 158, 172, and 186; a light chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188, wherein said isolated antibody or antigen binding fragment thereof binds to complement protein C3b.

The invention also relates to an isolated antibody or antigen binding fragment thereof that includes a heavy chain variable domain sequence selected from the group consisting of SEQ ID NOs: 7, 21, 35, 49, 63, 77, 91, 105, 119, 133, 147, 161, 175, and 189, and further includes a light chain variable domain sequence selected from the group consisting of SEQ ID NOs: 8, 22, 36, 50, 64, 78, 92, 106, 120, 134, 148, 162, 176, and 190, wherein said isolated antibody or antigen binding fragment thereof binds to complement protein C3b.

The invention also relates to an isolated antibody or antigen binding fragment thereof that includes a light chain variable domain sequence selected from the group consisting of SEQ ID NOs: 8, 22, 36, 50, 64, 78, 92, 106, 120, 134, 148, 162, 176, and 190, wherein said isolated antibody or antigen binding fragment thereof binds to complement protein C3b

The invention also relates to an isolated antibody or antigen binding fragment thereof, that includes a heavy chain variable domain having at least 95% sequence identity to a sequence selected from the group consisting of SEQ ID NOs: 7, 21, 35, 49, 63, 77, 91, 105, 119, 133, 147, 161, 175, and 189, wherein said antibody binds to C3b. In one aspect, the isolated antibody or antigen binding fragment thereof also includes a light chain variable domain having at least 95% sequence identity to a sequence selected from the group consisting of SEQ ID NOs 8, 22, 36, 50, 64, 78, 92, 106, 120, 134, 148, 162, 176, and 190.

The invention also relates to an isolated antibody or antigen binding fragment thereof, that includes a light chain variable domain having at least 95% sequence identity to a sequence selected from the group consisting of SEQ ID NOs 8, 22, 36, 50, 64, 78, 92, 106, 120, 134, 148, 162, 176, and 190, wherein said antibody binds C3b.

The invention still further relates to an isolated antibody or antigen binding fragment thereof that includes a heavy chain having at least 95% sequence identity to a sequence selected from the group consisting of SEQ ID NOs 9, 23, 37, 51, 65, 79, 93, 107, 121, 135, 149, 163, 177, and 191, wherein said antibody binds to C3b. In one aspect, the isolated antibody or antigen binding fragment thereof also includes a light chain having at least 95% sequence identity to a sequence selected from the group consisting of SEQ ID NOs 10, 24, 38, 52, 66, 80, 94, 108, 122, 136, 150, 164, 178, and 192.

The invention still further relates to an isolated antibody or antigen binding fragment thereof that includes a light chain having at least 95% sequence identity to a sequence selected from the group consisting of SEQ ID NOs 10, 24, 38, 52, 66, 80, 94, 108, 122, 136, 150, 164, 178, and 192, wherein said antibody binds C3b.

The invention also includes pharmaceutical compositions comprising the antibody compositions described herein as well as a pharmaceutically acceptable carrier. Specifically, the invention includes a pharmaceutical composition comprising an antibody or antigen binding fragment thereof of Table 1, such as, for example antibody 9556, 9611, 9612, 9609, 9610, 9674, 9675, 9124, 9397, 9398, 9136, 9141, 9373, or 9423. The invention also includes a pharmaceutical composition comprising a combination of two or more of the antibodies or antigen binding fragments thereof of Table 1.

The invention also includes an isolated nucleic acid comprising a sequence encoding a polypeptide that includes a heavy chain variable domain having at least 95% sequence identity to a sequence selected from the group consisting of SEQ ID NOs: 7, 21, 35, 49, 63, 77, 91, 105, 119, 133, 147, 161, 175, and 189.

The invention also relates to an isolated nucleic acid comprising a sequence encoding a polypeptide that includes a light chain variable domain having at least 95% sequence identity to a sequence selected from the group consisting of SEQ ID NOs 8, 22, 36, 50, 64, 78, 92, 106, 120, 134, 148, 162, 176, and 190.

In one aspect, the invention also includes a vector that includes one or more of the nucleic acid molecules described herein.

The invention also includes an isolated host cell that includes a recombinant DNA sequence encoding a heavy chain of the antibody described above, and a second recombinant DNA sequence encoding a light chain of the antibody described, wherein said DNA sequences are operably linked to a promoter and are capable of being expressed in the host cell. It is contemplated that the antibody can be a human monoclonal antibody. It is also contemplated that the host cell is a non-human mammalian cell.

The invention still further relates to a method of treating age related macular degeneration where the method includes the step of administering to a subject in need thereof an effective amount of a composition comprising the antibody or fragments thereof described herein. It is contemplated that the subject is a human.

The invention also provides a method of inhibiting the alternative complement pathway in a subject where the method includes the step of administering to a subject in need thereof, an effective amount of a composition comprising an antibody or antigen binding fragment as described herein. In one aspect, the subject is a human.

The invention also provides a method for inhibiting binding of C3b to factors B, P, or H that includes contacting C3b with an anti-C3b antibody or fragment thereof as described herein.

The invention also provides a method for inhibiting C3 convertase, C4 convertase, and C3b-C3b dimer formation that includes contacting C3b with an anti-C3b antibody or fragment thereof.

The invention also includes an isolated antibody or an antigen binding fragment thereof, comprising at least one complementarity determining region (CDR) sequence having at least 80%, 85%, 90%, and up to at least 95% sequence identity to a sequence selected from the group consisting of SEQ ID NOs: 1, 2, 3, 4, 5, 6, 15, 16, 17, 18, 19, 20, 29, 30, 31, 32, 33, 34, 43, 44, 45, 46, 47, 48, 57, 58, 59, 60, 61, 62, 71, 72, 73, 74, 75, 76, 85, 86, 87, 88, 89, and 90, wherein said antibody binds to the complement protein C3b.

The invention also includes an isolated antibody or antigen binding fragment thereof, comprising at least one heavy chain CDR sequence selected from the group consisting of SEQ ID NOs: 1, 2, 3, 15, 16, 17, 29, 30, 31, 43, 44, 45, 57, 58, 59, 71, 72, 73, 85, 86, and 87, wherein said antibody binds C3b.

The invention also includes an isolated antibody or antigen binding fragment thereof, comprising at least one light chain CDR sequence selected from the group consisting of SEQ ID NOs: 4, 5, 6, 18, 19, 20, 32, 33, 34, 46, 47, 48, 60, 61, 62, 74, 75, 76, 88, 89, and 90, wherein said antibody binds C3b.

The invention also includes an isolated antigen binding polypeptide comprising a heavy chain CDR1 selected from the group consisting of SEQ ID NOs: 1, 15, 29, 43, 57, 71, and 85; a heavy chain CDR2 selected from the group consisting of SEQ ID NOs: 2, 16, 30, 44, 58, 72, and 86; and a heavy chain CDR3 selected from the group consisting of SEQ ID NOs: 3, 17, 31, 45, 59, 73, and 87, wherein said antigen binding polypeptide binds to complement protein C3b.

The invention still further includes an antigen binding polypeptide comprising a light chain CDR1 selected from the group consisting of SEQ ID NOs: 4, 18, 32, 46, 60, 74, and 88; a light chain CDR2 selected from the group consisting of SEQ ID NOs 5, 19, 33, 47, 61, 75, and 89; and a light chain CDR3 selected from the group consisting of SEQ ID NOs 6, 20, 34, 48, 62, 76, and 90, wherein said antigen binding polypeptide binds to complement protein C3b.

In addition to the antigen binding polypeptide including the heavy chain CDR sequences noted above, the antigen binding polypeptide can also include a light chain CDR1 selected from the group consisting of SEQ ID NOs: 4, 18, 32; 46, 60, 74, and 88; a light chain CDR2 selected from the group consisting of SEQ ID NOs 5, 19, 33, 47, 61, 75, and 89; and a light chain CDR3 selected from the group consisting of SEQ ID NOs 6, 20, 34, 48, 62, 76, and 90.

The antigen binding polypeptides described herein preferably bind C3b with a KD of less than or equal to 100 pM, preferably less than or equal to 10 pM, preferably less than or equal to 2 pM. In addition, it is preferred that the antigen binding polypeptide bind to both human and cynomolgus C3b.

The invention also includes a method of modulating C3b comprising administering to a subject in need thereof an effective amount of an antibody, antigen binding fragment thereof, or antigen binding polypeptide described herein.

Any of the foregoing antibodies or antigen binding fragments thereof may be a monoclonal antibody or antigen binding fragment thereof.

### DEFINITIONS

Unless defined otherwise, all technical and scientific terms used herein have the same meaning as commonly understood by those of ordinary skill in the art to which this invention pertains.

The term “antibody” as used herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding portion” of an antibody, as used herein, refers to one or more fragments of an intact antibody that retain the ability to specifically bind to a given antigen (e.g., C3b). Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883): Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8(10):1057-1062; and U.S. Pat. No. 5,641,870).

As used herein, the term “Affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “Avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with only one antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

Specific binding between two entities means a binding with an equilibrium constant (K_{A}) of at least 1×10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹. The phrase “specifically (or selectively) binds” to an antibody (e.g., a C3b-binding antibody) refers to a binding reaction that is determinative of the presence of a cognate antigen (e.g., a human C3b or cynomolgus C3b) in a heterogeneous population of proteins and other biologics. In addition to the equilibrium constant (KA) noted above, an C3b-binding antibody of the invention typically also has a dissociation rate constant (Kd) of about 1×10⁻² s⁻¹, 1×10⁻³ s⁻¹, 1×10⁻⁴ s⁻¹, 1×10⁻⁴ s⁻¹, or lower, and binds to C3b with an affinity that is at least 10-fold, preferably 100-fold, or up to 1000-fold or more greater than its affinity for binding to a non-specific antigen (e.g., C3). The phrases “an antibody recognizing an antigen” and “an antibody specific for an antigen” are used interchangeably herein with the term “an antibody which binds specifically to an antigen”.

As used herein “neo-epitopes” or “neo-antigens” are used interchangeably and are antigenic portions of proteins that are present on C3b after proteolytic cleavage of C3. These neo-epitopes are not accessible on C3 which has not been cleaved.

The term “conditions or disorders associated with macular degeneration” refers to any of a number of conditions in which the retinal macula degenerates or becomes dysfunctional, e.g., as a consequence of decreased growth of cells of the macula, increased death or rearrangement of the cells of the macula (e.g., RPE cells), loss of normal biological function, or a combination of these events. Macular degeneration results in the loss of integrity of the histoarchitecture of the cells and/or extracellular matrix of the normal macula and/or the loss of function of the cells of the macula. Examples of macular degeneration-related disorder include AMD, North Carolina macular dystrophy, Sorsby's fundus dystrophy, Stargardt's disease, pattern dystrophy, Best disease, dominant drusen, and malattia leventinese (radial drusen). The term also encompasses extramacular changes that occur prior to, or following dysfunction and/or degeneration of the macula. Thus, the term “macular degeneration-related disorder” also broadly includes any condition which alters or damages the integrity or function of the macula (e.g., damage to the RPE or Bruch's membrane). For example, the term encompasses retinal detachment, chorioretinal degenerations, retinal degenerations, photoreceptor degenerations, RPE degenerations, mucopolysaccharidoses, rod-cone dystrophies, cone-rod dystrophies and cone degenerations.

The term “complement component”, “complement proteins” or “complement component proteins” refers to the molecules that are involved in activation of the complement system. The classical pathway components include, e.g., C1q, C1r, C1s, C4, C2, C3, C5, C6, C7, C8, C9, and C5b-9 complex (membrane attack complex: MAC). The alternative pathway components include, e.g., Factor B, Factor D, Properdin, H and I.

The terms “modulation” or “modulate” are used interchangeably herein to refer to both upregulation (i.e., activation or stimulation (e.g., by agonizing or potentiating) and downregulation (i.e., inhibition or suppression (e.g., by antagonizing, decreasing or inhibiting)) of an activity or a biological process (e.g., complement process). “Modulates” is intended to describe both the upregulation or downregulation of a process. A process which is upregulated by a certain stimulant may be inhibited by an antagonist to that stimulant. Conversely, a process that is downregulated by a certain modifying agent may be inhibited by an agonist to that modifying agent.

The terms “complement pathway associated molecules,” “complement pathway molecules,” and “complement pathway associated proteins” are used interchangeably and refer to the various molecules that play a role in complement activation and the downstream cellular activities mediated by, responsive to, or triggered by the activated complement system. They include initiators of complement pathways (i.e., molecules that directly or indirectly triggers the activation of complement system), molecules that are produced or play a role during complement activation (e.g., complement proteins/enzymes such as C3, C5, C5b-9, Factor B, Factor D, MASP-1, and MASP-2), complement receptors or inhibitors (e.g., clusterin, vitronectin, CR1, or CD59), and molecules regulated or triggered by the activated complement system (e.g., membrane attack complex-inhibitory factor, MACIF; see, e.g., Sugita et al., J Biochem, 106:589-92, 1989). Thus, in addition to complement proteins noted herein, complement pathway associated molecules also include, e.g., C3/C5 convertase regulators (RCA) such as complement receptor type 1 (also termed CR1 or CD35), complement receptor type 2 (also termed CR2 or CD21), membrane cofactor protein (MCP or CD46), and C4bBP; MAC regulators such as vitronectin, clusterin (also termed “SP40, 40”), CRP, CD59, and homologous restriction factor (HRF); immunoglobulin chains such as Ig kappa, Ig lambda, or Ig gamma); C1 inhibitor; and other proteins such as CR3, CR4 (CD11b/18), and DAF (CD 55).

The term “cellular activities regulated by the complement pathway” include cell damage resulting from the C5b-9 attack complex, vascular permeability changes, contraction and migration of smooth muscle cells, T cell proliferation, immune adherence, aggregation of dendritic cells, monocytes, granulocyte and platelet, phagocytosis, migration and activation of neutrophils (PMN) and macrophages.

Further, activation of the complement pathways results in the increase of proinflammatory response contributed by the by-products within the complement pathway. Disorders associated with activation of the complement pathway include nephritis, asthma, reperfusion injury, hemodialysis, rheumatoid arthritis, systemic lupus, psoriasis, multiple sclerosis, transplantation, Alzheimer's disease, aHUS, MPGN II, or any other complement-mediated disease. Disorders associated with macular degeneration include AMD, North Carolina macular dystrophy, Sorsby's fundus dystrophy, Stargardt's disease, pattern dystrophy, Best disease, dominant drusen, and malattia leventinese (radial drusen), extramacular changes that occur prior to, or following dysfunction and/or degeneration of the macula, retinal detachment, chorioretinal degenerations, retinal degenerations, photoreceptor degenerations, RPE degenerations, mucopolysaccharidoses, rod-cone dystrophies, cone-rod dystrophies and cone degenerations.

As used herein, the term “subject” includes any human or nonhuman animal.

The term “nonhuman animal” includes all nonhuman vertebrates, e.g., mammals and non-mammals, such as nonhuman primates, rodents, rabbits, sheep, dogs, cats, horses, cows, birds, amphibians, reptiles, etc.

The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “complement C3b protein” or “C3b” are used interchangeably, and refers to the C3b protein in different species. For example, human C3b has the sequence as set in SEQ ID NO: 197 (A chain) and 198 (B chain). Human C3b can be obtained from Complement Technology Inc. (Tyler, Tex.). Cynomolgus C3b can be produced as illustrated in the Example section below.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The terms “cross-block”, “cross-blocked” and “cross-blocking” are used interchangeably herein to mean the ability of an antibody or other binding agent to interfere with the binding of other antibodies or binding agents to C3 in a standard competitive binding assay.

The ability or extent to which an antibody or other binding agent is able to interfere with the binding of another antibody or binding molecule to C3, and therefore whether it can be said to cross-block according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-blocking uses an ELISA-based approach.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the term “high affinity” for an IgG antibody or fragment thereof (e.g., a Fab fragment) refers to an antibody having a KD of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, “high affinity” binding can vary for other antibody isotypes. For example, “high affinity” binding for an IgM isotype refers to an antibody having a KD of 10⁻⁷ M or less, or 10⁻⁸ M or less. In one aspect, the anti-C3b antibodies or antigen binding fragments thereof described herein have a KD of less than or equal to 1 nM, preferably less than or equal to 200 pM, more preferably less than or equal to 100 pM, and still more preferably less than or equal to 10 pM.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” refers to an antibody that is substantially free of other antibodies having different antigenic specificities (e.g., an isolated antibody that specifically binds C3b is substantially free of antibodies that specifically bind antigens other than C3b). An isolated antibody that specifically binds C3b may, however, have cross-reactivity to other antigens. Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e. Kd/Ka) and is expressed as a molar concentration (M). K_{D} values for antibodies can be determined using methods well established in the art. A method for determining the K_{D} of an antibody is by using surface plasmon resonance, or using a biosensor system such as a Biacore® system.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “treating” includes the administration of compositions or antibodies to prevent or delay the onset of the symptoms, complications, or biochemical indicia of a disease (e.g., AMD), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “C3b activity” means the activity of the alternative complement pathway downstream of the generation of C3b, including, but not limited to, for example, activity of the C3 convertase, activity of the C5 convertase. C3b activity can be determined using the assays described herein such as, but not limited to hemolytic assays, assays that measure the generation of C3a and C5a, C3b deposition assay, and a membrane attack complex (MAC) deposition assay. As used herein, “change in C3b activity” or “modulation of C3b activity” refers to a measurement of C3b activity by one or more of the assays described herein, wherein C3b activity is increased or decreased by at least 10% relative to a relevant control. For example, an antibody or antigen binding fragment of the invention can be said to modulate C3b activity when the activity of C3b is decreased or increased in the presence of the antibody or fragment by at least 10% relative to the activity of C3b in the absence of the antibody or fragment.

As used herein, the terms “cyno” or “cynomolgus” refer to the cynomolgus monkey (Macaca fascicularis).

### BRIEF DESCRIPTION OF THE DRAWINGS

FIG. 1 shows that the C3b antibodies bind to C3b with at least 1000 fold higher selectivity relative to C3.

FIG. 2 shows an example of the ability of the anti-C3b antibodies to inhibit hemolysis in either 10% human or cynomolgus serum.

FIG. 3 shows an example of the ability of the C3b antibodies to inhibit production of C3b as a breakdown product of C3.

FIG. 4 shows exemplary data demonstrating the ability of the C3b antibodies to inhibit the deposition of MAC.

FIG. 5 shows that the C3b antibodies block alternative pathway-driven complement activation by inhibiting generation of C3a and C5a.

FIG. 6A shows an SDS-PAGE gel showing the inhibition of tick-over convertase enzyme activity. FIG. 6B shows the quantitation of inhibition of C3b generation in the gel in 6A. FIG. 6C shows that anti-C3b antibodies inhibit pre-formed C3 convertase enzyme activity.

FIG. 7 shows that the anti-C3b antibodies inhibit in vitro C5 convertase enzyme activity.

FIG. 8A shows the inhibition of Factor B binding to C3b by C3b antibodies. FIG. 8B shows inhibition of factor P binding to C3b by C3b antibodies. FIG. 8C shows inhibition of factor H binding to C3b by C3b antibodies. FIG. 8D shows inhibition of C3b-C3b dimer formation by C3b antibodies.

FIG. 9 shows the results of an antibody cross reactivity assay against C3d.

FIG. 10 shows the results of an antibody cross reactivity assay against C5.

FIG. 11 shows the results from binding assays performed to determine whether the C3b antibodies bind to iC3b or C3c.

FIG. 12 shows the results from species cross reactivity studies. FIG. 12A shows rat cross reactivity. FIG. 12B shows rabbit cross reactivity. FIG. 12C shows pig cross reactivity. FIG. 12D shows mouse cross reactivity. FIG. 12E shows guinea pig cross reactivity. FIG. 12F shows dog cross reactivity.

### DETAILED DESCRIPTION

The present invention is based, in part, on the discovery of antibody molecules that specifically bind to both human and cynomolgus C3b. The invention relates to both full IgG format antibodies (see, e.g., antibodies 9556, 9611, 9612, 9609, 9610, 9674, and 9675) as well as antigen binding fragments thereof, such as Fab fragments (e.g., see antibodies 9124, 9397, 9398, 9136, 9141, 9373, and 9423).

Accordingly, the present invention provides antibodies that specifically bind to complement C3b protein (e.g., human C3b, cynomolgus C3b), pharmaceutical compositions, production methods, and methods of use of such antibodies and compositions.

### C3b Antibodies

The present invention provides antibodies that specifically bind to C3b (e.g., human C3b, cynomolgus C3b). In some embodiments, the present invention provides antibodies that specifically bind to both human and cynomolgus C3b. Antibodies of the invention include, but are not limited to, the human monoclonal antibodies, isolated as described, in the Examples.

The present invention provides antibodies that specifically bind a C3b protein (e.g., human and/or cynomolgus C3b, said antibodies comprising a VH domain haying an amino acid sequence of SEQ ID NO: 7, 21, 35, 49, 63, 77, 91, 105, 119, 133, 147, 161, 175, and 189. The present invention also provides antibodies that specifically bind to a C3b protein (e.g., human and/or cynomolgus C3b), said antibodies comprising a VH CDR having an amino acid sequence of any one of the VH CDRs listed in Table 1, infra. In particular, the invention provides antibodies that specifically bind to a C3b protein (e.g., human and/or cynomolgus C3b), said antibodies comprising (or alternatively, consisting of) one, two, three, four, five or more VH CDRs having an amino acid sequence of any of the VH CDRs listed in Table 1, infra.

The present invention provides antibodies that specifically bind to a C3b protein (e.g., human and/or cynomolgus C3b), said antibodies comprising a VL domain having an amino acid sequence of SEQ ID NO: 8, 22, 36, 50, 64, 78, 92, 106, 120, 134, 148, 162, 176, and 190. The present invention also provides antibodies that specifically bind to a C3b protein (e.g., human and/or cynomolgus C3b), said antibodies comprising a VL CDR having an amino acid sequence of any one of the VL CDRs listed in Table 1, infra. In particular, the invention provides antibodies that specifically bind to a C3b protein (e.g., human and/or cynomolgus C3b), said antibodies comprising (or alternatively, consisting of) one, two, three or more VL CDRs having an amino acid sequence of any of the VL CDRs listed in Table 1, infra.

Other antibodies of the invention include amino acids that have been mutated, yet have at least 60, 70, 80, 85, 90 or 95 percent identity in the CDR regions with the CDR regions depicted in the sequences described in Table 1. In some embodiments, it includes mutant amino acid sequences wherein no more than 1, 2, 3, 4 or 5 amino acids have been mutated in the CDR regions when compared with the CDR regions depicted in the sequence described in Table 1.

The present invention also provides nucleic acid sequences that encode VH, VL, the full length heavy chain, and the full length light chain of the antibodies that specifically bind to a C3b protein (e.g., human and/or cynomolgus C3b). Such nucleic acid sequences can be optimized for expression in mammalian cells (for example, Table 1 shows the optimized nucleic acid sequences for the heavy chain and light chain of antibodies 9556, 9611, 9612, 9609, 9610, 9674, and 9675, as well as Fab fragments 9124, 9397, 9398, 9136, 9141, 9373, and 9423).

TABLE 1
Table 1. Examples of C3b Antibodies, Fabs of the Present
Invention and C3b Proteins

| | |
|---|---|
| Amino acid | |
| sequence or | |
| polynucleotide | |
| (PN) | Sequence Identifier (SEQ.I.D.NO:)and sequence |

MOR09556

| | |
|---|---|
| CDRH1 | 1 |
| | SYWMT |
| CDRH2 | 2 |
| | SIKIKPDGYAASVKG |
| CDRH3 | 3 |
| | LFYQYFARMDY |
| CDRL1 | 4 |
| | RASQDISNYLN |
| CDRL2 | 5 |
| | AASNLQS |
| CDRL3 | 6 |
| | QQYDSYSPT |
| VH | 7 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSS |
| VL | 8 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQYDSYSPTFGQGTKVEI |
| | K |
| Heavy chain | 9 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSSASTKGPSVFPLAPSSKSTSGGTAALGCLVKDYFPEPVTVSW |
| | NSGALTSGVHTFPAVLQSSGLYSLSSVVTVPSSSLGTQTYICNVNHKPSNTKV |
| | DKRVEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQDWLNGK |
| | EYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVF |
| | SCSVMHEALHNHYTQKSLSLSPGK |
| Light chain | 10 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQYDSYSPTFGQGTKVEI |
| | KRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| PN encoding | 11 |
| SEQ.I.D.NO: 7 | GAGGTGCAATTGGTGGAATCTGGCGGCGGACTGGTGCAGCCTGGCGGCAGCCT |
| | GAGACTGAGCTGCGCCGCCAGCGGCTTCACCTTCAGCAGCTACTGGATGACAT |
| | GGGTGCGCCAGGCCCCTGGCAAGGGACTGGAATGGGTGTCCAGCATCAAGATC |
| | AAGCCCGACGGCTACGCCGCCTCCGTGAAGGGCCGGTTCACCATCAGCCGGGA |
| | CAACAGCAAGAACACCCTGTACCTGCAGATGAACAGCCTGCGGGCCGAGGACA |
| | CCGCCGTGTACTACTGCGCCAGACTGTTCTACCAGTACTTCGCCCGGATGGAC |
| | TACTGGGGCCAGGGCACCCTGGTGACCGTGAGCTCA |
| PN encoding | 12 |
| SEQ.I.D.NO: 8 | GATATCCAGATGACCCAGAGCCCCAGCAGCCTGAGCGCCAGCGTGGGCGACAG |
| | AGTGACCATCACCTGCCGGGCCAGCCAGGACATCAGCAACTACCTGAACTGGT |
| | ATCAGCAGAAGCCCGGCAAGGCCCCCAAGCTGCTGATCTACGCCGCCAGCAAC |
| | CTGCAGAGCGGCGTGCCCAGCCGGTTTAGCGGCAGCGGCTCCGGCACCGACTT |
| | CACCCTGACCATCAGCTCCCTGCAGCCCGAGGACTTCGCCACCTACTACTGCC |
| | AGCAGTACGACAGCTACAGCCCCACCTTCGGCCAGGGCACCAAGGTGGAGATC |
| | AAG |
| PN encoding | 13 |
| SEQ.I.D.NO: 9 | ATGAAGCACCTGTGGTTCTTTCTGCTGCTGGTCGCCGCTCCCAGATGGGTGCT |
| | GTCCGAGGTGCAATTGGTGGAATCTGGCGGCGGACTGGTGCAGCCTGGCGGCA |
| | GCCTGAGACTGAGCTGCGCCGCCAGCGGCTTCACCTTCAGCAGCTACTGGATG |
| | ACATGGGTGCGCCAGGCCCCTGGCAAGGGACTGGAATGGGTGTCCAGCATCAA |
| | GATCAAGCCCGACGGCTACGCCGCCTCCGTGAAGGGCCGGTTCACCATCAGCC |
| | GGGACAACAGCAAGAACACCCTGTACCTGCAGATGAACAGCCTGCGGGCCGAG |
| | GACACCGCCGTGTACTACTGCGCCAGACTGTTCTACCAGTACTTCGCCCGGAT |
| | GGACTACTGGGGCCAGGGCACCCTGGTGACCGTGAGCTCAGCCTCCACCAAGG |
| | GTCCATCGGTCTTCCCCCTGGCACCCTCCTCCAAGAGCACCTCTGGGGGCACA |
| | GCGGCCCTGGGCTGCCTGGTCAAGGACTACTTCCCCGAACCGGTGACGGTGTC |
| | GTGGAACTCAGGCGCCCTGACCAGCGGCGTGCACACCTTCCCGGCTGTCCTAC |
| | AGTCCTCAGGACTCTACTCCCTCAGCAGCGTGGTGACCGTGCCCTCCAGCAGC |
| | TTGGGCACCCAGACCTACATCTGCAACGTGAATCACAAGCCCAGCAACACCAA |
| | GGTGGACAAGAGAGTTGAGCCCAAATCTTGTGACAAAACTCACACATGCCCAC |
| | CGTGCCCAGCACCTGAAGCAGCGGGGGGACCGTCAGTCTTCCTCTTCCCCCCA |
| | AAACCCAAGGACACCCTCATGATCTCCCGGACCCCTGAGGTCACATGCGTGGT |
| | GGTGGACGTGAGCCACGAAGACCCTGAGGTCAAGTTCAACTGGTACGTGGACG |
| | GCGTGGAGGTGCATAATGCCAAGACAAAGCCGCGGGAGGAGCAGTACAACAGC |
| | ACGTACCGGGTGGTCAGCGTCCTCACCGTCCTGCACCAGGACTGGCTGAATGG |
| | CAAGGAGTACAAGTGCAAGGTCTCCAACAAAGCCCTCCCAGCCCCCATCGAGA |
| | AAACCATCTCCAAAGCCAAAGGGCAGCCCCGAGAACCACAGGTGTACACCCTG |
| | CCCCCATCCCGGGAGGAGATGACCAAGAACCAGGTCAGCCTGACCTGCCTGGT |
| | CAAAGGCTTCTATCCCAGCGACATCGCCGTGGAGTGGGAGAGCAATGGGCAGC |
| | CGGAGAACAACTACAAGACCACGCCTCCCGTGCTGGACTCCGACGGCTCCTTC |
| | TTCCTCTACAGCAAGCTCACCGTGGACAAGAGCAGGTGGCAGCAGGGGAACGT |
| | CTTCTCATGCTCCGTGATGCATGAGGCTCTGCACAACCACTACACGCAGAAGA |
| | GCCTCTCCCTGTCTCCGGGTAAATGA |
| PN encoding | 14 |
| SEQ.I.D.NO: 10 | GATATCCAGATGACCCAGAGCCCCAGCAGCCTGAGCGCCAGCGTGGGCGACAG |
| | AGTGACCATCACCTGCCGGGCCAGCCAGGACATCAGCAACTACCTGAACTGGT |
| | ATCAGCAGAAGCCCGGCAAGGCCCCCAAGCTGCTGATCTACGCCGCCAGCAAC |
| | CTGCAGAGCGGCGTGCCCAGCCGGTTTAGCGGCAGCGGCTCCGGCACCGACTT |
| | CACCCTGACCATCAGCTCCCTGCAGCCCGAGGACTTCGCCACCTACTACTGCC |
| | AGCAGTACGACAGCTACAGCCCCACCTTCGGCCAGGGCACCAAGGTGGAGATC |
| | AAGCGTACGGTGGCTGCACCATCTGTCTTCATCTTCCCGCCATCTGATGAGCA |
| | GTTGAAATCTGGAACTGCCTCTGTTGTGTGCCTGCTGAATAACTTCTATCCCA |
| | GAGAGGCCAAAGTACAGTGGAAGGTGGATAACGCCCTCCAATCGGGTAACTCC |
| | CAGGAGAGTGTCACAGAGCAGGACAGCAAGGACAGCACCTACAGCCTCAGCAG |
| | CACCCTGACGCTGAGCAAAGCAGACTACGAGAAACACAAAGTCTACGCCTGCG |
| | AAGTCACCCATCAGGGCCTGAGCTCGCCCGTCACAAAGAGCTTCAACAGGGGA |
| | GAGTGT |

MOR09611

| | |
|---|---|
| CDRH1 | 15 |
| | SYWMT |
| CDRH2 | 16 |
| | SIKIKPDGYAASVKG |
| CDRH3 | 17 |
| | LFYQYFARMDY |
| CDRL1 | 18 |
| | RASQDISNYLN |
| CDRL2 | 19 |
| | AASNLQS |
| CDRL3 | 20 |
| | QQHDTFRPT |
| VH | 21 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSS |
| VL | 22 |
| | diqmtqspsslsasvgdrvtitcrasqdisnylnwyqqkpgkapklliyaasn |
| | lqsgvpsrfsgsgsgtdftltisslqpedfatyycqqhdtfrptfgqgtkvei |
| | k |
| Heavy chain | 23 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSSASTKGPSVFPLAPSSKSTSGGTAALGCLVKDYFPEPVTVSW |
| | NSGALTSGVHTFPAVLQSSGLYSLSSVVTVPSSSLGTQTYICNVNHKPSNTKV |
| | DKRVEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQDWLNGK |
| | EYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVF |
| | SCSVMHEALHNHYTQKSLSLSPGK |
| Light chain | 24 |
| | diqmtqspsslsasvgdrvtitcrasqdisnylnwyqqkpgkapklliyaasn |
| | lqsgvpsrfsgsgsgtdftltisslqpedfatyycqqhdtfrptfgqgtkvei |
| | krtvaapsvfifppsdeqlksgtasvvcllnnfypreakvqwkvdnalqsgns |
| | qesvteqdskdstyslsstltlskadyekhkvyacevthqglsspvtksfnrg |
| | ec |
| PN encoding | 25 |
| SEQ.I.D.NO: 21 | GAGGTGCAATTGGTGGAATCTGGCGGCGGACTGGTGCAGCCTGGCGGCAGCCT |
| | GAGACTGAGCTGCGCCGCCAGCGGCTTCACCTTCAGCAGCTACTGGATGACAT |
| | GGGTGCGCCAGGCCCCTGGCAAGGGACTGGAATGGGTGTCCAGCATCAAGATC |
| | AAGCCCGACGGCTACGCCGCCTCCGTGAAGGGCCGGTTCACCATCAGCCGGGA |
| | CAACAGCAAGAACACCCTGTACCTGCAGATGAACAGCCTGCGGGCCGAGGACA |
| | CCGCCGTGTACTACTGCGCCAGACTGTTCTACCAGTACTTCGCCCGGATGGAC |
| | TACTGGGGCCAGGGCACCCTGGTGACCGTGAGCTCA |
| PN encoding | 26 |
| SEQ.I.D.NO: 22 | gatatccagatgacccagagccccagcagcctgagcgccagcgtgggcgacag |
| | agtgaccatcacctgtcgggccagccaggacatcagcaactacctgaactggt |
| | atcagcagaagcccggcaaggcccccaagctgctgatctacgccgccagcaat |
| | ctgcagagcggcgtgcccagccggtttagcggcagcggctccggcaccgactt |
| | taccctgacaatttcctctctgcagcctgaggacttcgccacctactactgcc |
| | agcagcacgacaccttccggcccaccttcggccagggcaccaaggtggagatc |
| | aag |
| PN encoding | 27 |
| SEQ.I.D.NO: 23 | ATGAAGCACCTGTGGTTCTTTCTGCTGCTGGTCGCCGCTCCCAGATGGGTGCT |
| | GTCCGAGGTGCAATTGGTGGAATCTGGCGGCGGACTGGTGCAGCCTGGCGGCA |
| | GCCTGAGACTGAGCTGCGCCGCCAGCGGCTTCACCTTCAGCAGCTACTGGATG |
| | ACATGGGTGCGCCAGGCCCCTGGCAAGGGACTGGAATGGGTGTCCAGCATCAA |
| | GATCAAGCCCGACGGCTACGCCGCCTCCGTGAAGGGCCGGTTCACCATCAGCC |
| | GGGACAACAGCAAGAACACCCTGTACCTGCAGATGAACAGCCTGCGGGCCGAG |
| | GACACCGCCGTGTACTACTGCGCCAGACTGTTCTACCAGTACTTCGCCCGGAT |
| | GGACTACTGGGGCCAGGGCACCCTGGTGACCGTGAGCTCAGCCTCCACCAAGG |
| | GTCCATCGGTCTTCCCCCTGGCACCCTCCTCCAAGAGCACCTCTGGGGGCACA |
| | GCGGCCCTGGGCTGCCTGGTCAAGGACTACTTCCCCGAACCGGTGACGGTGTC |
| | GTGGAACTCAGGCGCCCTGACCAGCGGCGTGCACACCTTCCCGGCTGTCCTAC |
| | AGTCCTCAGGACTCTACTCCCTCAGCAGCGTGGTGACCGTGCCCTCCAGCAGC |
| | TTGGGCACCCAGACCTACATCTGCAACGTGAATCACAAGCCCAGCAACACCAA |
| | GGTGGACAAGAGAGTTGAGCCCAAATCTTGTGACAAAACTCACACATGCCCAC |
| | CGTGCCCAGCACCTGAAGCAGCGGGGGGACCGTCAGTCTTCCTCTTCCCCCCA |
| | AAACCCAAGGACACCCTCATGATCTCCCGGACCCCTGAGGTCACATGCGTGGT |
| | GGTGGACGTGAGCCACGAAGACCCTGAGGTCAAGTTCAACTGGTACGTGGACG |
| | GCGTGGAGGTGCATAATGCCAAGACAAAGCCGCGGGAGGAGCAGTACAACAGC |
| | ACGTACCGGGTGGTCAGCGTCCTCACCGTCCTGCACCAGGACTGGCTGAATGG |
| | CAAGGAGTACAAGTGCAAGGTCTCCAACAAAGCCCTCCCAGCCCCCATCGAGA |
| | AAACCATCTCCAAAGCCAAAGGGCAGCCCCGAGAACCACAGGTGTACACCCTG |
| | CCCCCATCCCGGGAGGAGATGACCAAGAACCAGGTCAGCCTGACCTGCCTGGT |
| | CAAAGGCTTCTATCCCAGCGACATCGCCGTGGAGTGGGAGAGCAATGGGCAGC |
| | CGGAGAACAACTACAAGACCACGCCTCCCGTGCTGGACTCCGACGGCTCCTTC |
| | TTCCTCTACAGCAAGCTCACCGTGGACAAGAGCAGGTGGCAGCAGGGGAACGT |
| | CTTCTCATGCTCCGTGATGCATGAGGCTCTGCACAACCACTACACGCAGAAGA |
| | GCCTCTCCCTGTCTCCGGGTAAATGA |
| PN encoding | 28 |
| SEQ.I.D.NO: 24 | gatatccagatgacccagagccccagcagcctgagcgccagcgtgggcgacag |
| | agtgaccatcacctgtcgggccagccaggacatcagcaactacctgaactggt |
| | atcagcagaagcccggcaaggcccccaagctgctgatctacgccgccagcaat |
| | ctgcagagcggcgtgcccagccggtttagcggcagcggctccggcaccgactt |
| | taccctgacaatttcctctctgcagcctgaggacttcgccacctactactgcc |
| | agcagcacgacaccttccggcccaccttcggccagggcaccaaggtggagatc |
| | aagcgtacggtggctgcaccatctgtcttcatcttcccgccatctgatgagca |
| | gttgaaatctggaactgcctctgttgtgtgcctgctgaataacttctatccca |
| | gagaggccaaagtacagtggaaggtggataacgccctccaatcgggtaactcc |
| | caggagagtgtcacagagcaggacagcaaggacagcacctacagcctcagcag |
| | caccctgacgctgagcaaagcagactacgagaaacacaaagtctacgcctgcg |
| | aagtcacccatcagggcctgagctcgcccgtcacaaagagcttcaacagggga |
| | gagtgt |

MOR09612

| | |
|---|---|
| CDRH1 | 29 |
| | SYWMT |
| CDRH2 | 30 |
| | SIKIKPDGYAASVKG |
| CDRH3 | 31 |
| | LFYQYFARMDY |
| CDRL1 | 32 |
| | RASQDISNYLN |
| CDRL2 | 33 |
| | AASNLQS |
| CDRL3 | 34 |
| | QQWDSFSPT |
| VH | 35 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSS |
| VL | 36 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQWDSFSPTFGQGTKVEI |
| | K |
| Heavy chain | 37 |
| | EVQLVESGGGLVQPGGSLRLSCAASGFTFSSYWMTWVRQAPGKGLEWVSSIKI |
| | KPDGYAASVKGRFTISRDNSKNTLYLQMNSLRAEDTAVYYCARLFYQYFARMD |
| | YWGQGTLVTVSSASTKGPSVFPLAPSSKSTSGGTAALGCLVKDYFPEPVTVSW |
| | NSGALTSGVHTFPAVLQSSGLYSLSSVVTVPSSSLGTQTYICNVNHKPSNTKV |
| | DKRVEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQDWLNGK |
| | EYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVF |
| | SCSVMHEALHNHYTQKSLSLSPGK |
| Light chain | 38 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQWDSFSPTFGQGTKVEI |
| | KRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| PN encoding | 39 |
| SEQ.I.D.NO: 35 | GAGGTGCAATTGGTGGAATCTGGCGGCGGACTGGTGCAGCCTGGCGGCAGCCT |
| | GAGACTGAGCTGCGCCGCCAGCGGCTTCACCTTCAGCAGCTACTGGATGACAT |
| | GGGTGCGCCAGGCCCCTGGCAAGGGACTGGAATGGGTGTCCAGCATCAAGATC |
| | AAGCCCGACGGCTACGCCGCCTCCGTGAAGGGCCGGTTCACCATCAGCCGGGA |
| | CAACAGCAAGAACACCCTGTACCTGCAGATGAACAGCCTGCGGGCCGAGGACA |
| | CCGCCGTGTACTACTGCGCCAGACTGTTCTACCAGTACTTCGCCCGGATGGAC |
| | TACTGGGGCCAGGGCACCCTGGTGACCGTGAGCTCA |
| PN encoding | 40 |
| SEQ.I.D.NO: 36 | GATATCCAGATGACCCAGAGCCCCAGCAGCCTGAGCGCCAGCGTGGGCGACAG |
| | AGTGACCATCACCTGTCGGGCCAGCCAGGACATCAGCAACTACCTGAACTGGT |
| | ATCAGCAGAAGCCCGGCAAGGCCCCCAAGCTGCTGATCTACGCCGCCAGCAAT |
| | CTGCAGAGCGGCGTGCCCAGCCGGTTTAGCGGCAGCGGCTCCGGCACCGACTT |
| | TACCCTGACAATCTCCTCTCTGCAGCCTGAGGACTTCGCCACCTACTACTGCC |
| | AGCAGTGGGACAGCTTCAGCCCCACCTTCGGCCAGGGCACCAAGGTGGAGATC |
| | AAG |
| PN encoding | 41 |
| SEQ.I.D.NO: 37 | ATGAAGCACCTGTGGTTCTTTCTGCTGCTGGTCGCCGCTCCCAGATGGGTGCT |
| | GTCCGAGGTGCAATTGGTGGAATCTGGCGGCGGACTGGTGCAGCCTGGCGGCA |
| | GCCTGAGACTGAGCTGCGCCGCCAGCGGCTTCACCTTCAGCAGCTACTGGATG |
| | ACATGGGTGCGCCAGGCCCCTGGCAAGGGACTGGAATGGGTGTCCAGCATCAA |
| | GATCAAGCCCGACGGCTACGCCGCCTCCGTGAAGGGCCGGTTCACCATCAGCC |
| | GGGACAACAGCAAGAACACCCTGTACCTGCAGATGAACAGCCTGCGGGCCGAG |
| | GACACCGCCGTGTACTACTGCGCCAGACTGTTCTACCAGTACTTCGCCCGGAT |
| | GGACTACTGGGGCCAGGGCACCCTGGTGACCGTGAGCTCAGCCTCCACCAAGG |
| | GTCCATCGGTCTTCCCCCTGGCACCCTCCTCCAAGAGCACCTCTGGGGGCACA |
| | GCGGCCCTGGGCTGCCTGGTCAAGGACTACTTCCCCGAACCGGTGACGGTGTC |
| | GTGGAACTCAGGCGCCCTGACCAGCGGCGTGCACACCTTCCCGGCTGTCCTAC |
| | AGTCCTCAGGACTCTACTCCCTCAGCAGCGTGGTGACCGTGCCCTCCAGCAGC |
| | TTGGGCACCCAGACCTACATCTGCAACGTGAATCACAAGCCCAGCAACACCAA |
| | GGTGGACAAGAGAGTTGAGCCCAAATCTTGTGACAAAACTCACACATGCCCAC |
| | CGTGCCCAGCACCTGAAGCAGCGGGGGGACCGTCAGTCTTCCTCTTCCCCCCA |
| | AAACCCAAGGACACCCTCATGATCTCCCGGACCCCTGAGGTCACATGCGTGGT |
| | GGTGGACGTGAGCCACGAAGACCCTGAGGTCAAGTTCAACTGGTACGTGGACG |
| | GCGTGGAGGTGCATAATGCCAAGACAAAGCCGCGGGAGGAGCAGTACAACAGC |
| | ACGTACCGGGTGGTCAGCGTCCTCACCGTCCTGCACCAGGACTGGCTGAATGG |
| | CAAGGAGTACAAGTGCAAGGTCTCCAACAAAGCCCTCCCAGCCCCCATCGAGA |
| | AAACCATCTCCAAAGCCAAAGGGCAGCCCCGAGAACCACAGGTGTACACCCTG |
| | CCCCCATCCCGGGAGGAGATGACCAAGAACCAGGTCAGCCTGACCTGCCTGGT |
| | CAAAGGCTTCTATCCCAGCGACATCGCCGTGGAGTGGGAGAGCAATGGGCAGC |
| | CGGAGAACAACTACAAGACCACGCCTCCCGTGCTGGACTCCGACGGCTCCTTC |
| | TTCCTCTACAGCAAGCTCACCGTGGACAAGAGCAGGTGGCAGCAGGGGAACGT |
| | CTTCTCATGCTCCGTGATGCATGAGGCTCTGCACAACCACTACACGCAGAAGA |
| | GCCTCTCCCTGTCTCCGGGTAAATGA |
| PN encoding | 42 |
| SEQ.I.D.NO: 38 | GATATCCAGATGACCCAGAGCCCCAGCAGCCTGAGCGCCAGCGTGGGCGACAG |
| | AGTGACCATCACCTGTCGGGCCAGCCAGGACATCAGCAACTACCTGAACTGGT |
| | ATCAGCAGAAGCCCGGCAAGGCCCCCAAGCTGCTGATCTACGCCGCCAGCAAT |
| | CTGCAGAGCGGCGTGCCCAGCCGGTTTAGCGGCAGCGGCTCCGGCACCGACTT |
| | TACCCTGACAATCTCCTCTCTGCAGCCTGAGGACTTCGCCACCTACTACTGCC |
| | AGCAGTGGGACAGCTTCAGCCCCACCTTCGGCCAGGGCACCAAGGTGGAGATC |
| | AAGCGTACGGTGGCTGCACCATCTGTCTTCATCTTCCCGCCATCTGATGAGCA |
| | GTTGAAATCTGGAACTGCCTCTGTTGTGTGCCTGCTGAATAACTTCTATCCCA |
| | GAGAGGCCAAAGTACAGTGGAAGGTGGATAACGCCCTCCAATCGGGTAACTCC |
| | CAGGAGAGTGTCACAGAGCAGGACAGCAAGGACAGCACCTACAGCCTCAGCAG |
| | CACCCTGACGCTGAGCAAAGCAGACTACGAGAAACACAAAGTCTACGCCTGCG |
| | AAGTCACCCATCAGGGCCTGAGCTCGCCCGTCACAAAGAGCTTCAACAGGGGA |
| | GAGTGT |

MOR09609

| | |
|---|---|
| CDRH1 | 43 |
| | SYTFS |
| CDRH2 | 44 |
| | NILPIFGDANYAQKFQG |
| CDRH3 | 45 |
| | nkgafyymstypsldv |
| CDRL1 | 46 |
| | RASQNINYYLN |
| CDRL2 | 47 |
| | DAFSLQS |
| CDRL3 | 48 |
| | QQSWSVPPFT |
| VH | 49 |
| | evqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvss |
| VL | 50 |
| | diqmtqspsslsasvgdrvtitcrasqninyylnwyqqkpgkapklliydafs |
| | lqsgvpsrfsgsgsgtdftltisslqpedfatyycqqswsvppftfgqgtkve |
| | ik |
| Heavy chain | 51 |
| | evqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvssastkgpsvfplapsskstsggtaalgclvkdyfp |
| | epvtvswnsgaltsgvhtfpavlqssglyslssvvtvpssslgtqtyicnvnh |
| | kpsntkvdkrvepkscdkthtcppcpapeaaggpsvflfppkpkdtlmisrtp |
| | evtcvvvdvshedpevkfnwyvdgvevhnaktkpreeqynstyrvvsvltvlh |
| | qdwlngkeykckvsnkalpapiektiskakgqprepqvytlppsreemtknqv |
| | sltclvkgfypsdiavewesngqpennykttppvldsdgsfflyskltvdksr |
| | wqqgnvfscsvmhealhnhytqkslslspgk |
| Light chain | 52 |
| | diqmtqspsslsasvgdrvtitcrasqninyylnwyqqkpgkapklliydafs |
| | lqsgvpsrfsgsgsgtdftltisslqpedfatyycqqswsvppftfgqgtkve |
| | ikrtvaapsvfifppsdeqlksgtasvvcllnnfypreakvqwkvdnalqsgn |
| | sqesvteqdskdstyslsstltlskadyekhkvyacevthqglsspvtksfnr |
| | gec |
| PN encoding | 53 |
| SEQ.I.D.NO: 49 | gaggtgcaattggtgcagagcggagccgaagtgaagaagcccggcagcagcgt |
| | caaggtgtcctgcaaggccagcggcggcaccttcagcagctacaccttcagct |
| | gggtgcgccaggccccaggacagggcctggaatggatgggcaacatcctgccc |
| | atcttcggcgacgccaactacgcccagaagttccagggcagagtcaccatcac |
| | cgccgacgagagcaccagcaccgcctacatggaactgagcagcctgcggagcg |
| | aggacaccgccgtgtactactgcgcccggaacaagggcgccttctactacatg |
| | agcacctaccccagcctggacgtgtggggccagggcaccctggtgaccgtgag |
| | ctca |
| PN encoding | 54 |
| SEQ.I.D.NO: 50 | gatatccagatgacccagagccccagcagcctgagcgccagcgtgggcgacag |
| | agtgaccatcacctgtcgggccagccagaacatcaactactacctgaactggt |
| | atcagcagaagcccggcaaggcccccaagctgctgatctacgacgccttcagc |
| | ctgcagagcggcgtgcccagccggtttagcggcagcggctccggcaccgactt |
| | taccctgacaatttcctctctgcagcctgaggacttcgccacctactactgcc |
| | agcagtcttggagcgtgccccccttcaccttcggccagggcaccaaggtggag |
| | atcaag |
| PN encoding | 55 |
| SEQ.I.D.NO: 51 | gaggtgcaattggtgcagagcggagccgaagtgaagaagcccggcagcagcgt |
| | caaggtgtcctgcaaggccagcggcggcaccttcagcagctacaccttcagct |
| | gggtgcgccaggccccaggacagggcctggaatggatgggcaacatcctgccc |
| | atcttcggcgacgccaactacgcccagaagttccagggcagagtcaccatcac |
| | cgccgacgagagcaccagcaccgcctacatggaactgagcagcctgcggagcg |
| | aggacaccgccgtgtactactgcgcccggaacaagggcgccttctactacatg |
| | agcacctaccccagcctggacgtgtggggccagggcaccctggtgaccgtgag |
| | ctcagcctccaccaagggtccatcggtcttccccctggcaccctcctccaaga |
| | gcacctctgggggcacagcggccctgggctgcctggtcaaggactacttcccc |
| | gaaccggtgacggtgtcgtggaactcaggcgccctgaccagcggcgtgcacac |
| | cttcccggctgtcctacagtcctcaggactctactccctcagcagcgtggtga |
| | ccgtgccctccagcagcttgggcacccagacctacatctgcaacgtgaatcac |
| | aagcccagcaacaccaaggtggacaagagagttgagccccaaatcttgtgacaa |
| | aactcacacatgcccaccgtgcccagcacctgaagcagcggggggaccgtcag |
| | tcttcctcttccccccaaaacccaaggacaccctcatgatctcccggacccct |
| | gaggtcacatgcgtggtggtggacgtgagccacgaagaccctgaggtcaagtt |
| | caactggtacgtggacggcgtggaggtgcataatgccaagacaaagccgcggg |
| | aggagcagtacaacagcacgtaccgggtggtcagcgtcctcaccgtcctgcac |
| | caggactggctgaatggcaaggagtacaagtgcaaggtctccaacaaagccct |
| | cccagcccccatcgagaaaaccatctccaaagccaaagggcagccccgagaac |
| | cacaggtgtacaccctgcccccatcccgggaggagatgaccaagaaccaggtc |
| | agcctgacctgcctggtcaaaggcttctatcccagcgacatcgccgtggagtg |
| | ggagagcaatgggcagccggagaacaactacaagaccacgcctcccgtgctgg |
| | actccgacggctccttcttcctctacagcaagctcaccgtggacaagagcagg |
| | tggcagcaggggaacgtcttctcatgctccgtgatgcatgaggctctgcacaa |
| | ccactacacgcagaagagcctctccctgtctccgggtaaa |
| PN encoding | 56 |
| SEQ.I.D.NO: 52 | gatatccagatgacccagagccccagcagcctgagcgccagcgtgggcgacag |
| | agtgaccatcacctgtcgggccagccagaacatcaactactacctgaactggt |
| | atcagcagaagcccggcaaggcccccaagctgctgatctacgacgccttcagc |
| | ctgcagagcggcgtgcccagccggtttagcggcagcggctccggcaccgactt |
| | taccctgacaatttcctctctgcagcctgaggacttcgccacctactactgcc |
| | agcagtcttggagcgtgccccccttcaccttcggccagggcaccaaggtggag |
| | atcaagcgtacggtggctgcaccatctgtcttcatcttcccgccatctgatga |
| | gcagttgaaatctggaactgcctctgttgtgtgcctgctgaataacttctatc |
| | ccagagaggccaaagtacagtggaaggtggataacgccctccaatcgggtaac |
| | tcccaggagagtgtcacagagcaggacagcaaggacagcacctacagcctcag |
| | cagcaccctgacgctgagcaaagcagactacgagaaacacaaagtctacgcct |
| | gcgaagtcacccatcagggcctgagctcgcccgtcacaaagagcttcaacagg |
| | ggagagtgt |

MOR09610

| | |
|---|---|
| CDRH1 | 57 |
| | SYTFS |
| CDRH2 | 58 |
| | NILPIFGDANYAQKFQG |
| CDRH3 | 59 |
| | nkgafyymstypsldv |
| CDRL1 | 60 |
| | RASQNINYYLN |
| CDRL2 | 61 |
| | DAFSLQS |
| CDRL3 | 62 |
| | QQSIAVPPFT |
| VH | 63 |
| | evqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvss |
| VL | 64 |
| | DIQMTQSPSSLSASVGDRVTITCRASQNINYYLNWYQQKPGKAPKLLIYDAFS |
| | LQSGVPSRFSGSGSGTDFTLTISSLQPEDFATYYCQQSIAVPPFTFGQGTKVE |
| | IK |
| Heavy chain | 65 |
| | evqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvssastkgpsvfplapsskstsggtaalgclvkdyfp |
| | epvtvswnsgaltsgvhtfpavlqssglyslssvvtvpssslgtqtyicnvnh |
| | kpsntkvdkrvepkscdkthtcppcpapeaaggpsvflfppkpkdtlmisrtp |
| | evtcvvvdvshedpevkfnwyvdgvevhnaktkpreeqynstyrvvsvltvlh |
| | qdwlngkeykckvsnkalpapiektiskakgqprepqvytlppsreemtknqv |
| | sltclvkgfypsdiavewesngqpennykttppvldsdgsfflyskltvdksr |
| | wqqgnvfscsvmhealhnhytqkslslspgk |
| Light chain | 66 |
| | DIQMTQSPSSLSASVGDRVTITCRASQNINYYLNWYQQKPGKAPKLLIYDAFS |
| | LQSGVPSRFSGSGSGTDFTLTISSLQPEDFATYYCQQSIAVPPFTFGQGTKVE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC |
| PN encoding | 67 |
| SEQ.I.D.NO: 63 | gaggtgcaattggtgcagagcggagccgaagtgaagaagcccggcagcagcgt |
| | caaggtgtcctgcaaggccagcggcggcaccttcagcagctacaccttcagct |
| | gggtgcgccaggccccaggacagggcctggaatggatgggcaacatcctgccc |
| | atcttcggcgacgccaactacgcccagaagttccagggcagagtcaccatcac |
| | cgccgacgagagcaccagcaccgcctacatggaactgagcagcctgcggagcg |
| | aggacaccgccgtgtactactgcgcccggaacaagggcgccttctactacatg |
| | agcacctaccccagcctggacgtgtggggccagggcaccctggtgaccgtgag |
| | ctca |
| PN encoding | 68 |
| SEQ.I.D.NO: 64 | GATATCCAGATGACCCAGAGCCCCAGCAGCCTGAGCGCCAGCGTGGGCGACAG |
| | AGTGACCATCACCTGTCGGGCCAGCCAGAACATCAACTACTACCTGAACTGGT |
| | ATCAGCAGAAGCCCGGCAAGGCCCCCAAGCTGCTGATCTACGACGCCTTCAGC |
| | CTGCAGAGCGGCGTGCCCAGCCGGTTTAGCGGCAGCGGCTCCGGCACCGACTT |
| | TACCCTGACAATTTCCTCTCTGCAGCCTGAGGACTTCGCCACCTACTACTGCC |
| | AGCAGAGCATTGCCGTGCCCCCCTTCACCTTCGGCCAGGGCACCAAGGTGGAG |
| | ATCAAG |
| PN encoding | 69 |
| SEQ.I.D.NO: 65 | gaggtgcaattggtgcagagcggagccgaagtgaagaagcccggcagcagcgt |
| | caaggtgtcctgcaaggccagcggcggcaccttcagcagctacaccttcagct |
| | gggtgcgccaggccccaggacagggcctggaatggatgggcaacatcctgccc |
| | atcttcggcgacgccaactacgcccagaagttccagggcagagtcaccatcac |
| | cgccgacgagagcaccagcaccgcctacatggaactgagcagcctgcggagcg |
| | aggacaccgccgtgtactactgcgcccggaacaagggcgccttctactacatg |
| | agcacctaccccagcctggacgtgtggggccagggcaccctggtgaccgtgag |
| | ctcagcctccaccaagggtccatcggtcttccccctggcaccctcctccaaga |
| | gcacctctgggggcacagcggccctgggctgcctggtcaaggactacttcccc |
| | gaaccggtgacggtgtcgtggaactcaggcgccctgaccagcggcgtgcacac |
| | cttcccggctgtcctacagtcctcaggactctactccctcagcagcgtggtga |
| | ccgtgccctccagcagcttgggcacccagacctacatctgcaacgtgaatcac |
| | aagcccagcaacaccaaggtggacaagagagttgagcccaaatcttgtgacaa |
| | aactcacacatgcccaccgtgcccagcacctgaagcagcggggggaccgtcag |
| | tcttcctcttccccccaaaacccaaggacaccctcatgatctcccggacccct |
| | gaggtcacatgcgtggtggtggacgtgagccacgaagaccctgaggtcaagtt |
| | caactggtacgtggacggcgtggaggtgcataatgccaagacaaagccgcggg |
| | aggagcagtacaacagcacgtaccgggtggtcagcgtcctcaccgtcctgcac |
| | caggactggctgaatggcaaggagtacaagtgcaaggtctccaacaaagccct |
| | cccagcccccatcgagaaaaccatctccaaagccaaagggcagccccgagaac |
| | cacaggtgtacaccctgcccccatcccgggaggagatgaccaagaaccaggtc |
| | agcctgacctgcctggtcaaaggcttctatcccagcgacatcgccgtggagtg |
| | ggagagcaatgggcagccggagaacaactacaagaccacgcctcccgtgctgg |
| | actccgacggctccttcttcctctacagcaagctcaccgtggacaagagcagg |
| | tggcagcaggggaacgtcttctcatgctccgtgatgcatgaggctctgcacaa |
| | ccactacacgcagaagagcctctccctgtctccgggtaaa |
| PN encoding | 70 |
| SEQ.I.D.NO: 66 | GATATCCAGATGACCCAGAGCCCCAGCAGCCTGAGCGCCAGCGTGGGCGACAG |
| | AGTGACCATCACCTGTCGGGCCAGCCAGAACATCAACTACTACCTGAACTGGT |
| | ATCAGCAGAAGCCCGGCAAGGCCCCCAAGCTGCTGATCTACGACGCCTTCAGC |
| | CTGCAGAGCGGCGTGCCCAGCCGGTTTAGCGGCAGCGGCTCCGGCACCGACTT |
| | TACCCTGACAATTTCCTCTCTGCAGCCTGAGGACTTCGCCACCTACTACTGCC |
| | AGCAGAGCATTGCCGTGCCCCCCTTCACCTTCGGCCAGGGCACCAAGGTGGAG |
| | ATCAAGCGTACGGTGGCTGCACCATCTGTCTTCATCTTCCCGCCATCTGATGA |
| | GCAGTTGAAATCTGGAACTGCCTCTGTTGTGTGCCTGCTGAATAACTTCTATC |
| | CCAGAGAGGCCAAAGTACAGTGGAAGGTGGATAACGCCCTCCAATCGGGTAAC |
| | TCCCAGGAGAGTGTCACAGAGCAGGACAGCAAGGACAGCACCTACAGCCTCAG |
| | CAGCACCCTGACGCTGAGCAAAGCAGACTACGAGAAACACAAAGTCTACGCCT |
| | GCGAAGTCACCCATCAGGGCCTGAGCTCGCCCGTCACAAAGAGCTTCAACAGG |
| | GGAGAGTGT |

MOR09674

| | |
|---|---|
| CDRH1 | 71 |
| | SYSMH |
| CDRH2 | 72 |
| | LINPYNGNTHYAQKFQG |
| CDRH3 | 73 |
| | MLRFDV |
| CDRL1 | 74 |
| | TGTSSDGGGYNYVS |
| CDRL2 | 75 |
| | GVSNRPS |
| CDRL3 | 76 |
| | QTYTRYSDSPV |
| VH | 77 |
| | EVQLVQSGAEVKKPGASVKVSCKASGYTFTSYSMHWVRQAPGQGLEWMGLINP |
| | YNGNTHYAQKFQGRVTMTRDTSISTAYMELSSLRSEDTAVYYCARMLRFDVWG |
| | QGTLVTVSS |
| VL | 78 |
| | ESALTQPASVSGSPGQSITISCTGTSSDGGGYNYVSWYQQHPGKAPKLMIYGV |
| | SNRPSGVSNRFSGSKSGNTASLTISGLQAEDEADYYCQTYTRYSDSPVFGGGT |
| | KLTVLGQ |
| Heavy chain | 79 |
| | EVQLVQSGAEVKKPGASVKVSCKASGYTFTSYSMHWVRQAPGQGLEWMGLINP |
| | YNGNTHYAQKFQGRVTMTRDTSISTAYMELSSLRSEDTAVYYCARMLRFDVWG |
| | QGTLVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKR |
| | VEPKSCDKTHTCPPCPAPEAAGGPSVFLFPPKPKDTLMISRTPEVTCVVVDVS |
| | HEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFY |
| | PSDIAVEWESNGQPENNYKTTPPVLDSDGSFFLYSKLTVDKSRWQQGNVFSCS |
| | VMHEALHNHYTQKSLSLSPGK |
| Light chain | 80 |
| | ESALTQPASVSGSPGQSITISCTGTSSDGGGYNYVSWYQQHPGKAPKLMIYGV |
| | SNRPSGVSNRFSGSKSGNTASLTISGLQAEDEADYYCQTYTRYSDSPVFGGGT |
| | KLTVLGQPKAAPSVTLFPPSSEELQANKATLVCLISDFYPGAVTVAWKADSSP |
| | VKAGVETTTPSKQSNNKYAASSYLSLTPEQWKSHRSYSCQVTHEGSTVEKTVA |
| | PTECS |
| PN encoding | 81 |
| SEQ.I.D.NO: 77 | GAGGTGCAGCTGGTGCAGAGCGGAGCCGAAGTGAAGAAACCAGGCGCTTCCGT |
| | GAAGGTGTCCTGCAAGGCCAGCGGCTACACCTTCACCAGCTACAGCATGCACT |
| | GGGTCCGGCAGGCTCCAGGGCAGGGACTGGAATGGATGGGCCTGATCAACCCC |
| | TACAACGGCAACACCCACTACGCCCAGAAATTCCAGGGCAGAGTGACCATGAC |
| | CCGGGACACCAGCATCAGCACCGCCTACATGGAACTGAGCAGCCTGCGGAGCG |
| | AGGACACCGCCGTGTACTACTGCGCCCGGATGCTGCGGTTCGACGTGTGGGGC |
| | CAGGGCACCCTGGTCACCGTCAGCTCA |
| PN encoding | 82 |
| SEQ.I.D.NO: 78 | GAGAGCGCCCTGACCCAGCCTGCCAGCGTGTCTGGCAGCCCTGGCCAGAGCAT |
| | CACCATCAGCTGCACCGGCACCAGCAGCGACGGCGGAGGCTACAACTACGTGT |
| | CCTGGTATCAGCAGCACCCCGGCAAGGCCCCCAAGCTGATGATCTACGGCGTG |
| | AGCAACCGGCCCAGCGGGGTGTCCAACCGGTTCAGCGGCAGCAAGAGCGGCAA |
| | CACCGCCAGCCTGACCATCTCTGGGCTGCAGGCTGAGGACGAGGCCGACTACT |
| | ACTGCCAGACCTACACCAGATACAGCGACAGCCCTGTGTTCGGAGGCGGAACA |
| | AAGTTAACCGTCCTA |
| PN encoding | 83 |
| SEQ.I.D.NO: 79 | GAGGTGCAGCTGGTGCAGAGCGGAGCCGAAGTGAAGAAACCAGGCGCTTCCGT |
| | GAAGGTGTCCTGCAAGGCCAGCGGCTACACCTTCACCAGCTACAGCATGCACT |
| | GGGTCCGGCAGGCTCCAGGGCAGGGACTGGAATGGATGGGCCTGATCAACCCC |
| | TACAACGGCAACACCCACTACGCCCAGAAATTCCAGGGCAGAGTGACCATGAC |
| | CCGGGACACCAGCATCAGCACCGCCTACATGGAACTGAGCAGCCTGCGGAGCG |
| | AGGACACCGCCGTGTACTACTGCGCCCGGATGCTGCGGTTCGACGTGTGGGGC |
| | CAGGGCACCCTGGTCACCGTCAGCTCAGCCTCCACCAAGGGTCCATCGGTCTT |
| | CCCCCTGGCACCCTCCTCCAAGAGCACCTCTGGGGGCACAGCGGCCCTGGGCT |
| | GCCTGGTCAAGGACTACTTCCCCGAACCGGTGACGGTGTCGTGGAACTCAGGC |
| | GCCCTGACCAGCGGCGTGCACACCTTCCCGGCTGTCCTACAGTCCTCAGGACT |
| | CTACTCCCTCAGCAGCGTGGTGACCGTGCCCTCCAGCAGCTTGGGCACCCAGA |
| | CCTACATCTGCAACGTGAATCACAAGCCCAGCAACACCAAGGTGGACAAGAGA |
| | GTTGAGCCCAAATCTTGTGACAAAACTCACACATGCCCACCGTGCCCAGCACC |
| | TGAAGCAGCGGGGGGACCGTCAGTCTTCCTCTTCCCCCCAAAACCCAAGGACA |
| | CCCTCATGATCTCCCGGACCCCTGAGGTCACATGCGTGGTGGTGGACGTGAGC |
| | CACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGGGTGG |
| | TCAGCGTCCTCACCGTCCTGCACCAGGACTGGCTGAATGGCAAGGAGTACAAG |
| | TGCAAGGTCTCCAACAAAGCCCTCCCAGCCCCCATCGAGAAAACCATCTCCAA |
| | AGCCAAAGGGCAGCCCCGAGAACCACAGGTGTACACCCTGCCCCCATCCCGGG |
| | AGGAGATGACCAAGAACCAGGTCAGCCTGACCTGCCTGGTCAAAGGCTTCTAT |
| | CCCAGCGACATCGCCGTGGAGTGGGAGAGCAATGGGCAGCCGGAGAACAACTA |
| | CAAGACCACGCCTCCCGTGCTGGACTCCGACGGCTCCTTCTTCCTCTACAGCA |
| | AGCTCACCGTGGACAAGAGCAGGTGGCAGCAGGGGAACGTCTTCTCATGCTCC |
| | GTGATGCATGAGGCTCTGCACAACCACTACACGCAGAAGAGCCTCTCCCTGTC |
| | TCCGGGTAAA |
| PN encoding | 84 |
| SEQ.I.D.NO: 80 | GAGAGCGCCCTGACCCAGCCTGCCAGCGTGTCTGGCAGCCCTGGCCAGAGCAT |
| | CACCATCAGCTGCACCGGCACCAGCAGCGACGGCGGAGGCTACAACTACGTGT |
| | CCTGGTATCAGCAGCACCCCGGCAAGGCCCCCAAGCTGATGATCTACGGCGTG |
| | AGCAACCGGCCCAGCGGGGTGTCCAACCGGTTCAGCGGCAGCAAGAGCGGCAA |
| | CACCGCCAGCCTGACCATCTCTGGGCTGCAGGCTGAGGACGAGGCCGACTACT |
| | ACTGCCAGACCTACACCAGATACAGCGACAGCCCTGTGTTCGGAGGCGGAACA |
| | AAGTTAACCGTCCTAGGTCAGCCCAAGGCTGCCCCCTCGGTCACTCTGTTCCC |
| | GCCCTCCTCTGAGGAGCTTCAAGCCAACAAGGCCACACTGGTGTGTCTCATAA |
| | GTGACTTCTACCCGGGAGCCGTGACAGTGGCCTGGAAGGCAGATAGCAGCCCC |
| | GTCAAGGCGGGAGTGGAGACCACCACACCCTCCAAACAAAGCAACAACAAGTA |
| | CGCGGCCAGCAGCTATCTGAGCCTGACGCCTGAGCAGTGGAAGTCCCACAGAA |
| | GCTACAGCTGCCAGGTCACGCATGAAGGGAGCACCGTGGAGAAGACAGTGGCC |
| | CCTACAGAATGTTCA |

MOR09675

| | |
|---|---|
| CDRH1 | 85 |
| | GYYIN |
| CDRH2 | 86 |
| | IISPNQGTTGYAQKFQG |
| CDRH3 | 87 |
| | GNYDHLDY |
| CDRL1 | 88 |
| | RASQGISNYLN |
| CDRL2 | 89 |
| | DASTLQS |
| CDRL3 | 90 |
| | QQDWHTLPVT |
| VH | 91 |
| | EVQLVQSGAEVKKPGASVKVSCKASGYTFTGYYINWVRQAPGQGLEWMGIISP |
| | NQGTTGYAQKFQGRVTMTRDTSISTAYMELSSLRSEDTAVYYCARGNYDHLDY |
| | WGQGTLVTVSS |
| VL | 92 |
| | DIQMTQSPSSLSASVGDRVTITCRASQGISNYLNWYQQKPGKAPKLLIYDAST |
| | LQSGVPSRFSGSGSGTDFTLTISSLQPEDFATYYCQQDWHTLPVTFGQGTKVE |
| | IK |
| Heavy chain | 93 |
| | EVQLVQSGAEVKKPGASVKVSCKASGYTFTGYYINWVRQAPGQGLEWMGIISP |
| | NQGTTGYAQKFQGRVTMTRDTSISTAYMELSSLRSEDTAVYYCARGNYDHLDY |
| | WGQGTLVTVSSASTKGPSVFPLAPSSKSTSGGTAALGCLVKDYFPEPVTVSWN |
| | SGALTSGVHTFPAVIQSSGLYSLSSVVTVPSSSLGTQTYICNVNHKPSNTKVD |
| | KRVEPKSCDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKE |
| | YKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFS |
| | CSVMHEALHNHYTQKSLSLSPGK |
| Light chain | 94 |
| | DIQMTQSPSSLSASVGDRVTITCRASQGISNYLNWYQQKPGKAPKLLIYDAST |
| | LQSGVPSRFSGSGSGTDFTLTISSLQPEDFATYYCQQDWHTLPVTFGQGTKVE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC |
| PN encoding | 95 |
| SEQ.I.D.NO: 91 | GAGGTGCAGCTGGTGCAGAGCGGAGCCGAAGTGAAGAAACCAGGCGCTTCCGT |
| | GAAGGTGTCCTGCAAGGCCAGCGGCTACACCTTCACCGGCTACTACATCAACT |
| | GGGTCCGGCAGGCTCCAGGGCAGGGACTGGAATGGATGGGCATCATCAGCCCC |
| | AACCAGGGCACAACCGGCTACGCCCAGAAATTCCAGGGCAGAGTGACCATGAC |
| | CCGGGACACCAGCATCAGCACCGCCTACATGGAACTGAGCAGCCTGCGGAGCG |
| | AGGACACCGCCGTGTACTACTGCGCCAGAGGCAACTACGACCACCTGGACTAC |
| | TGGGGCCAGGGCACCCTGGTCACCGTCAGCTCA |
| PN encoding | 96 |
| SEQ.I.D.NO: 92 | GATATCCAGATGACCCAGAGCCCCAGCAGCCTGAGCGCCAGCGTGGGCGACAG |
| | AGTGACCATCACCTGTCGGGCCAGCCAGGGCATCAGCAACTACCTGAACTGGT |
| | ATCAGCAGAAGCCCGGCAAGGCCCCCAAGCTGCTGATCTACGACGCCAGCACC |
| | CTGCAGAGCGGCGTGCCTAGCAGATTCTCCGGGAGCGGCTCCGGCACCGACTT |
| | CACCCTGACCATTAGCTCACTGCAGCCAGAAGACTTCGCCACCTACTACTGCC |
| | AGCAGGACTGGCACACCCTGCCCGTGACCTTCGGCCAGGGCACCAAGGTGGAG |
| | ATCAAG |
| PN encoding | 97 |
| SEQ.I.D.NO: 93 | GAGGTGCAGCTGGTGCAGAGCGGAGCCGAAGTGAAGAAACCAGGCGCTTCCGT |
| | GAAGGTGTCCTGCAAGGCCAGCGGCTACACCTTCACCGGCTACTACATCAACT |
| | GGGTCCGGCAGGCTCCAGGGCAGGGACTGGAATGGATGGGCATCATCAGCCCC |
| | AACCAGGGCACAACCGGCTACGCCCAGAAATTCCAGGGCAGAGTGACCATGAC |
| | CCGGGACACCAGCATCAGCACCGCCTACATGGAACTGAGCAGCCTGCGGAGCG |
| | AGGACACCGCCGTGTACTACTGCGCCAGAGGCAACTACGACCACCTGGACTAC |
| | TGGGGCCAGGGCACCCTGGTCACCGTCAGCTCAGCCTCCACCAAGGGTCCATC |
| | GGTCTTCCCCCTGGCACCCTCCTCCAAGAGCACCTCTGGGGGCACAGCGGCCC |
| | TGGGCTGCCTGGTCAAGGACTACTTCCCCGAACCGGTGACGGTGTCGTGGAAC |
| | TCAGGCGCCCTGACCAGCGGCGTGCACACCTTCCCGGCTGTCCTACAGTCCTC |
| | AGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGAC |
| | AAGAGAGTTGAGCCCAAATCTTGTGACAAAACTCACACATGCCCACCGTGCCC |
| | AGCACCTGAAGCAGCGGGGGGACCGTCAGTCTTCCTCTTCCCCCCAAAACCCA |
| | AGGACACCCTCATGATCTCCCGGACCCCTGAGGTCACATGCGTGGTGGTGGAC |
| | GTGAGCCACGAAGACCCTGAGGTCAAGTTCAACTGGTACGTGGACGGCGTGGA |
| | GGTGCATAATGCCAAGACAAAGCCGCGGGAGGAGCAGTACAACAGCACGTACC |
| | GGGTGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCAT |
| | CCCGGGAGGAGATGACCAAGAACCAGGTCAGCCTGACCTGCCTGGTCAAAGGC |
| | TTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCT |
| | ACAGCAAGCTCACCGTGGACAAGAGCAGGTGGCAGCAGGGGAACGTCTTCTCA |
| | TGCTCCGTGATGCATGAGGCTCTGCACAACCACTACACGCAGAAGAGCCTCTC |
| | CCTGTCTCCGGGTAAA |
| PN encoding | 98 |
| SEQ.I.D.NO: 94 | GATATCCAGATGACCCAGAGCCCCAGCAGCCTGAGCGCCAGCGTGGGCGACAG |
| | AGTGACCATCACCTGTCGGGCCAGCCAGGGCATCAGCAACTACCTGAACTGGT |
| | ATCAGCAGAAGCCCGGCAAGGCCCCCAAGCTGCTGATCTACGACGCCAGCACC |
| | CTGCAGAGCGGCGTGCCTAGCAGATTCTCCGGGAGCGGCTCCGGCACCGACTT |
| | CACCCTGACCATTAGCTCACTGCAGCCAGAAGACTTCGCCACCTACTACTGCC |
| | AGCAGGACTGGCACACCCTGCCCGTGACCTTCGGCCAGGGCACCAAGGTGGAG |
| | ATCAAGCGTACGGTGGCTGCACCATCTGTCTTCATCTTCCCGCCATCTGATGA |
| | GCAGTTGAAATCTGGAACTGCCTCTGTTGTGTGCCTGCTGAATAACTTCTATC |
| | CCAGAGAGGCCAAAGTACAGTGGAAGGTGGATAACGCCCTCCAATCGGGTAAC |
| | TCCCAGGAGAGTGTCACAGAGCAGGACAGCAAGGACAGCACCTACAGCCTCAG |
| | CAGCACCCTGACGCTGAGCAAAGCAGACTACGAGAAACACAAAGTCTACGCCT |
| | GCGAAGTCACCCATCAGGGCCTGAGCTCGCCCGTCACAAAGAGCTTCAACAGG |
| | GGAGAGTGT |

MOR09124

| | |
|---|---|
| CDRH1 | 99 |
| | SYWMT |
| CDRH2 | 100 |
| | SIKIKPDGYAASVKG |
| CDRH3 | 101 |
| | LFYQYFARMDY |
| CDRL1 | 102 |
| | RASQDISNYLN |
| CDRL2 | 103 |
| | AASNLQS |
| CDRL3 | 104 |
| | QQYDSYSPT |
| VH | 105 |
| | qvqlvesggglvqpggslrlscaasgft fssywmtwvrqapgkglewvssiki |
| | kpdgyaasvkgrftisrdnskntlylqmnslraedtavyycarlfyqyfarmd |
| | ywgqgtlvtvss |
| VL | 106 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQYDSYSPTFGQGTKVEI |
| | K |
| Heavy chain | 107 |
| | qvqlvesggglvqpggslrlscaasgft fssywmtwvrqapgkglewvssiki |
| | kpdgyaasvkgrftisrdnskntlylqmnslraedtavyycarlfyqyfarmd |
| | ywgqgtlvtvssastkgpsvfplapsskstsggtaalgclvkdyfpepvtvsw |
| | nsgaltsgvhtfpavlqssglyslssvvtvpssslgtqtyicnvnhkpsntkv |
| | dkkvepks |
| Light chain | 108 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQYDSYSPTFGQGTKVEI |
| | KRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| PN encoding | 109 |
| SEQ.I.D.NO: 105 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctca |
| PN encoding | 110 |
| SEQ.I.D.NO: 106 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcggtttattattgcc |
| | agcagtatgattcttattctcctacctttggccagggtacgaaagttgaaatt |
| | aaa |
| PN encoding | 111 |
| SEQ.I.D.NO: 107 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctcagcgtcgaccaaaggtcc |
| | aagcgtgtttccgctggctccgagcagcaaaagcaccagcggcggcacggctg |
| | ccctgggctgcctggttaaagattatttcccggaaccagtcaccgtgagctgg |
| | aacagcggggcgctgaccagcggcgtgcatacctttccggcggtgctgcaaag |
| | cagcggcctgtatagcctgagcagcgttgtgaccgtgccgagcagcagcttag |
| | gcactcagacctatatttgcaacgtgaaccataaaccgagcaacaccaaagtg |
| | gataaaaaagtggaaccgaaaagc |
| PN encoding | 112 |
| SEQ.I.D.NO: 108 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcggtttattattgcc |
| | agcagtatgattcttattctcctacctttggccagggtacgaaagttgaaatt |
| | aaacgtacggtggctgctccgagcgtgtttatttttccgccgagcgatgaaca |
| | actgaaaagcggcacggcgagcgtggtgtgcctgctgaacaacttttatccgc |
| | gtgaagcgaaagttcagtggaaagtagacaacgcgctgcaaagcggcaacagc |
| | caggaaagcgtgaccgaacaggatagcaaagatagcacctattctctgagcag |
| | caccctgaccctgagcaaagcggattatgaaaaacataaagtgtatgcgtgcg |
| | aagtgacccatcaaggtctgagcagcccggtgactaaatcttttaatcgtggc |
| | gaggcc |

MOR09397

| | |
|---|---|
| CDRH1 | 113 |
| | SYWMT |
| CDRH2 | 114 |
| | SIKIKPDGYAASVKG |
| CDRH3 | 115 |
| | LFYQYFARMDY |
| CDRL1 | 116 |
| | RASQDISNYLN |
| CDRL2 | 117 |
| | AASNLQS |
| CDRL3 | 118 |
| | QQHDTFRPT |
| VH | 119 |
| | qvqlvesggglvqpggslrlscaasgftfssywmtwvrqapgkglewvssiki |
| | kpdgyaasvkgrftisrdnskntlylqmnslraedtavyycarlfyqyfarmd |
| | ywgqgtlvtvss |
| VL | 120 |
| | diqmtqspsslsasvgdrvtitcrasqdisnylnwyqqkpgkapklliyaasn |
| | lqsgvpsrfsgsgsgtdftltisslqpedfatyycqqhdtfrptfgqgtkvei |
| | k |
| Heavy chain | 121 |
| | qvqlvesggglvqpggslrlscaasgftfssywmtwvrqapgkglewvssiki |
| | kpdgyaasvkgrftisrdnskntlylqmnslraedtavyycarlfyqyfarmd |
| | ywgqgtlvtvssastkgpsvfplapsskstsggtaalgclvkdyfpepvtvsw |
| | nsgaltsgvhtfpavlqssglyslssvvtvpssslgtqtyicnvnhkpsntkv |
| | dkkvepks |
| Light chain | 122 |
| | diqmtqspsslsasvgdrvtitcrasqdisnylnwyqqkpgkapklliyaasn |
| | lqsgvpsrfsgsgsgtdftltisslqpedfatyycqqhdtfrptfgqgtkvei |
| | krtvaapsvfifppsdeqlksgtasvvcllnnfypreakvqwkvdnalqsgns |
| | qesvteqdskdstyslsstltlskadyekhkvyacevthqglsspvtksfnrg |
| | ec |
| PN encoding | 123 |
| SEQ.I.D.NO: 119 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctca |
| PN encoding | 124 |
| SEQ.I.D.NO: 120 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcgacctattattgcc |
| | agcagcatgatacttttcgtcctacctttggccagggtacgaaagttgaaatt |
| | aaa |
| PN encoding | 125 |
| SEQ.I.D.NO: 121 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctcagcgtcgaccaaaggtcc |
| | aagcgtgtttccgctggctccgagcagcaaaagcaccagcggcggcacggctg |
| | ccctgggctgcctggttaaagattatttcccggaaccagtcaccgtgagctgg |
| | aacagcggggcgctgaccagcggcgtgcatacctttccggcggtgctgcaaag |
| | cagcggcctgtatagcctgagcagcgttgtgaccgtgccgagcagcagcttag |
| | gcactcagacctatatttgcaacgtgaaccataaaccgagcaacaccaaagtg |
| | gataaaaaagtggaaccgaaaagc |
| PN encoding | 126 |
| SEQ.I.D.NO: 122 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcgacctattattgcc |
| | agcagcatgatacttttcgtcctacctttggccagggtacgaaagttgaaatt |
| | aaacgtacggtggctgctccgagcgtgtttatttttccgccgagcgatgaaca |
| | actgaaaagcggcacggcgagcgtggtgtgcctgctgaacaacttttatccgc |
| | gtgaagcgaaagttcagtggaaagtagacaacgcgctgcaaagcggcaacagc |
| | caggaaagcgtgaccgaacaggatagcaaagatagcacctattctctgagcag |
| | caccctgaccctgagcaaagcggattatgaaaaacataaagtgtatgcgtgcg |
| | aagtgacccatcaaggtctgagcagcccggtgactaaatcttttaatcgtggc |
| | gaggcc |

MOR09398

| | |
|---|---|
| CDRH1 | 127 |
| | SYWMT |
| CDRH2 | 128 |
| | SIKIKPDGYAASVKG |
| CDRH3 | 129 |
| | LFYQYFARMDY |
| CDRL1 | 130 |
| | RASQDISNYLN |
| CDRL2 | 131 |
| | AASNLQS |
| CDRL3 | 132 |
| | QQWDSFSPT |
| VH | 133 |
| | qvqlvesggglvqpggslrlscaasgftfssywmtwvrqapgkglewvssiki |
| | kpdgyaasvkgrftisrdnskntlylqmnslraedtavyycarlfyqyfarmd |
| | ywgqgtlvtvss |
| VL | 134 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQWDSFSPTFGQGTKVEI |
| | K |
| Heavy chain | 135 |
| | qvqlvesggglvqpggslrlscaasgftfssywmtwvrqapgkglewvssiki |
| | kpdgyaasvkgrftisrdnskntlylqmnslraedtavyycarlfyqyfarmd |
| | ywgqgtlvtvssastkgpsvfplapsskstsggtaalgclvkdyfpepvtvsw |
| | nsgaltsgvhtfpavlqssglyslssvvtvpssslgtqtyicnvnhkpsntkv |
| | dkkvepks |
| Light chain | 136 |
| | DIQMTQSPSSLSASVGDRVTITCRASQDISNYLNWYQQKPGKAPKLLIYAASN |
| | LQSGVPSRFSGSGSGTDFTLTISSLQPEDFATYYCQQWDSFSPTFGQGTKVEI |
| | KRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC |
| PN encoding | 137 |
| SEQ.I.D.NO: 133 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctca |
| PN encoding | 138 |
| SEQ.I.D.NO: 134 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcgacctattattgcc |
| | agcagtgggattctttttctcctacctttggccagggtacgaaagttgaaatt |
| | aaa |
| PN encoding | 139 |
| SEQ.I.D.NO: 135 | caggtgcaattggtggaaagcggcggcggcctggtgcaaccgggcggcagcct |
| | gcgtctgagctgcgcggcctccggatttaccttttcttcttattggatgactt |
| | gggtgcgccaagcccctgggaagggtctcgagtgggtgagctctattaagatt |
| | aagcctgatggttatgctgcttctgttaagggtcgttttaccatttcacgtga |
| | taattcgaaaaacaccctgtatctgcaaatgaacagcctgcgtgcggaagata |
| | cggccgtgtattattgcgcgcgtcttttttatcagtattttgctcgtatggat |
| | tattggggccaaggcaccctggtgacggttagctcagcgtcgaccaaaggtcc |
| | aagcgtgtttccgctggctccgagcagcaaaagcaccagcggcggcacggctg |
| | ccctgggctgcctggttaaagattatttcccggaaccagtcaccgtgagctgg |
| | aacagcggggcgctgaccagcggcgtgcatacctttccggcggtgctgcaaag |
| | cagcggcctgtatagcctgagcagcgtcgtgaccgtgccgagcagcagcttag |
| | gcactcagacctatatttgcaacgtgaaccataaaccgagcaacaccaaagtg |
| | gataaaaaagtggaaccgaaaagc |
| PN ancoding | 140 |
| SEQ.I.D.NO: 136 | gatatccagatgacccagagcccgtctagcctgagcgcgagcgtgggtgatcg |
| | tgtgaccattacctgcagagcgagccaggatatttctaattatctgaattggt |
| | accagcagaaaccaggtaaagcaccgaaactattaatttatgctgcttctaat |
| | ttgcaaagcggggtcccgtcccgttttagcggctctggatccggcactgattt |
| | taccctgaccattagcagcctgcaacctgaagactttgcgacctattattgcc |
| | agcagtgggattctttttctcctacctttggccagggtacgaaagttgaaatt |
| | aaacgtacggtggctgctccgagcgtgtttatttttccgccgagcgatgaaca |
| | actgaaaagcggcacggcgagcgtggtgtgcctgctgaacaacttttatccgc |
| | gtgaagcgaaagttcagtggaaagtagacaacgcgctgcaaagcggcaacagc |
| | caggaaagcgtgaccgaacaggatagcaaagatagcacctattctctgagcag |
| | caccctgaccctgagcaaagcggattatgaaaaacataaagtgtatgcgtgcg |
| | aagtgacccatcaaggtctgagcagcccggtgactaaatcttttaatcgtggc |
| | gaggcc |

MOR09136

| | |
|---|---|
| CDRH1 | 141 |
| | SYTFS |
| CDRH2 | 142 |
| | NILPIFGDANYAQKFQG |
| CDRH3 | 143 |
| | nkgafyymstypsldv |
| CDRL1 | 144 |
| | RASQNINYYLN |
| CDRL2 | 145 |
| | DAFSLQS |
| CDRL3 | 146 |
| | QQSWSVPPFT |
| VH | 147 |
| | qvqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvss |
| VL | 148 |
| | diqmtqspsslsasvgdrvtitcrasqninyylnwyqqkpgkapklliydafs |
| | lqsgvpsrfsgsgsgtdftltisslqpedfatyycqqswsvppftfgqgtkve |
| | ik |
| Heavy chain | 149 |
| | qvqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvssastkgpsvfplapsskstsggtaalgclvkdyfp |
| | epvtvswnsgaltsgvhtfpavlqssglyslssvvtvpssslgtqtyicnvnh |
| | kpsntkvdkkvepks |
| Light chain | 150 |
| | diqmtqspsslsasvgdrvtitcrasqninyylnwyqqkpgkapklliydafs |
| | lqsgvpsrfsgsgsgtdftltisslqpedfatyycqqswsvppftfgqgtkve |
| | ikrtvaapsvfifppsdeqlksgtasvvcllnnfypreakvqwkvdnalqsgn |
| | sqesvteqdskdstyslsstltlskadyekhkvyacevthqglsspvtksfnr |
| | gec |
| PN encoding | 151 |
| SEQ.I.D.NO: 147 | caggtgcaattggttcagtctggcgcggaagtgaaaaaaccgggcagcagcgt |
| | gaaagtgagctgcaaagcctccggaggcactttttcttcttatactttttctt |
| | gggtgcgccaagcccctgggcagggtctcgagtggatgggcaatatccttccg |
| | atttttggcgatgcgaattacgcgcagaagtttcagggccgggtgaccattac |
| | cgcggatgaaagcaccagcaccgcgtatatggaactgagcagcctgcgtagcg |
| | aagatacggccgtgtattattgcgcgcgtaataagggtgctttttattatatg |
| | tctacttatccttctcttgatgtttggggccaaggcaccctggtgacggttag |
| | ctca |
| PN encoding | 152 |
| SEQ.I.D.NO: 148 | gatatccagatgacccagagcccgtctagcctgagcgcgagcgtgggtgatcg |
| | tgtgaccattacctgcagagcgagccagaatattaattattatctgaattggt |
| | accagcagaaaccaggtaaagcaccgaaactattaatttatgatgctttttct |
| | ttgcaaagcggggtcccgtcccgttttagcggctctggatccggcactgattt |
| | taccctgaccattagcagcctgcaacctgaagactttgcgacctattattgcc |
| | agcagtcttggtctgttcctccttttacctttggccagggtacgaaagttgaa |
| | attaaa |
| PN encoding | 153 |
| SEQ.I.D.NO: 149 | caggtgcaattggttcagtctggcgcggaagtgaaaaaaccgggcagcagcgt |
| | gaaagtgagctgcaaagcctccggaggcactttttcttcttatactttttctt |
| | gggtgcgccaagcccctgggcagggtctcgagtggatgggcaatatccttccg |
| | atttttggcgatgcgaattacgcgcagaagtttcagggccgggtgaccattac |
| | cgcggatgaaagcaccagcaccgcgtatatggaactgagcagcctgcgtagcg |
| | aagatacggccgtgtattattgcgcgcgtaataagggtgctttttattatatg |
| | tctacttatccttctcttgatgtttggggccaaggcaccctggtgacggttag |
| | ctcagcgtcgaccaaaggtccaagcgtgtttccgctggctccgagcagcaaaa |
| | gcaccagcggcggcacggctgccctgggctgcctggttaaagattatttcccg |
| | gaaccagtcaccgtgagctggaacagcggggcgctgaccagcggcgtgcatac |
| | ctttccggcggtgctgcaaagcagcggcctgtatagcctgagcagcgttgtga |
| | ccgtgccgagcagcagcttaggcactcagacctatatttgcaacgtgaaccat |
| | aaaccgagcaacaccaaagtggataaaaaagtggaaccgaaaagc |
| PN encoding | 154 |
| SEQ.I.D.NO: 150 | gatatccagatgacccagagcccgtctagcctgagcgcgagcgtgggtgatcg |
| | tgtgaccattacctgcagagcgagccagaatattaattattatctgaattggt |
| | accagcagaaaccaggtaaagcaccgaaactattaatttatgatgctttttct |
| | ttgcaaagcggggtcccgtcccgttttagcggctctggatccggcactgattt |
| | taccctgaccattagcagcctgcaacctgaagactttgcgacctattattgcc |
| | agcagtcttggtctgttcctccttttacctttggccagggtacgaaagttgaa |
| | attaaacgtacggtggctgctccgagcgtgtttatttttccgccgagcgatga |
| | acaactgaaaagcggcacggcgagcgtggtgtgcctgctgaacaacttttatc |
| | cgcgtgaagcgaaagttcagtggaaagtagacaacgcgctgcaaagcggcaac |
| | agccaggaaagcgtgaccgaacaggatagcaaagatagcacctattctctgag |
| | cagcaccctgaccctgagcaaagcggattatgaaaaacataaagtgtatgcgt |
| | gcgaagtgacccatcaaggtctgagcagcccggtgactaaatcttttaatcgt |
| | ggcgaggcc |

MOR09141

| | |
|---|---|
| CDRH1 | 155 |
| | SYTFS |
| CDRH2 | 156 |
| | NILPIFGDANYAQKFQG |
| CDRH3 | 157 |
| | nkgafyymstypldv |
| CDRL1 | 158 |
| | RASQNINYYLN |
| CDRL2 | 159 |
| | DAFSLQS |
| CDRL3 | 160 |
| | QQSIAVPPFT |
| VH | 161 |
| | qvqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvss |
| VL | 162 |
| | DIQMTQSPSSLSASVGDRVTITCRASQNINYYLNWYQQKPGKAPKLLIYDAFS |
| | LQSGVPSRFSGSGSGTDFTLTISSLQPEDFATYYCQQSIAVPPFTFGQGTKVE |
| | IK |
| Heavy chain | 163 |
| | qvqlvqsgaevkkpgssvkvsckasggtfssytfswvrqapgqglewmgnilp |
| | ifgdanyaqkfqgrvtitadeststaymelsslrsedtavyycarnkgafyym |
| | stypsldvwgqgtlvtvssastkgpsvfplapsskstsggtaalgclvkdyfp |
| | epvtvswnsgaltsgvhtfpavlqssglyslssvvtvpssslgtqtyicnvnh |
| | kpsntkvdkkvepks |
| Light chain | 164 |
| | DIQMTQSPSSLSASVGDRVTITCRASQNINYYLNWYQQKPGKAPKLLIYDAFS |
| | LQSGVPSRFSGSGSGTDFTLTISSLQPEDFATYYCQQSIAVPPFTFGQGTKVE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC |
| PN encoding | 165 |
| SEQ.I.D.NO: 161 | caggtgcaattggttcagtctggcgcggaagtgaaaaaaccgggcagcagcgt |
| | gaaagtgagctgcaaagcctccggaggcactttttcttcttatactttttctt |
| | gggtgcgccaagcccctgggcagggtctcgagtggatgggcaatatccttccg |
| | atttttggcgatgcgaattacgcgcagaagtttcagggccgggtgaccattac |
| | cgcggatgaaagcaccagcaccgcgtatatggaactgagcagcctgcgtagcg |
| | aagatacggccgtgtattattgcgcgcgtaataagggtgctttttattatatg |
| | tctacttatccttctcttgatgtttggggccaaggcaccctggtgacggttag |
| | ctca |
| PN encoding | 166 |
| SEQ.I.D.NO: 162 | gatatccagatgacccagagcccgtctagcctgagcgcgagcgtgggtgatcg |
| | tgtgaccattacctgcagagcgagccagaatattaattattatctgaattggt |
| | accagcagaaaccaggtaaagcaccgaaactattaatttatgatgctttttct |
| | ttgcaaagcggggtcccgtcccgttttagcggctctggatccggcactgattt |
| | taccctgaccattagcagcctgcaacctgaagactttgcgacctattattgcc |
| | agcagtctattgctgttcctccttttacctttggccagggtacgaaagttgaa |
| | attaaa |
| PN encoding | 167 |
| SEQ.I.D.NO: 163 | caggtgcaattggttcagtctggcgcggaagtgaaaaaaccgggcagcagcgt |
| | gaaagtgagctgcaaagcctccggaggcactttttcttcttatactttttctt |
| | gggtgcgccaagcccctgggcagggtctcgagtggatgggcaatatccttccg |
| | atttttggcgatgcgaattacgcgcagaagtttcagggccgggtgaccattac |
| | cgcggatgaaagcaccagcaccgcgtatatggaactgagcagcctgcgtagcg |
| | aagatacggccgtgtattattgcgcgcgtaataagggtgctttttattatatg |
| | tctacttatccttctcttgatgtttggggccaaggcaccctggtgacggttag |
| | ctcagcgtcgaccaaaggtccaagcgtgtttccgctggctccgagcagcaaaa |
| | gcaccagcggcggcacggctgccctgggctgcctggttaaagattatttcccg |
| | gaaccagtcaccgtgagctggaacagcggggcgctgaccagcggcgtgcatac |
| | ctttccggcggtgctgcaaagcagcggcctgtatagcctgagcagcgttgtga |
| | ccgtgccgagcagcagcttaggcactcagacctatatttgcaacgtgaaccat |
| | aaaccgagcaacaccaaagtggataaaaaagtggaaccgaaaagc |
| PN encoding | 168 |
| SEQ.I.D.NO: 164 | gatatccagatgacccagagcccgtctagcctgagcgcgagcgtgggtgatcg |
| | tgtgaccattacctgcagagcgagccagaatattaattattatctgaattggt |
| | accagcagaaaccaggtaaagcaccgaaactattaatttatgatgctttttct |
| | ttgcaaagcggggtcccgtcccgttttagcggctctggatccggcactgattt |
| | taccctgaccattagcagcctgcaacctgaagactttgcgacctattattgcc |
| | agcagtctattgctgttcctccttttacctttggccagggtacgaaagttgaa |
| | attaaacgtacggtggctgctccgagcgtgtttatttttccgccgagcgatga |
| | acaactgaaaagcggcacggcgagcgtggtgtgcctgctgaacaacttttatc |
| | cgcgtgaagcgaaagttcagtggaaagtagacaacgcgctgcaaagcggcaac |
| | agccaggaaagcgtgaccgaacaggatagcaaagatagcacctattctctgag |
| | cagcaccctgaccctgagcaaagcggattatgaaaaacataaagtgtatgcgt |
| | gcgaagtgacccatcaaggtctgagcagcccggtgactaaatcttttaatcgt |
| | ggcgaggcc |

MOR09373

| | |
|---|---|
| CDRH1 | 169 |
| | SYSMH |
| CDRH2 | 170 |
| | LINPYNGNTHYAQKFQG |
| CDRH3 | 171 |
| | MLRFDV |
| CDRL1 | 172 |
| | TGTSSDGGGYNYVS |
| CDRL2 | 173 |
| | GVSNRPS |
| CDRL3 | 174 |
| | QTYTRYSDSPV |
| VH | 175 |
| | qvqlvqsgaevkkpgasvkvsckasgytftsysmhwvrqapgqglewmglinp |
| | yngnthyaqkfqgrvtmtrdtsistaymelsslrsedtavyycarmlrfdvwg |
| | qgtlvtvss |
| VL | 176 |
| | dialtqpasvsgspgqsitisctgtssdgggynyvswyqqhpgkapklmiygv |
| | snrpsgvsnrfsgsksgntasltisglqaedeadyycqtytrysdspvfgggt |
| | kltvl |
| Heavy chain | 177 |
| | qvqlvqsgaevkkpgasvkvsckasgytftsysmhwvrqapgqglewmglinp |
| | yngnthyaqkfqgrvtmtrdtsistaymelsslrsedtavyycarmlrfdvwg |
| | qgtlvtvssastkgpsvfplapsskstsggtaalgclvkdyfpepvtvswnsg |
| | altsgvhtfpavlqssglyslssvvtvpssslgtqtyicnvnhkpsntkvdkk |
| | vepks |
| Light chain | 178 |
| | dialtqpasvsgspgqsitisctgtssdgggynyvswyqqhpgkapklmiygv |
| | snrpsgvsnrfsgsksgntasltisglqaedeadyycqtytrysdspvfgggt |
| | kltvlgqpkaapsvtlfppsseelqankatlvclisdfypgavtvawkadssp |
| | vkagvetttpskqsnnkyaassylsltpeqwkshrsyscqvthegstvektva |
| | ptea |
| PN encoding | 179 |
| SEQ.I.D.NO: 175 | caggtgcaattggttcagagcggcgcggaagtgaaaaaaccgggcgcgagcgt |
| | gaaagtgagctgcaaagcctccggatatacctttacttcttattctatgcatt |
| | gggtccgccaagcccctgggcagggtctcgagtggatgggccttatcaatccg |
| | tataatggcaatacgcattacgcgcagaagtttcagggccgggtgaccatgac |
| | ccgtgataccagcattagcaccgcgtatatggaactgagcagcctgcgtagcg |
| | aagatacggccgtgtattattgcgcgcgtatgcttcgttttgatgtttggggc |
| | caaggcaccctggtgacggttagctca |
| PN encoding | 180 |
| SEQ.I.D.NO: 176 | gatatcgcactgacccagccagcttcagtgagcggctcaccaggtcagagcat |
| | taccatctcgtgtacgggtactagcagcgatggtggtggttataattatgtgt |
| | cttggtaccagcagcatcccgggaaggcgccgaaacttatgatttatggtgtt |
| | tctaatcgtccctcaggcgtgagcaaccgttttagcggatccaaaagcggcaa |
| | caccgcgagcctgaccattagcggcctgcaagcggaagacgaagcggattatt |
| | attgccagacttatactcgttattctgattctcctgtgtttggcggcggcacg |
| | aagttaaccgttctt |
| PN encoding | 181 |
| SEQ.I.D.NO: 177 | caggtgcaattggttcagagcggcgcggaagtgaaaaaaccgggcgcgagcgt |
| | gaaagtgagctgcaaagcctccggatatacctttacttcttattctatgcatt |
| | gggtccgccaagcccctgggcagggtctcgagtggatgggccttatcaatccg |
| | tataatggcaatacgcattacgcgcagaagtttcagggccgggtgaccatgac |
| | ccgtgataccagcattagcaccgcgtatatggaactgagcagcctgcgtagcg |
| | aagatacggccgtgtattattgcgcgcgtatgcttcgttttgatgtttggggc |
| | caaggcaccctggtgacggttagctcagcgtcgaccaaaggtccaagcgtgtt |
| | tccgctggctccgagcagcaaaagcaccagcggcggcacggctgccctgggct |
| | gcctggttaaagattatttcccggaaccagtcaccgtgagctggaacagcggg |
| | gcgctgaccagcggcgtgcatacctttccggcggtgctgcaaagcagcggcct |
| | gtatagcctgagcagcgttgtgaccgtgccgagcagcagcttaggcactcaga |
| | cctatatttgcaacgtgaaccataaaccgagcaacaccaaagtggataaaaaa |
| | gtggaaccgaaaagc |
| PN encoding | 182 |
| SEQ.I.D.NO: 178 | gatatcgcactgacccagccagcttcagtgagcggctcaccaggtcagagcat |
| | taccatctcgtgtacgggtactagcagcgatggtggtggttataattatgtgt |
| | cttggtaccagcagcatcccgggaaggcgccgaaacttatgatttatggtgtt |
| | tctaatcgtccctcaggcgtgagcaaccgttttagcggatccaaaagcggcaa |
| | caccgcgagcctgaccattagcggcctgcaagcggaagacgaagcggattatt |
| | attgccagacttatactcgttattctgattctcctgtgtttggcggcggcacg |
| | aagttaaccgttcttggccagccgaaagccgcaccgagtgtgacgctgtttcc |
| | gccgagcagcgaagaattgcaggcgaacaaagcgaccctggtgtgcctgatta |
| | gcgacttttatccgggagccgtgacagtggcctggaaggcagatagcagcccc |
| | gtcaaggcgggagtggagaccaccacaccctccaaacaaagcaacaacaagta |
| | cgcggccagcagctatctgagcctgacgcctgagcagtggaagtcccacagaa |
| | gctacagctgccaggtcacgcatgaggggagcaccgtggaaaaaaccgttgcg |
| | ccgactgaggcc |

MOR09423

| | |
|---|---|
| CDRH1 | 183 |
| | GYYIN |
| CDRH2 | 184 |
| | IISPNQGTTGYAQKFQG |
| CDRH3 | 185 |
| | GNYDHLDY |
| CDRL1 | 186 |
| | RASQGISNYLN |
| CDRL2 | 187 |
| | DASTLQS |
| CDRL3 | 188 |
| | QQDWHTLPVT |
| VH | 189 |
| | qvqlvqsgaevkkpgasvkvsckasgytftgyyinwvrqapgqglewmgiisp |
| | nqgttgyaqkfqgrvtmtrdtsistaymelsslrsedtavyycargnydhldy |
| | wgqgtlvtvss |
| VL | 190 |
| | DIQMTQSPSSLSASVGDRVTITCRASQGISNYLNWYQQKPGKAPKLLIYDAST |
| | LQSGVPSRFSGSGSGTDFTLTISSLQPEDFATYYCQQDWHTLPVTFGQGTKVE |
| | IK |
| Heavy chain | 191 |
| | qvqlvqsgaavkkpgasvkvsckasgytftgyyinwvrqapgqglawmgiisp |
| | nqgttgyaqkfqgrvtmtrdtsistaymalsslrsadtavyycargnydhldy |
| | wgqgtlvtvssastkgpsvfplapsskstsggtaalgclvkdyfpapvtvswn |
| | sgaltsgvhtfpavlqssglyslssvvtvpssslgtqtyicnvnhkpsntkvd |
| | kkvapks |
| Light chain | 192 |
| | DIQMTQSPSSLSASVGDRVTITCRASQGISNYLNWYQQKPGKAPKLLIYDAST |
| | LQSGVPSRFSGSGSGTDFTLTISSLQPEDFATYYCQQDWHTLPVTFGQGTKVE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC |
| PN encoding | 193 |
| SEQ.I.D.NO: 189 | caggtgcaattggttcagagcggcgcggaagtgaaaaaaccgggcgcgagcgt |
| | gaaagtgagctgcaaagcctccggatatacctttactggttattatattaatt |
| | gggtccgccaagcccctgggcagggtctcgagtggatgggcattatctctccg |
| | aatcagggcactacgggttacgcgcagaagtttcagggccgggtgaccatgac |
| | ccgtgataccagcattagcaccgcgtatatggaactgagcagcctgcgtagcg |
| | aagatacggccgtgtattattgcgcgcgtggtaattatgatcatcttgattat |
| | tggggccaaggcaccctggtgacggttagctca |
| PN encoding | 194 |
| SEQ.I.D.NO: 190 | gatatccagatgacccagagcccgtctagcctgagcgcgagcgtgggtgatcg |
| | tgtgaccattacctgcagagcgagccagggtatttctaattatctgaattggt |
| | accagcagaaaccaggtaaagcaccgaaactattaatttatgatgcttctact |
| | ttgcaaagcggggtcccgtcccgttttagcggctctggatccggcactgattt |
| | taccctgaccattagcagcctgcaacctgaagactttgcgacttattattgcc |
| | agcaggattggcatactcttcctgttacctttggccagggtacgaaagttgaa |
| | attaaa |
| PN encoding | 195 |
| SEQ.I.D.NO: 191 | caggtgcaattggttcagagcggcgcggaagtgaaaaaaccgggcgcgagcgt |
| | gaaagtgagctgcaaagcctccggatatacctttactggttattatattaatt |
| | gggtccgccaagcccctgggcagggtctcgagtggatgggcattatctctccg |
| | aatcagggcactacgggttacgcgcagaagtttcagggccgggtgaccatgac |
| | ccgtgataccagcattagcaccgcgtatatggaactgagcagcctgcgtagcg |
| | aagatacggccgtgtattattgcgcgcgtggtaattatgetcatcttgattat |
| | tggggccaaggcaccctggtgacggttagctcagcgtcgaccaaaggtccaag |
| | cgtgtttccgctggctccgagcagcaaaagcaccagcggcggcacggctgccc |
| | tgggctgcctggttaaagattatttcccggaaccagtcaccgtgagctggaac |
| | agcggggcgctgaccagcggcgtgcatacctttccggcggtgctgcaaagcag |
| | cggcctgtatagcctgagcagcgttgtgaccgtgccgagcagcagcttaggca |
| | ctcagacctatatttgcaacgtgaaccataaaccgagcaacaccaaagtggat |
| | aaaaaagtggaaccgaaaagc |
| PN encoding | 196 |
| SEQ.I.D.NO: 192 | gatatccagatgacccagagcccgtctagcctgagcgcgagcgtgggtgatcg |
| | tgtgaccattacctgcagagcgagccagggtatttctaattatctgaattggt |
| | accagcagaaaccaggtaaagcaccgaaactattaatttatgatgcttctact |
| | ttgcaaagcggggtcccgtcccgttttagcggctctggatccggcactgattt |
| | taccctgaccattagcagcctgcaacctgaagactttgcgacttattattgcc |
| | agcaggattggcatactcttcctgttacctttggccagggtacgaaagttgaa |
| | attaaacgtacggtggctgctccgagcgtgtttatttttccgccgagcgatga |
| | acaactgaaaagcggcacggcgagcgtggtgtgcctgctgaacaacttttatc |
| | cgcgtgaagcgaaagttcagtggaaagtagacaacgcgctgcaaagcggcaac |
| | agccaggaaagcgtgaccgaacaggatagcaaagatagcacctattctctgag |
| | cagcaccctgaccctgagcaaagcggattatgaaaaacataaagtgtatgcgt |
| | gcgaagtgacccatcaaggtctgagcagcccggtgactaaatcttttaatcgt |
| | ggcgaggcc |
| Human C3b A | 197 |
| Chain | SPMYSIITPNILRLESEETMVLEAHDAQGDVPVTVTVHDFPGKKLVLSSEKTV |
| | LTPATNHMGNVTFTIPANREFKSEKGRNKFVTVQATFGTQVVEKVVLVSLQSG |
| | YLFIQTDKTIYTPGSTVLYRIFTVNHKLLPVGRTVMVNIENPEGIPVKQDSLS |
| | SQNQLGVLPLSWDIPELVNMGQWKIRAYYENSPQQVFSTEFEVKEYVLPSFEV |
| | IVEPTEKFYYIYNEKGLEVTITARFLYGKKVEGTAFVIFGIQDGEQRISLPES |
| | LKRIPIEDGSGEVVLSRKVLLDGVQNLRAEDLVGKSLYVSATVILHSGSDMVQ |
| | AERSGIPIVTSPYQIHFTKTPKYFKPGMPFDLMVFVTNPDGSPAYRVPVAVQG |
| | EDTVQSLTQGDGVAKLSINTHPSQKPLSITVRTKKQELSEAEQATRTMQALPY |
| | STVGNSNNYLHLSVLRTELRPGETLNVNFLLRMDRAHEAKIRYYTYLIMNKGR |
| | LLKAGRQVREPGQDLVVLPLSITTDFIPSFRLVAYYTLIGASGQREVVADSVW |
| | VDVKDSCVGSLVVKSGQSEDRQPVPGQQMTLKIEGDHGARVVLVAVDKGVFVL |
| | NKKNKLTQSKIWDVVEKADIGCTPGSGKDYAGVFSDAGLTFTSSSGQQTAQRA |
| | ELQCPQPAA |
| Human C3b B | 198 |
| Chain | SNLDEDIIAEENIVSRSEFPESWLWNVEDLKEPPKNGISTKLMNIFLKDSITT |
| | WEILAVSMSDKKGICVADPFEVTVMQDFFIDLRLPYSVVRNEQVEIRAVLYNY |
| | RQNQELKVRVELLHNPAFCSLATTKRRHQQTVTIPPKSSLSVPYVIVPLKTGL |
| | QEVEVKAAVYHHFISDGVRKSLKVVPEGIRMNKTVAVRTLDPERLGREGVQKE |
| | DIPPADLSDQVPDTESETRILLQGTPVAQMTEDAVDAERLKHLIVTPSGCGEQ |
| | NMIGMTPTVIAVHYLDETEQWEKFGLEKRQGALELIKKGYTQQLAFRQPSSAF |
| | AAFVKRAPSTWLTAYVVKVFSLAVNLIAIDSQVLCGAVKWLILEKQKPDGVFQ |
| | EDAPVIHQEMIGGLRNNNEKDMALTAFVLISLQEAKDICEEQVNSLPGSITKA |
| | GDFLEANYMNLQRSYTVAIAGYALAQMGRLKGPLLNKFLTTAKDKNRWEDPGK |
| | QLYNVEATSYALLALLQLKDFDFVPPVVRWLNEQRYYGGGYGSTQATFMVFQA |
| | LAQYQKDAPDHQELNLDVSLQLPSRSSKITHRIHWESASLLRSEETKENEGFT |
| | VTAEGKGQGTLSVVTMYHAKAKDQLTCNKFDLKVTIKPAPETEKRPQDAKNTM |
| | ILEICTRYRGDQDATMSILDISMMTGFAPDTDDLKQLANGVDRYISKYELDKA |
| | FSDRNTLIIYLDKVSHSEDDCLAFKVHQYFNVELIQPGAVKVYAYYNLEESCT |
| | RFYHPEKEDGKLNKLCRDELCRCAEENCFIQKSDDKVTLEERLDKACEPGVDY |
| | VYKTRLVKVQLSNDFDEYIMAIEQTIKSGSDEVQVGQQRTFISPIKCREALKL |
| | EEKKHYLMWGLSSDFWGEKPNLSYIIGKDTWVEHWPEEDECQDEENQKQCQDL |
| | GAFTESMVVFGCPN |

Other antibodies of the invention include those where the amino acids or nucleic acids encoding the amino acids have been mutated, yet have at least 60, 65, 70, 75, 80, 85, 90, or 95 percent identity to the sequences described in Table 1. In some embodiments, it includes mutant amino acid sequences wherein no more than 1, 2, 3, 4 or 5 amino acids have been mutated in the variable regions when compared with the variable regions depicted in the sequence described in Table 1, while retaining substantially the same therapeutic activity.

Since each of these antibodies can bind to C3b, the VH, VL, full length light chain, and full length heavy chain sequences (amino acid sequences and the nucleotide sequences encoding the amino acid sequences) can be “mixed and matched” to create other C3b-binding antibodies of the invention. Such “mixed and matched” C3b-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence. Accordingly, in one aspect, the invention provides an isolated monoclonal antibody or antigen binding region thereof having: a heavy chain variable domain comprising an amino acid sequence selected from the group consisting of SEQ ID NOs: 7, 21, 35, 49, 63, 77, 91, 105, 119, 133, 147, 161, 175, and 189 and a light chain variable domain comprising an amino acid sequence selected from the group consisting of SEQ ID NOs: 8, 22, 36, 50, 64, 78, 92, 106, 120, 134, 148, 162, 176 and 190 wherein the antibody specifically binds to C3b (e.g., human and/or cynomolgus C3b).

In another aspect, the invention provides (i) an isolated monoclonal antibody having: a full length heavy chain comprising an amino acid sequence that has been optimized for expression in a mammalian cell selected from the group consisting of SEQ ID NOs: 9, 23, 37, 51, 65, 79, 93, 107, 121, 135, 149, 163, 177, and 191, and a full length light chain comprising an amino acid sequence that has been optimized for expression in a mammalian cell selected from the group consisting of SEQ ID NOs: 10, 24, 38, 52, 66, 80, 94, 108, 122, 136, 150, 164, 178, and 192; or (ii) a functional protein comprising an antigen binding portion thereof.

In another aspect, the present invention provides C3b-binding antibodies that comprise the heavy chain and light chain CDR1s, CDR2s and CDR3s as described in Table 1, or combinations thereof. The amino acid sequences of the VH CDR1s of the antibodies are shown in SEQ ID NOs: 1, 15, 29, 43, 57, 71, 85, 99, 113, 127, 141, 155, 169, and 183. The amino acid sequences of the VH CDR2s of the antibodies and are shown in SEQ ID NOs: 2, 16, 30, 44, 58, 72, 86, 100, 114, 128, 142, 156, 170, and 184. The amino acid sequences of the VH CDR3s of the antibodies are shown in SEQ ID NOs: 3, 17, 31, 45, 59, 73, 87, 101, 115, 129, 143, 157, 171, and 185. The amino acid sequences of the VL CDR1s of the antibodies are shown in SEQ ID NOs: 4, 18, 32, 46, 60, 74, 88, 102, 116, 130, 144, 158, 172, and 186. The amino acid sequences of the VL CDR2s of the antibodies are shown in SEQ ID NOs: 5, 19, 33, 47, 61, 75, 89, 103, 117, 131, 145, 159, 173, and 187. The amino acid sequences of the VL CDR3s of the antibodies are shown in SEQ ID NOs: 6, 20, 34, 48, 62, 76, 90, 104, 118, 132, 146, 160, 174, and 188. The CDR regions are delineated using the Kabat system (Kabat, E. A., et al., 1991 Sequences of Proteins of Immunological Interest, Fifth Edition, U.S. Department of Health and Human Services, NIH Publication No. 91-3242).

Given that each of these antibodies can bind to C3b and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched, although each antibody preferably contains a VH CDR1, 2 and 3 and a VL CDR1, 2 and 3 to create other C3b-binding binding molecules of the invention. Such “mixed and matched” C3b-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present invention. In addition to the foregoing, in one embodiment, the antigen binding fragments of the antibodies described herein can comprise a VH CDR1, 2, and 3, or a VL CDR 1, 2, and 3, wherein the fragment binds to C3b as a single variable domain.

Accordingly, the present invention provides an isolated monoclonal antibody or antigen binding region thereof comprising a heavy chain variable region CDR1 comprising an amino acid sequence selected from the group consisting of SEQ ID NOs: 1, 15, 29, 43, 57, 71, 85, 99, 113, 127, 141, 155, 169, and 183; a heavy chain variable region CDR2 comprising an amino acid sequence selected from the group consisting of SEQ ID NOs: 2, 16, 30, 44, 58, 72, 86, 100, 114, 128, 142, 156, 170, and 184; a heavy chain variable region CDR3 comprising an amino acid sequence selected from the group consisting of SEQ ID NOs: 3, 17, 31, 45, 59, 73, 87, 101, 115, 129, 143, 157, 171, and 185; a light chain variable region CDR1 comprising an amino acid sequence selected from the group consisting of SEQ ID NOs: 4, 18, 32, 46, 60, 74, 88, 102, 116, 130, 144, 158, 172, and 186; a light chain variable region CDR2 comprising an amino acid sequence selected from the group consisting of SEQ ID NOs: 5, 19, 33, 47, 61, 75, 89, 103, 117, 131, 145, 159, 173, and 187; and a light chain variable region CDR3 comprising an amino acid sequence selected from the group consisting of SEQ ID NOs: 6, 20, 34, 48, 62, 76, 90, 104, 118, 132, 146, 160, 174, and 188; wherein the antibody specifically binds C3b.

The invention includes an antibody or antigen binding fragment thereof having the heavy and light chain sequences of antibody 9556 in Table 1. The invention includes an antibody or antigen binding fragment thereof having the heavy and light chain sequences of antibody 9611 in Table 1. The invention includes an antibody or antigen binding fragment thereof having the heavy and light chain sequences of antibody 9612 in Table 1. The invention also includes an antibody or antigen binding fragment thereof having the heavy and light chain sequences of antibody 9609 in Table 1. The invention includes an antibody or antigen binding fragment thereof having the heavy and light chain sequences of antibody 9610 in Table 1. The invention further includes an antibody or antigen binding fragment thereof having the heavy and light chain sequences of antibody 9674 in Table 1. The invention still further includes an antibody or antigen binding fragment thereof having the heavy and light chain sequences of antibody 9675 in Table 1. The invention includes an antibody or antigen binding fragment thereof having the heavy and light chain sequences of antibody 9124 in Table 1. The invention also includes an antibody or antigen binding fragment thereof having the heavy and light chain sequences of antibody 9397 in Table 1. The invention also includes an antibody or antigen binding fragment thereof having the heavy and light chain sequences of antibody 9398 in Table 1. The invention further includes an antibody or antigen binding fragment thereof having the heavy and light chain sequences of antibody 9136 in Table 1. The invention also includes an antibody or antigen binding fragment thereof having the heavy and light chain sequences of antibody 9141 in Table 1. The invention still further includes an antibody or antigen binding fragment thereof having the heavy and light chain sequences of antibody 9373 in Table 1. The invention also includes an antibody or antigen binding fragment thereof having the heavy and light chain sequences of antibody 9423 in Table 1.

In a specific embodiment, an antibody that specifically binds to C3b comprising a heavy chain variable region CDR1 of SEQ ID NO:1; a heavy chain variable region CDR2 of SEQ ID NO: 2; a heavy chain variable region CDR3 of SEQ ID NO: 3; a light chain variable region CDR1 of SEQ ID NO: 4; a light chain variable region CDR2 of SEQ ID NO: 5; and a light chain variable region CDR3 of SEQ ID NO: 6. In another specific embodiment, an antibody that specifically binds to C3b comprising a heavy chain variable region CDR1 of SEQ ID NO: 15; a heavy chain variable region CDR2 of SEQ ID NO: 16; a heavy chain variable region CDR3 of SEQ ID NO: 17; a light chain variable region CDR1 of SEQ ID NO: 18; a light chain variable region CDR2 of SEQ ID NO: 19; and a light chain variable region CDR3 of SEQ ID NO: 20.

In another specific embodiment, an antibody that specifically binds to C3b comprising a heavy chain variable region CDR1 of SEQ ID NO: 29; a heavy chain variable region CDR2 of SEQ ID NO: 30; a heavy chain variable region CDR3 of SEQ ID NO: 31; a light chain variable region CDR1 of SEQ ID NO: 32 a light chain variable region CDR2 of SEQ ID NO: 33; and a light chain variable region CDR3 of SEQ ID NO: 34. In another specific embodiment, an antibody that specifically binds to C3b comprising a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 46; a light chain variable region CDR2 of SEQ ID NO: 47; and a light chain variable region CDR3 of SEQ ID NO: 48.

In another specific embodiment, an antibody that specifically binds to C3b comprising a heavy chain variable region CDR1 of SEQ ID NO: 57; a heavy chain variable region CDR2 of SEQ ID NO: 58; a heavy chain variable region CDR3 of SEQ ID NO: 59; a light chain variable region CDR1 of SEQ ID NO: 60; a light chain variable region CDR2 of SEQ ID NO: 61; and a light chain variable region CDR3 of SEQ ID NO: 62. In another specific embodiment, an antibody that specifically binds to C3b comprising a heavy chain variable region CDR1 of SEQ ID NO: 71; a heavy chain variable region CDR2 of SEQ ID NO: 72; a heavy chain variable region CDR3 of SEQ ID NO: 73; a light chain variable region CDR1 of SEQ ID NO: 74; a light chain variable region CDR2 of SEQ ID NO: 75; and a light chain variable region CDR3 of SEQ ID NO: 76.

In another specific embodiment, an antibody that specifically binds to C3b comprising a heavy chain variable region CDR1 of SEQ ID NO: 85; a heavy chain variable region CDR2 of SEQ ID NO: 86; a heavy chain variable region CDR3 of SEQ ID NO: 87; a light chain variable region CDR1 of SEQ ID NO: 88; a light chain variable region CDR2 of SEQ ID NO: 89; and a light chain variable region CDR3 of SEQ ID NO: 90.

In certain embodiments, an antibody that specifically binds to C3b is an antibody that is described in Table 1. In a preferred embodiment, the antibody that binds C3b is antibody 9556. In a further preferred embodiment, the antibody that binds C3b is antibody 9610. In a further preferred embodiment, the antibody that binds C3b is antibody 9674. In a further preferred embodiment, the antibody that binds C3b is antibody 9675. In a still further preferred embodiment, the antibody that binds C3b is antibody 9609.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described below, as well as DP47 and DPK9.

### Homologous Antibodies

In yet another embodiment, the present invention provides an antibody or an antigen-binding fragment thereof comprising amino acid sequences that are homologous to the sequences described in Table 1, and said antibody binds to a C3b protein (e.g., human and/or cynomolgus C3b), and retains the desired functional properties of those antibodies described in Table 1.

For example, the invention provides an isolated monoclonal antibody (or a functional antigen binding fragment thereof) comprising a heavy chain variable domain and a light chain variable domain, wherein the heavy chain variable domain comprises an amino acid sequence that is at least 80%, at least 90%, or at lest 95% identical to an amino acid sequence selected from the group consisting of SEQ ID NOs: 7, 21, 35, 49, 63, 77, 91, 105, 119, 133, 147, 161, 175, and 189; the light chain variable domain comprises an amino acid sequence that is at least 80%, at least 90%, or at least 95% identical to an amino acid sequence selected from the group consisting of SEQ ID NOs: 8, 22, 36, 50, 64, 78, 92, 106, 120, 134, 148, 162, 176 and 190; the antibody specifically binds to C3b (e.g., human and/or cynomolgus C3b), and the antibody can inhibit red blood cell lysis in a hemolytic assay. In a specific example, such antibodies have an IC₅₀ value in a hemolytic assay of less than 50 nM at 10% human or cynomolgus serum.

In other embodiments, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except an amino acid substitution in no more than 1, 2, 3, 4 or 5 amino acid position. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 7, 21, 35, 49, 63, 77, 91, 105, 119, 133, 147, 161, 175, 189, 8, 22, 36, 50, 64, 78, 92, 106, 120, 134, 148, 162, 176 and 190 respectively, followed by testing of the encoded altered antibody for retained function using the functional assays described herein.

In other embodiments, the full length heavy chain and/or full length light chain amino acid sequences may be 50% 60%, 70%, 80%, 90%, 95%, 96%, 97%, 98% or 99% identical to the sequences set forth in Table 1. An antibody having a full length heavy chain and full length light chain having high (i.e., 80% or greater) identity to the full length heavy chains of any of SEQ ID NOs: 9, 23, 37, 51, 65, 79, 93, 107, 121, 135, 149, 163, 177, and 191, and full length light chains of any of SEQ ID NOs 10, 24, 38, 52, 66, 80, 94, 108, 122, 136, 150, 164, 178, and 192, respectively, can be obtained by mutagenesis (e.g., site-directed or PCR-mediated mutagenesis) of nucleic acid molecules encoding such polypeptides respectively, followed by testing of the encoded altered antibody for retained function using the functional assays described herein.

In other embodiments, the full length heavy chain and/or full length light chain nucleotide sequences may be 60%, 70%, 80%, 90%, 95%, 96%, 97%, 98% or 99% identical to the sequences set forth above.

In other embodiments, the variable regions of heavy chain and/or light chain nucleotide sequences may be 60%, 70%, 80%, 90%, 95%, 96%, 97%, 98% or 99% identical to the sequences set forth above.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain embodiments, an antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the C3b-binding antibodies of the invention. Accordingly, the invention provides an isolated monoclonal antibody, or a functional antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 1, 15, 29, 43, 57, 71, 85, 99, 113, 127, 141, 155, 169, and 183, and conservative modifications thereof; the heavy chain variable region CDR2 amino acid sequences are selected from the group consisting of SEQ ID NOs: 2, 16, 30, 44, 58, 72, 86, 100, 114, 128, 142, 156, 170, and 184, and conservative modifications thereof; the heavy chain variable region CDR3 amino acid sequences are selected from the group consisting of SEQ ID NOs: 3, 17, 31, 45, 59, 73, 87, 101, 115, 129, 143, 157, 171, and 185, and conservative modifications thereof; the light chain variable regions CDR1 amino acid sequences are selected from the group consisting of SEQ ID NOs: 4, 18, 32, 46, 60, 74, 88, 102, 116, 130, 144, 158, 172, and 186, and conservative modifications thereof; the light chain variable regions CDR2 amino acid sequences are selected from the group consisting of SEQ ID NOs: 5, 19, 33, 47, 61, 75, 89, 103, 117, 131, 145, 159, 173, and 187, and conservative modifications thereof; the light chain variable regions of CDR3 amino acid sequences are selected from the group consisting of SEQ ID NOs: 6, 20, 34, 48, 62, 76, 90, 104, 118, 132, 146, 160, 174, and 188, and conservative modifications thereof; the antibody or the antigen-binding fragment thereof specifically binds to C3b, and inhibits red blood cell lysis in a hemolytic assay as described herein.

In other embodiments, an antibody of the invention optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the C3b-binding antibodies of the invention. Accordingly, the invention provides an isolated monoclonal antibody optimized for expression in a mammalian cell consisting of a full length heavy chain and a full length light chain wherein: the full length heavy chain has amino acid sequences selected from the group of SEQ ID NOs: 9, 23, 37, 51, 65, 79, 93, 107, 121, 135, 149, 163, 177, and 191, and conservative modifications thereof; and the full length light chain has amino acid sequences selected from the group of SEQ ID NOs: 10, 24, 38, 52, 66, 80, 94, 108, 122, 136, 150, 164, 178, and 192, and conservative modifications thereof; the antibody specifically binds to C3b (e.g., human and/or cynomolgus C3b); and the antibody inhibits red blood cell lysis in a hemolytic assay as described herein. In a specific embodiment, such antibodies have an IC₅₀ value in a hemolytic assay of less than 50 nM at 10% human or cynomolgus serum.

Antibodies that Bind to the Same Epitope

The present invention provides antibodies that bind to the same epitope as do the C3b-binding antibodies described in Table 1. Additional antibodies can therefore be identified based on their ability to cross-compete (e.g., to competitively inhibit the binding of, in a statistically significant manner) with other antibodies of the invention in C3b binding assays. The ability of a test antibody to inhibit the binding of antibodies of the present invention to a C3b protein (e.g., human and/or cynomolgus C3b) demonstrates that the test antibody can compete with that antibody for binding to C3b; such an antibody may, according to non-limiting theory, bind to the same or a related (e.g., a structurally similar or spatially proximal) epitope on the C3b protein as the antibody with which it competes. In a certain embodiment, the antibody that binds to the same epitope on C3b as the antibodies of the present invention is a human monoclonal antibody. Such human monoclonal antibodies can be prepared and isolated as described herein. As used herein, an antibody “competes” for binding when the competing antibody inhibits C3b binding of an antibody of the invention by more than 50%, in the presence of competing antibody concentrations higher than 10⁶×K_{D} of the competing antibody.

### Engineered and Modified Antibodies

An antibody of the invention further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated monoclonal antibody, or an antigen binding fragment thereof, comprising a heavy chain variable region comprising CDR1 sequences having an amino acid sequence selected from the group consisting of SEQ ID NOs: 1, 15, 29, 43, 57, 71, 85, 99, 113, 127, 141, 155, 169, and 183; CDR2 sequences having an amino acid sequence selected from the group consisting of SEQ ID NOs: 2, 16, 30, 44, 58, 72, 86, 100, 114, 128, 142, 156, 170, and 184; CDR3 sequences having an amino acid sequence selected from the group consisting of SEQ ID NOs: 3, 17, 31, 45, 59, 73, 87, 101, 115, 129, 143, 157, 171, and 185, respectively; and a light chain variable region having CDR1 sequences having an amino acid sequence selected from the group consisting of SEQ ID NOs: 4, 18, 32, 46, 60, 74, 88, 102, 116, 130, 144, 158, 172, and 186; CDR2 sequences having an amino acid sequence selected from the group consisting of SEQ ID NOs: 5, 19, 33, 47, 61, 75, 89, 103, 117, 131, 145, 159, 173, and 187; and CDR3 sequences consisting of an amino acid sequence selected from the group consisting of SEQ ID NOs: 6, 20, 34, 48, 62, 76, 90, 104, 118, 132, 146, 160, 174, and 188, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference.

An example of framework sequences for use in the antibodies of the invention are those that are structurally similar to the framework sequences used by selected antibodies of the invention, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, Vl2, and Vk2. Additional frameworks are known in the art and may be found, for example, in the vBase data base on the world wide web at vbase.mrc-cpe.cam.ac.uk/index.php?&MMN_position=1:1.

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Accordingly, in another embodiment, the invention provides isolated C3b-binding monoclonal antibodies, or an antigen binding fragment thereof, consisting of a heavy chain variable region having: a VH CDR1 region consisting of an amino acid sequence selected from the group having SEQ ID NOs: 1, 15, 29, 43, 57, 71, 85, 99, 113, 127, 141, 155, 169, and 183 or an amino acid sequence having one, two, three, four or five amino acid substitutions, deletions or additions as compared to SEQ. ID NOs: 1, 15, 29, 43, 57, 71, 85, 99, 113, 127, 141, 155, 169, and 183; a VH CDR2 region having an amino acid sequence selected from the group consisting of SEQ ID NOs: 2, 16, 30, 44, 58, 72, 86, 100, 114, 128, 142, 156, 170, and 184, or an amino acid sequence having one, two, three, four or five amino acid substitutions, deletions or additions as compared to SEQ ID NOs: 2, 16, 30, 44, 58, 72, 86, 100, 114, 128, 142, 156, 170, and 184; a VH CDR3 region having an amino acid sequence selected from the group consisting of SEQ ID NOs: 3, 17, 31, 45, 59, 73, 87, 101, 115, 129, 143, 157, 171, and 185, or an amino acid sequence having one, two, three, four or five amino acid substitutions, deletions or additions as compared to SEQ ID NOs: 3, 17, 31, 45, 59, 73, 87, 101, 115, 129, 143, 157, 171, and 185; a VL CDR1 region having an amino acid sequence selected from the group consisting of SEQ ID NOs: 4, 18, 32, 46, 60, 74, 88, 102, 116, 130, 144, 158, 172, and 186, or an amino acid sequence having one, two, three, four or five amino acid substitutions, deletions or additions as compared to SEQ ID NOs: 4, 18, 32, 46, 60, 74, 88, 102, 116, 130, 144, 158, 172, and 186; a VL CDR2 region having an amino acid sequence selected from the group consisting of SEQ ID NOs: 5, 19, 33, 47, 61, 75, 89, 103, 117, 131, 145, 159, 173, and 187, or an amino acid sequence having one, two, three, four or five amino acid-substitutions, deletions or additions as compared to SEQ ID NOs: 5, 19, 33, 47, 61, 75, 89, 103, 117, 131, 145, 159, 173, and 187; and a VL CDR3 region having an amino acid sequence selected from the group consisting of SEQ ID NOs: 6, 20, 34, 48, 62, 76, 90, 104, 118, 132, 146, 160, 174, and 188, or an amino acid sequence having one, two, three, four or five amino acid substitutions, deletions or additions as compared to SEQ ID NOs: 6, 20, 34, 48, 62, 76, 90, 104, 118, 132, 146, 160, 174, and 188.

Grafting Antigen-Binding Domains into Alternative Frameworks or Scaffolds

A wide variety of antibody/immunoglobulin frameworks or scaffolds can be employed so long as the resulting polypeptide includes at least one binding region which specifically binds to C3b. Such frameworks or scaffolds include the 5 main idiotypes of human immunoglobulins, or fragments thereof, and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the invention pertains to generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target C3b protein (e.g., human and/or cynomolgus C3b). Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (SciI Proteins GmbH, Halle, Germany).

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

### Human or Humanized Antibodies

The present invention provides fully human antibodies that specifically bind to a C3b protein (e.g., human and/or cynomolgus C3b). Compared to the chimeric or humanized antibodies, the human C3b-binding antibodies of the invention have further reduced antigenicity when administered to human subjects.

The human C3b-binding antibodies can be generated using methods that are known in the art. For example, the humaneering technology used to converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Using one of the mouse or chimeric C3b-binding antibodies described above as the reference antibody, this method can be readily employed to generate human antibodies that bind to human C3b with the same binding specificity and the same or better binding affinity. In addition, such human C3b-binding antibodies can also be commercially obtained from companies which customarily produce human antibodies, e.g., KaloBios, Inc. (Mountain View, Calif.).

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for C3b. In certain embodiments herein, the camelid antibody or nanobody is naturally produced in the camelid animal, i.e., is produced by the camelid following immunization with C3b or a peptide fragment thereof, using techniques described herein for other antibodies. Alternatively, the C3b-binding camelid nanobody is engineered, i.e., produced by selection for example from a library of phage displaying appropriately mutagenized camelid nanobody proteins using panning procedures with C3b as a target as described in the examples herein. Engineered nanobodies can further be customized by genetic engineering to have a half life in a recipient subject of from 45 minutes to two weeks. In a specific embodiment, the camelid antibody or nanobody is obtained by grafting the CDRs sequences of the heavy or light chain of the human antibodies of the invention into nanobody or single domain antibody framework sequences, as described for example in PCT/EP93/02214.

### Bispecific Molecules and Multivalent Antibodies

In another aspect, the present invention features bispecific or multispecific molecules comprising a C3b-binding antibody, or a fragment thereof, of the invention. An antibody of the invention, or antigen-binding regions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for C3b and a second binding specificity for a second target epitope. For example, the second target epitope is another epitope of C3b different from the first target epitope.

Additionally, for the invention in which the bispecific molecule is multi-specific, the molecule can further include a third binding specificity, in addition to the first and second target epitope.

In one embodiment, the bispecific molecules of the invention comprise as a binding specificity at least one antibody, or an antibody fragment thereof, including, e.g., a Fab, Fab′, F(ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bispecific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21). A diabody can be fused to Fc to generate a “di-diabody” (see Lu et al., 2004 J. Biol. Chem., 279(4):2856-65).

Other antibodies which can be employed in the bispecific molecules of the invention are murine, chimeric and humanized monoclonal antibodies.

The bispecific molecules of the present invention can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-l-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)2 or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent compounds comprising at least two identical or different antigen-binding portions of the antibodies of the invention binding to C3b. The antigen-binding portions can be linked together via protein fusion or covalent or non covalent linkage. Alternatively, methods of linkage has been described for the bispecific molecules. Tetravalent compounds can be obtained for example by cross-linking antibodies of the antibodies of the invention with an antibody that binds to the constant regions of the antibodies of the invention, for example the Fc or hinge region.

Trimerizing domain are described for example in Borean patent EP 1 012 280B1. Pentamerizing modules are described for example in PCT/EP97/05897.

Antibodies with Extended Half Life

The present invention provides for antibodies that specifically bind to C3b protein which have an extended half-life in vivo.

Many factors may affect a protein's half life in vivo. For examples, kidney filtration, metabolism in the liver, degradation by proteolytic enzymes (proteases), and immunogenic responses (e.g., protein neutralization by antibodies and uptake by macrophages and dentritic cells). A variety of strategies can be used to extend the half life of the antibodies of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DAR Pins, avimers, affibodies, and anticalins; by genetic fusion to rPEG, albumin, domain of albumin, albumin-binding proteins, and Fc; or by incorporation into nancarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the invention. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defence system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to C3b while a second binding domain of the antibody binds to serum albumin, preferably HSA.

The strategies for increasing half life is especially useful in nanobodies, fibronectin-based binders, and other antibodies or proteins for which increased in vivo half life is desired.

### Antibody Conjugates

The present invention provides antibodies or fragments thereof that specifically bind to a C3b protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a C3b protein may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I,), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and noradioactive paramagnetic metal ions.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alph-emiters such as 213Bi or macrocyclic chelators useful for conjugating radiometal ions, including but not limited to, 131In, 131LU, 131Y, 131Ho, 131Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Methods of Producing Antibodies of the Invention

Nucleic Acids Encoding the Antibodies

The invention provides substantially purified nucleic acid molecules which encode polypeptides comprising segments or domains of the C3b-binding antibody chains described above. Some of the nucleic acids of the invention comprise the nucleotide sequence encoding the heavy chain variable region shown in SEQ ID NO: 7, 21, 35, 49, 63, 77, 91, 105, 119, 133, 147, 161, 175, and 189, and/or the nucleotide sequence encoding the light chain variable region shown in SEQ ID NO: 8, 22, 36, 50, 64, 78, 92, 106, 120, 134, 148, 162, 176. In a specific embodiment, the nucleic acid molecules are those identified in Table 1. Some other nucleic acid molecules of the invention comprise nucleotide sequences that are substantially identical (e.g., at least 65, 80%, 95%, or 99%) to the nucleotide sequences of those identified in Table 1. When expressed from appropriate expression vectors, polypeptides encoded by these polynucleotides are capable of exhibiting C3b antigen binding capacity.

Also provided in the invention are polynucleotides which encode at least one CDR region and usually all three CDR regions from the heavy or light chain of the C3b-binding antibody set forth above. Some other polynucleotides encode all or substantially all of the variable region sequence of the heavy chain and/or the light chain of the C3b-binding antibody set forth above. Because of the degeneracy of the code, a variety of nucleic acid sequences will encode each of the immunoglobulin amino acid sequences.

The nucleic acid molecules of the invention can encode both a variable region and a constant region of the antibody. Some of nucleic acid sequences of the invention comprise nucleotides encoding a mature heavy chain sequence that is substantially identical (e.g., at least 80%, 90%, or 99%) to the mature heavy chain sequence set forth in SEQ ID NO: 9, 23, 37, 51, 65, 79, 93, 107, 121, 135, 149, 163, 177, and 191. Some other nucleic acid sequences comprising nucleotide encoding a mature light chain sequence that is substantially identical (e.g., at least 80%, 90%, or 99%) to the mature light chain sequence set forth in SEQ ID NO: 10, 24, 38, 52, 66, 80, 94, 108, 122, 136, 150, 164, 178, and 192.

The polynucleotide sequences can be produced by de novo solid-phase DNA synthesis or by PCR mutagenesis of an existing sequence (e.g., sequences as described in the Examples below) encoding an C3b-binding antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the C3b-binding antibodies described above. Various expression vectors can be employed to express the polynucleotides encoding the C3b-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the C3b-binding polynucleotides and polypeptides in mammalian (e.g., human) cells include pThioHis A, B & C, pcDNA3.1/H is, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an C3b-binding antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an C3b-binding antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted C3b-binding antibody sequences. More often, the inserted C3b-binding antibody sequences are linked to a signal sequences before inclusion in the vector. Vectors to be used to receive sequences encoding C3b-binding antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the C3b-binding antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express C3b-binding polypeptides of the invention. Insect cells in combination with baculovirus vectors can also be used.

In some preferred embodiments, mammalian host cells are used to express and produce the C3b-binding polypeptides of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express C3b-binding antibody chains or binding fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

Generation of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are human monoclonal antibodies. Such human monoclonal antibodies directed against C3b can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchromosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise C3b-binding antibodies of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise C3b-binding antibodies of the invention. For example, mice carrying both a human heavy chain transchromosome and a human light chain transchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise C3b-binding antibodies of the invention.

Human monoclonal antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Framework or Fc Engineering

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen’. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, LecI3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Methods of Engineering Altered Antibodies

As discussed above, the C3b-binding antibodies having VH and VL sequences or full length heavy and light chain sequences shown herein can be used to create new C3b-binding antibodies by modifying full length heavy chain and/or light chain sequences, VH and/or VL sequences, or the constant region(s) attached thereto. Thus, in another aspect of the invention, the structural features of a C3b-binding antibody of the invention are used to create structurally related C3b-binding antibodies that retain at least one functional property of the antibodies of the invention, such as binding to human C3b and also inhibiting one or more functional properties of C3b (e.g., inhibit red blood cell lysis in a hemolytic assay).

For example, one or more CDR regions of the antibodies of the present invention, or mutations thereof, can be combined recombinantly with known framework regions and/or other CDRs to create additional, recombinantly-engineered, C3b-binding antibodies of the invention, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an C3b-binding antibody consisting of: a heavy chain variable region antibody sequence having a CDR1 sequence selected from the group consisting of SEQ ID NOs: 1, 15, 29, 43, 57, 71, 85, 99, 113, 127, 141, 155, 169, and 183, a CDR2 sequence selected from the group consisting of SEQ ID NOs: 2, 16, 30, 44, 58, 72, 86, 100, 114, 128, 142, 156, 170, and 184, and/or a CDR3 sequence selected from the group consisting of SEQ ID NOs: 3, 17, 31, 45, 59, 73, 87, 101, 115, 129, 143, 157, 171, and 185; and a light chain variable region antibody sequence having a CDR1 sequence selected from the group consisting of SEQ ID NOs: 4, 18, 32, 46, 60, 74, 88, 102, 116, 130, 144, 158, 172, and 186, a CDR2 sequence selected from the group consisting of SEQ ID NOs: 5, 19, 33, 47, 61, 75, 89, 103, 117, 131, 145, 159, 173, and 187, and/or a CDR3 sequence selected from the group consisting of SEQ ID NOs: 6, 20, 34, 48, 62, 76, 90, 104, 118, 132, 146, 160, 174, and 188; altering at least one amino acid residue within the heavy chain variable region antibody sequence and/or the light chain variable region antibody sequence to create at least one altered antibody sequence; and expressing the altered antibody sequence as a protein.

Accordingly, in another embodiment, the invention provides a method for preparing an C3b-binding antibody optimized for expression in a mammalian cell consisting of: a full length heavy chain antibody sequence having a sequence selected from the group of SEQ ID NOs: 9, 23, 37, 51, 65, 79, 93, 107, 121, 135, 149, 163, 177, and 191; and a full length light chain antibody sequence having a sequence selected from the group of SEQ ID NOs: 10, 24, 38, 52, 66, 80, 94, 108, 122, 136, 150, 164, 178, and 192; altering at least one amino acid residue within the full length heavy chain antibody sequence and/or the full length light chain antibody sequence to create at least one altered antibody sequence; and expressing the altered antibody sequence as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence(s) is one that retains one, some or all of the functional properties of the C3b-binding antibodies described herein, which functional properties include, but are not limited to, specifically binding to human and/or cynomolgus C3b; and the antibody inhibit red blood cell lysis in a hemolytic assay.

The functional properties of the altered antibodies can be assessed using standard assays available in the art and/or described herein, such as those set forth in the Examples (e.g., ELISAs).

In certain embodiments of the methods of engineering antibodies of the invention, mutations can be introduced randomly or selectively along all or part of an C3b-binding antibody coding sequence and the resulting modified C3b-binding antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Characterization of the Antibodies of the Invention

The antibodies of the invention can be characterized by various functional assays. For example, they can be characterized by their ability to inhibit red blood cell lysis in hemolytic assays, their affinity to a C3b protein (e.g., human and/or cynomolgus C3b), their ability to inhibit C3a or C5a generation, their ability to inhibit C3b deposition, the epitope binning, their resistance to proteolysis, and their ability to block the complement cascade, for example, their ability to inhibit MAC formation.

Various methods can be used to measure presence of complement pathway molecules and activation of the complement system (see, e.g., U.S. Pat. No. 6,087,120; and Newell et al., J Lab Clin Med, 100:437-44, 1982). For example, the complement activity can be monitored by (i) measurement of inhibition of complement-mediated lysis of red blood cells (hemolysis); (ii) measurement of ability to inhibit cleavage of C3 or C5; and (iii) inhibition of alternative pathway mediated hemolysis.

The two most commonly used techniques are hemolytic assays (see, e.g., Baatrup et al., Ann Rheum Dis, 51:892-7, 1992) and immunological assays (see, e.g., Auda et al., Rheumatol Int, 10:185-9, 1990). The hemolytic techniques measure the functional capacity of the entire sequence-either the classical or alternative pathway. Immunological techniques measure the protein concentration of a specific complement component or split product. Other assays that can be employed to detect complement activation or measure activities of complement components in the methods of the present invention include, e.g., T cell proliferation assay (Chain et al., J Immunol Methods, 99:221-8, 1987), and delayed type hypersensitivity (DTH) assay (Forstrom et al., 1983, Nature 303:627-629; Halliday et al., 1982, in Assessment of Immune Status by the Leukocyte Adherence Inhibition Test, Academic, New York pp. 1-26; Koppi et al., 1982, Cell. Immunol. 66:394-406; and U.S. Pat. No. 5,843,449).

In hemolytic techniques, all of the complement components must be present and functional. Therefore hemolytic techniques can screen both functional integrity and deficiencies of the complement system (see, e.g., Dijk et al., J Immunol Methods 36: 29-39, 1980; Minh et al., Clin Lab Haematol. 5:23-34 1983; and Tanaka et al., J Immunol 86: 161-170, 1986). To measure the functional capacity of the classical pathway, sheep red blood cells coated with hemolysin (rabbit IgG to sheep red blood cells) or chicken red blood cells that are sensitized with rabbit anti-chicken antibodies are used as target cells (sensitized cells). These Ag-Ab complexes activate the classical pathway and result in lysis of the target cells when the components are functional and present in adequate concentration. To determine the functional capacity of the alternative pathway, rabbit red blood cells are used as the target cell (see, e.g., U.S. Pat. No. 6,087,120).

To test the ability of an antibody to inhibit MAC (membrane attack complex) formation, a MAC deposition assay can be performed. Briefly, zymosan can be used to activate the alternative pathway and IgM can be used to active the classic pathway. Fabs are pre-incubated with human serum and added to plates coated with zymosan or IgM. Percentage inhibition of MAC deposition can be calculated for each sample relative to baseline (EDTA treated human serum) and positive control (human serum).

To test the ability of an antibody of the invention to inhibit complement protein C3 in the alternative pathway is to measure the generation of the C3 breakdown product C3b depositing on zymosan. Specific methods for measuring C3b deposition are described in detail in the Examples below.

The ability of an antibody to inhibit generation of the C5 breakdown product C5a can be measured by, for example, ELISA assay using a specific anti-05a antibody, such as the mouse anti-human C5a-des-Arg antibody available from US Biologics.

The ability of an antibody to bind to C3b can be detected by labelling the antibody of interest directly, or the antibody may be unlabelled and binding detected indirectly using various sandwich assay formats known in the art.

In some embodiments, the C3b-binding antibodies of the invention block or compete with binding of a reference C3b-binding antibody to a C3b polypeptide. These can be fully human C3b-binding antibodies described above. They can also be other mouse, chimeric or humanized C3b-binding antibodies which bind to the same epitope as the reference antibody. The capacity to block or compete with the reference antibody binding indicates that a C3b-binding antibody under test binds to the same or similar epitope as that defined by the reference antibody, or to an epitope which is sufficiently proximal to the epitope bound by the reference C3b-binding antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as a C3b polypeptide. A test antibody competes with the reference antibody for specific binding to the antigen if an excess of the test antibody substantially inhibits binding of the reference antibody. Substantial inhibition means that the test antibody reduces specific binding of the reference antibody usually by at least 10%, 25%, 50%, 75% or 90%.

There are a number of known competition binding assays that can be used to assess competition of a C3b-binding antibody with the reference C3b-binding antibody for binding to a C3b protein. These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using 1-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified antigen bound to a solid surface or cells bearing either of these, an unlabelled test C3b-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected C3b-binding monoclonal antibodies bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using a C3b polypeptide coated-ELISA plates. Biotinylated MAb binding can be detected with a strep-avidin-alkaline phosphatase probe. To determine the isotype of a purified C3b-binding antibody, isotype ELISAs can be performed. For example, wells of microtiter plates can be coated with 1 μg/ml of anti-human IgG overnight at 4° C. After blocking with 1% BSA, the plates are reacted with 1 μg/ml or less of the monoclonal C3b-binding antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined.

To demonstrate binding of monoclonal C3b-binding antibodies to live cells expressing a C3b polypeptide, flow cytometry can be used. Briefly, cell lines expressing C3b (grown under standard growth conditions) can be mixed with various concentrations of a C3b-binding antibody in PBS containing 0.1% BSA and 10% fetal calf serum, and incubated at 37° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

C3b-binding antibodies of the invention can be further tested for reactivity with a C3b polypeptide or antigenic fragment by Western blotting. Briefly, purified C3b polypeptides or fusion proteins, or cell extracts from cells expressing C3b can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St: Louis, Mo.).

Examples of functional assays are also described in the Example section below.

### Prophylactic and Therapeutic Uses

The present invention provides methods of treating a disease or disorder associated with increased complement activity by administering to a subject in need thereof an effective amount of the antibodies of the invention. In a specific embodiment, the present invention provides a method of treating age-related macular degeneration (AMD) by administering to a subject in need thereof an effective amount of the antibodies of the invention.

The antibodies of the invention can be used, inter alia, to prevent progression of dry AMD to wet AMD, to slow and/or prevent progression of geographic atrophy, to treat or prevent macular edema, and to improve vision lost due to dry AMD progression. It can also be used in combination with anti-VEGF therapies for the treatment of wet AMD patients.

In some embodiments, the present invention provides methods of treating a complement related disease or disorder by administering to a subject in need thereof an effective amount of the antibodies of the invention. Examples of known complement related diseases or disorders include: neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, myocardial infarction, balloon angioplasty, post-pump syndrome in cardiopulmonary bypass or renal bypass, hemodialysis, renal ischemia, mesenteric artery reperfusion after acrotic reconstruction, infectious disease or sepsis, immune complex disorders and autoimmune diseases, rheumatoid arthritis, systemic lupus erythematosus (SLE), SLE nephritis, proliferative nephritis, hemolytic anemia, and myasthenia gravis. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, and immune complex-associated inflammation.

In a specific embodiment, the present invention provides methods of treating a complement related disease or disorder by administering to a subject in need thereof an effective amount of the antibodies of the invention, wherein said disease or disorder is asthma, arthritis (e.g., rheumatoid arthritis), autoimmune heart disease, multiple sclerosis, inflammatory bowel disease, ischemia-reperfusion injuries, Barraquer-Simons Syndrome, hemodialysis, systemic lupus, lupus erythematosus, psoriasis, multiple sclerosis, transplantation, diseases of the central nervous system such as Alzheimer's disease and other neurodegenerative conditions, aHUS, glomerulonephritis, bullous pemphigoid or MPGN II.

In a specific embodiment, the present invention provides methods of treating glomerulonephritis by administering to a subject in need thereof an effective amount of a composition comprising an antibody of the present invention. Symptoms of glomerulonephritis include, but not limited to, proteinuria; reduced glomerular filtration rate (GFR); serum electrolyte changes including azotemia (uremia, excessive blood urea nitrogen—BUN) and salt retention, leading to water retention resulting in hypertension and edema; hematuria and abnormal urinary sediments including red cell casts; hypoalbuminemia; hyperlipidemia; and lipiduria. In a specific embodiment, the present invention provides methods of treating paroxysmal nocturnal hemoglobinuria (PNH) by administering to a subject in need thereof an effective amount of a composition comprising an antibody of the present invention.

In a specific embodiment, the present invention provides methods of reducing the dysfunction of the immune and hemostatic systems associated with extracorporeal circulation by administering to a subject in need thereof an effective amount of a composition comprising an antibody of the present invention. The antibodies of the present invention can be used in any procedure which involves circulating the patient's blood from a blood vessel of the patient, through a conduit, and back to a blood vessel of the patient, the conduit having a luminal surface comprising a material capable of causing at least one of complement activation, platelet activation, leukocyte activation, or platelet-leukocyte adhesion. Such procedures include, but are not limited to, all forms of ECC, as well as procedures involving the introduction of an artificial or foreign organ, tissue, or vessel into the blood circuit of a patient.

Subjects to be treated with therapeutic agents of the present invention can also be administered other therapeutic agents with know methods of treating conditions associated with macular degeneration, such as antibiotic treatments as described in U.S. Pat. No. 6,218,368. In other treatments, immunosuppressive agents such as cyclosporine, are agents capable of suppressing immune responses. These agents include cytotoxic drugs, corticosteriods, nonsteroidal anti-inflammatory drugs (NSAIDs), specific T-lymphocyte immunosuppressants, and antibodies or fragments thereof (see Physicians' Desk Reference, 53rd edition, Medical Economics Company Inc., Montvale, N.J. (1999). Immunosuppressive treatment is typically continued at intervals for a period of a week, a month, three months, six months or a year. In some patients, treatment is administered for up to the rest of a patient's life.

When the therapeutic agents of the present invention are administered together with another agent, the two can be administered sequentially in either order or simultaneously. In some aspects, an antibody of the present invention is administered to a subject who is also receiving therapy with a second agent (e.g., verteporfin). In other aspects, the binding molecule is administered in conjunction with surgical treatments.

Suitable agents for combination treatment with C3b-binding antibodies include agents known in the art that are able to modulate the activities of complement components (see, e.g., U.S. Pat. No. 5,808,109). Other agents have been reported to diminish complement-mediated activity. Such agents include: amino acids (Takada, Y. et al. Immunology 1978, 34, 509); phosphonate esters (Becker, L. Biochem. Biophy. Acta 1967, 147, 289); polyanionic substances (Conrow, R. B. et al. J. Med. Chem. 1980, 23, 242); sulfonyl fluorides (Hansch, C.; Yoshimoto, M. J. Med. Chem. 1974, 17, 1160, and references cited therein); polynucleotides (DeClercq, P. F. et al. Biochem. Biophys. Res. Commun. 1975, 67, 255); pimaric acids (Glovsky, M. M. et al. J. Immunol. 1969, 102, 1); porphines (Lapidus, M. and Tomasco, J. Immunopharmacol. 1981, 3, 137); several antiinflammatories (Burge, J. J. et al. J. Immunol. 1978, 120, 1625); phenols (Muller-Eberhard, H. J. 1978, in Molecular Basis of Biological Degradative Processes, Berlin, R. D. et al., eds. Academic Press, New York, p. 65); and benzamidines (Vogt, W. et al Immunology 1979, 36, 138). Some of these agents function by general inhibition of proteases and esterases. Others are not specific to any particular intermediate step in the complement pathway, but, rather, inhibit more than one step of complement activation. Examples of the latter compounds include the benzamidines, which block C1, C4 and C3b utilization (see, e.g., Vogt et al. Immunol. 1979, 36, 138).

Additional agents known in the art that can inhibit activity of complement components include K-76, a fungal metabolite from Stachybotrys (Corey et al., J. Amer. Chem. Soc. 104: 5551, 1982). Both K-76 and K-76 COOH have been shown to inhibit complement mainly at the C3b step (Hong et al., J. Immunol. 122: 2418, 1979; Miyazaki et al., Microbiol. Immunol. 24: 1091, 1980), and to prevent the generation of a chemotactic factor from normal human complement (Bumpers et al., Lab. Clinc. Med. 102: 421, 1983). At high concentrations of K-76 or K-76 COOH, some inhibition of the reactions of C2, C3, C6, C7, and C9 with their respective preceding intermediaries is exhibited. K-76 or K-76 COOH has also been reported to inhibit the C3b inactivator system of complement (Hong et al., J. Immunol. 127: 104-108, 1981). Other suitable agents for practicing methods of the present invention include griseofulvin (Weinberg, in Principles of Medicinal Chemistry, 2d Ed., Foye, W. O., ed., Lea & Febiger, Philadelphia, Pa., p. 813, 1981), isopannarin (Djura et al., Aust. J. Chem. 36: 1057, 1983), and metabolites of Siphonodictyon coralli-phagum (Sullivan et al., Tetrahedron 37: 979, 1981).

A combination therapy regimen may be additive, or it may produce synergistic results (e.g., reductions in complement pathway activity more than expected for the combined use of the two agents). In some embodiments, the present invention provide a combination therapy for preventing and/or treating AMD or another complement related disease as described above with a C3b-binding antibody of the invention and an anti-angiogenic, such as anti-VEGF agent.

### Diagnostic Uses

In one aspect, the invention encompasses diagnostic assays for determining C3b protein and/or nucleic acid expression as well as C3b protein function, in the context of a biological sample (e.g., blood, serum, cells, tissue) or from individual is afflicted with a disease or disorder, or is at risk of developing a disorder associated with AMD.

Diagnostic assays, such as competitive assays rely on the ability of a labelled analogue (the “tracer”) to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results in order to quantitatively determine the amount of analyte present in the test sample. These assays are called ELISA systems when enzymes are used as the detectable markers. In an assay of this form, competitive binding between antibodies and C3b-binding antibodies results in the bound C3b protein, preferably the C3b epitopes of the invention, being a measure of antibodies in the serum sample, most particularly, neutralising antibodies in the serum sample.

A significant advantage of the assay is that measurement is made of neutralising antibodies directly (i.e., those which interfere with binding of C3b protein, specifically, epitopes). Such an assay, particularly in the form of an ELISA test has considerable applications in the clinical environment and in routine blood screening.

Immunologic techniques employ polyclonal or monoclonal antibodies against the different epitopes of the various complement components (e.g., C3, C4, C5) to detect, e.g., the split products of complement components (see, e.g., Hugli et al., Immunoassays Clinical Laboratory Techniques 443-460, 1980; Gorski et al., J Immunol Meth 47: 61-73, 1981; Linder et al., J Immunol Meth 47: 49-59, 1981; and Burger et al., Immunol 141: 553-558, 1988). Binding of the antibody with the split product in competition with a known concentration of labeled split product could then be measured. Various assays such as radio-immunoassays, ELISA's, and radial diffusion assays are available to detect complement split products.

The immunologic techniques provide high sensitivity to detect complement activation, since they allow measurement of split-product formation in blood from a test subject and control subjects with or without macular degeneration-related disorders. Accordingly, in some methods of the present invention, diagnosis of a disorder associated with ocular disorders is obtained by measurement of abnormal complement activation through quantification of the soluble split products of complement components in blood plasma from a test subject. The measurements can be performed as described, e.g., in Chenoweth et al., N Engl J Med 304: 497-502, 1981; and Bhakdi et al., Biochim Biophys Acta 737: 343-372, 1983. Preferably, only the complement activation formed in vivo is measured. This can be accomplished by collecting a biological sample from the subject (e.g., serum) in medium containing inhibitors of the complement system, and subsequently measuring complement activation (e.g., quantification of the split products) in the sample.

In the clinical diagnosis or monitoring of patients with disorders associated with ocular diseases or disorders, the detection of complement proteins in comparison to the levels in a corresponding biological sample from a normal subject is indicative of a patient with disorders associated with macular degeneration.

In vivo diagnostic or imaging is described in US2006/0067935. Briefly, these methods generally comprise administering or introducing to a patient a diagnostically effective amount of a C3b binding molecule that is operatively attached to a marker or label that is detectable by non-invasive methods. The antibody-marker conjugate is allowed sufficient time to localize and bind to complement proteins within the eye. The patient is then exposed to a detection device to identify the detectable marker, thus forming an image of the location of the C3b binding molecules in the eye of a patient. The presence of C3b binding antibody or an antigen-binding fragment thereof is detected by determining whether an antibody-marker binds to a component of the eye. Detection of an increased level in selected complement proteins or a combination of protein in comparison to a normal individual without AMD disease is indicative of a predisposition for and/or on set of disorders associated with macular degeneration. These aspects of the invention are also preferred for use in eye imaging methods and combined angiogenic diagnostic and treatment methods.

The invention also pertains to the field of predictive medicine in which diagnostic assays; prognostic assays, pharmacogenomics, and monitoring clinical trials are used for prognostic (predictive) purposes to thereby treat an individual prophylactically.

The invention also provides for prognostic (or predictive) assays for determining whether an individual is at risk of developing a disorder associated with dysregulation of complement pathway activity. For example, mutations in a C3b gene can be assayed in a biological sample. Such assays can be used for prognostic or predictive purpose to thereby prophylactically treat an individual prior to the onset of a disorder characterized by or associated with C3b protein, nucleic acid expression or activity.

Another aspect of the invention provides methods for determining C3b nucleic acid expression or C3b protein activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs) on the expression or activity of C3b protein in clinical trials.

### Pharmaceutical Compositions

The invention provides pharmaceutical compositions comprising the C3b-binding antibodies (intact or binding fragments) formulated together with a pharmaceutically acceptable carrier. The compositions can additionally contain one or more other therapeutic agents that are suitable for treating or preventing a complement-associated disease (e.g., AMD). Pharmaceutically acceptable carriers enhance or stabilize the composition, or can be used to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be intravenous, intramuscular, intraperitoneal, or subcutaneous, or administered proximal to the site of the target. In a specific embodiment, the antibodies of the invention are formulated so that they can be administered intravitreally into the eye. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the C3b-binding antibody is employed in the pharmaceutical compositions of the invention. The C3b-binding antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies of the invention employed in the pharmaceutical composition at levels lower than that required to achieve the desired therapeutic effect and gradually increase the dosage until the desired effect is achieved. In general, effective doses of the compositions of the present invention, for the treatment of an allergic inflammatory disorder described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. For systemic administration with an antibody, the dosage ranges from about 0.0001 to 100 mg/kg, and more usually 0.01 to 15 mg/kg, of the host body weight. An exemplary treatment regime entails systemic administration once per every two weeks or once a month or once every 3 to 6 months. For intravitreal administration with an antibody, the dosage ranges from about 0.0001 to about 10 mg. An exemplary treatment regime entails systemic administration once per every two weeks or once a month or once every 3 to 6 months.

Antibody is usually administered on multiple occasions. Intervals between single dosages can be weekly, monthly or yearly. Intervals can also be irregular as indicated by measuring blood levels of C3b-binding antibody in the patient. In some methods of systemic administration, dosage is adjusted to achieve a plasma antibody concentration of 1-1000 μg/ml and in some methods 25-500 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The following examples are provided to further illustrate the invention but not to limit its scope. Other variants of the invention will be readily apparent to one of ordinary skill in the art and are encompassed by the appended claims.

### Example 1

### Production of Antigens and Quality Control

### Generation of Biotinylated C3b

Purified C3b was biotinylated using labeling reagents from Pierce, at a 20-fold molar excess of biotinylation reagent. Biotinylation was performed at room temperature, and unconjugated biotin was separated using 0.5 ml Zeba Spin Desalting Columns. Lysine residues of C3b were labeled using EZ-Link NHS-LC-LC-Biotin, and cysteine residue was labeled using EZ-Link Maleimide-PEG2-Biotin. The degree of biotinylation was quantified using the HABA Assay and LC-MS/MS. Biotinylation of the single cysteine that is involved in thioester bond formation on C3 was confirmed by LC-MS/MS.

### Generation of C3b Bound to Agarose Beads

Purified C3b (Quidel A413, lot 903726) was buffer exchanged into Coupling Buffer (50 mM Tris, 5 mM EDTA-Na, pH 8.5) using PD-10 Desalting columns from Amersham Biosciences (17-0851-01). The SulfoLink Coupling Gel (Pierce 20401) and all other reagents were equilibrated to room temperature. SulfoLink Coupling Gel was equilibrated with 4 gel-bed volume of Coupling Buffer and spun down and supernatant removed. Then the buffer-exchanged C3b protein solution was added to spun down equilibrated SulfoLink Coupling Gel. The mixture was rocked for 15 minutes at room temperature and then left to sit for 30 minutes without mixing. Then the conjugated C3b-coupling gel was washed with 3 gel-bed volumes of Coupling Buffer. Afterwards, one gel-bed volume of Quenching Reagent (50 mM L-Cyteine-HCL (44889) in coupling buffer) was added to the C3b-coupling gel and left to rock for 15 minutes at room temperature. After the 15 minutes, the conjugated C3b-coupling gel was left at room temperature without mixing for 30 minutes. The conjugated C3b-coupling gel was washed with at least 6 gel-bed volumes of wash solution (1 M NaCl) and then washed with 2 gel-bed volumes of degassed Storage Buffer (Phosphate-buffered saline containing 0.05% sodium azide). The final step is to add one gel-bed volume of Storage Buffer to an estimated 1 mg/ml of protein to gel-bed volume.

C3b-SulfoLink Coupling Gel Protein Concentration Determination

The following amounts of C3b were run on a 4-12% denaturing protein gel under reduced conditions: 2 μg, 1.5 μg, 1 μg, 0.75 μg, 0.5 μg, and 0.25 μg. Next to these lanes, 2 μl, 4 μl, and 8 μl of 50% bead suspension (C3b-Coupling Gel) were also loaded at reducing conditions. Since the alpha chain is covalently linked to the beads, it will not appear on the protein gel, thus protein concentration is determined by comparing the beta chain. It was estimated that there was about 1 μg of C3b to 1 μl of coupling gel thus achieving greater than 90% coupling efficiency.

### Calculating Percent Active C3b on SulfoLink Coupling Gel

Four controls set up with Factor B at 4 different concentrations (0.122 μM 0.244 μM, 0.367 μM and 0.489 μM), fixed concentration of soluble C3b (0.294 μM for all 4), and fixed concentration of Factor D (0.47 μM for all 4). Factor P is omitted from these control reactions. The reactions were incubated at 37° C. for 15 minutes. At this point all the control samples were immediately added to 4× sample buffer and put at 95 C for 10 minutes to run later on a 4-12% Bis-Tris Protein Gel. The C3b-coupled beads were at a concentration of about 1 mg C3b per 1 mg bed volume. The following 7 reactions consisted of fixed concentration of coupled C3b beads at 0.294 μM, fixed Factor P at 2.68 μM, and fixed Factor D at 0.95 μM. The concentrations for Factor B were as follows: 0.367 μM, 0.489 μM, 0.978 μM, 1.467 μM, 1.955 μM, 2.933 μM, and 3.910 μM. For these 7 reactions, everything but Factor D was incubated at 37° C. for 30 minutes. After this 30 incubation, 0.95 uM Factor D was added and the reaction incubated for 2 minutes. At this point all the samples were immediately added to 4× sample buffer and run on a 4-12% Bis-Tris gel under reducing conditions. When analyzing data, the Bb band is compared in all lanes.

### Bead-C3b Stability

Post C3b-SulfoLink Coupling gel conjugation, the beads were spun down and re-suspended gently in 100% glycerol (making a final glycerol concentration of 50%). The beads were then put at −80° C. for several freeze thaws (up to 3 tested). Beads were then thawed on ice and transferred to a 15 ml conical tube. 5 column volumes of 1×PBS was added to resuspend the beads. This was spun down at 850 g for 5 min. 2 additional washes with 10 column volumes of 1×PBS was completed. The final step was to resuspend the beads with 1×PBS for a final 50% slurry solution. Each freeze thaw was tested for Bb generation: incubation of 3 μM Factor B, 0.5 μM Factor D, 1 μM of C3b-Beads, and 5 mM MgCl for 1 hour and look for complete Bb generation. In addition to this, frozen beads were tested after storage at −80 C for several weeks via Bb generation.

### Purification of Cyno C3 and Generation of Cyno C3b

Cyno plasma was purchased from Alphagenesis (Yemassee, S.C.). 50 ml plasma was diluted to 200 ml by PBS, 10 mM EDTA and 2 complete cocktail inhibitor tablets (Roche). 40% PEG6000 was added to the solution slowly to a final concentration of 4% and stirred gently at 4 degree for additional 30 min. The precipitation was removed by centrifugation at 17,500 rpm for 20 min. PEG6000 was again added to the supernatant to a final concentration of 12.5% and stirred at 4 degree for 30 min. The supernatant was discarded after centrifugation at 175,000 rpm for 20 min. The pellet was re-dissolved in 50 ml 1×PBS, 10 mM EDTA buffer and the C3 containing solution was passed twice to a 15 ml Protein G (GE) column to remove cyno IgGs. The flow through from protein G column was dialyzed against 4 L 20 mM Tris pH 8.0, 10 mM EDTA for overnight. Meanwhile, the 20 ml MonoQ column (GE) was cleared by 0.5M NaOH, followed by water and large volume of 20 mM Tris pH8.0 and 10 mM EDTA until the column baseline was clear and equilibrated. The dialyzed solution was then loaded to the MonoQ column by ATKA 100 (GE) with a flow rate of 0.8 ml/min. After loading, the column was washed by 10 column volume of 20 mM Tris pH 8.0 and 10 mM EDTA or until the baseline reached to be stable. The protein was eluted off the column by 20 column volume of NaCl linear gradient from 0 to 500 mM and fractions were collected at 4 ml/tube. C3 protein peak was identified by SDS-PAGE gels for fractions under unreduced and reduced condition. C3 was further confirmed by western blot and MS peptide mapping analysis. The 85% pure C3 fractions were then pooled and further purified by 2660 sephacryl 300 gel filtration column (GE) using PBS buffer. The C3 peak fractions were again verified by SDS-PAGE and MS analysis. The pure fractions were pooled and concentrated by millipore concentrator to about 1 mg/ml, aliquoted and stored at −80 degree freezer for later use.

Cyno C3 was diluted to 500 ug/ml in PBS buffer. The C3 was completely converted to C3b by adding 0.4 μM fB (Comptech), 0.05 μM fD (CompTech) and 5 mM MgCl₂ in PBS buffer and incubated at room temperature for 30 min. The C3b was then further purified by 2660 Sephacryl 300 gel filtration column. The C3b containing peak fractions were pooled at concentrated by millipore concentrator. The activity of cyno C3b was tested in C3 convertase assay. The protein showed comparable Bb generation activity to human C3b (CompTech).

### Quality Control of C3b Reagents by Binding by Complement Factors and Commercial Antibodies

Enzyme Linked Immunosorbent Assay (ELISA) Binding (Epitope Conservation)

Biotinylated C3b molecules were compared to non-biotinylated C3b in an ELISA to assess conservation of C3b epitopes recognized by commercially available antibodies.

A Maxisorp plate was coated with 100 μl/well commercially available anti-C3 or anti-C3b antibodies at 2 μg/ml in coating buffer (bicarbonate pH 9.5-9.8) and was incubated overnight at 4° C. After washing 3× with PBST, the plate was blocked with 300 μl/well diluent (Synblock, AbD Serotec) for 2 h at room temperature. After aspirating the blocking solution, 100 μl C3b (+/−biotin) samples diluted in diluent were incubated for 1 h at room temperature. 100 μl/well Strep-HRP (poly-HRP streptavidin) diluted 1:5000 in diluent (poly-HRP diluent) or HRP-conjugated anti-C3 Ab was added for 30 min. After washing 4× with PBST, 100 μl/well TMB Substrate (Ultra TMB substrate solution) was added for 5-10 min. Reaction was stopped with 50 μl/well stop solution (2N H₂SO₄). Absorbance was read (A450-A570) and data were analyzed using SoftMax Pro.

Binding by Complement Factors and Commercial Abs

C3b immobilized on agarose beads was tested for its ability to bind to commercial antibodies and complement factor proteins.

4 μl of C3b bead slurry (corresponding to ˜1 μg of C3b) was added to each tube. The beads were resuspended into 100 μl diluent. The total volume in the tube was then brought up to 200 μl with diluent+Ab or complement factor protein. The tubes were rocked at room temperature for 1 h, then washed once with diluent. Bead slurry was the applied to a mini column, and washed 2× more. Quickly span the rest of the liquid out (1,200 G for 1 min). Plugged the columns, then added 500 μl of secondary antibody in diluent (1:5000), or anti-complement factor Ab. Incubated for 1 h at room temperature, then washed 4× with diluent. [For complement factor, repeated step above with secondary Ab]. Quickly span the rest of the liquid out (1,200 G for 1 min). Plugged the columns, then added 100 μl of TMB substrate. Quickly span the liquid out (1,200 G for 1 min) into a fresh tube. Transferred the solution to 96-well plate. Reaction was stopped with 50 μl/well stop solution (2N H₂SO₄). Absorbance was read (A450-A570) and data were analyzed using SoftMax Pro.

### Example 2

### Generation of C3b-Specific Antibodies from the HuCAL GOLD® Library

Anti-C3b antibodies were generated by selection of clones having high binding affinities using as the source of antibody variant proteins, a commercially available phage display library, the Morphosys HuCAL GOLD® Library. The HuCAL GOLD® Library is a Fab library (Knappik et al., 2000) in which all six CDRs are diversified by appropriate mutation, and which employs the CysDisplay™M technology for linking the Fab to the phage surface (see, e.g., WO01/05950).

HuCAL GOLD® phage-antibodies are provided as 12 separate sublibraries: VH1κ, VH1λ, VH2κ, VH2λ, VH3κ, VH3λ, VH4κ, VH4λ, VH5κ, VH5λ, VH6κ, VH6λ. The 12 sublibraries can be pooled in any combination according to the requirements of the specific experiment. For selection of antibodies binding to C3b, three different panning strategies were applied:
a) solution pannings with biotinylated human C3b where the phage-antigen complex was captured by Streptavidin magnetic beads,
b) a bead based panning, where C3b was bound to agarose beads and
c) differential peptide pannings, where selection rounds on peptides coupled to carrier protein were alternated with selection rounds on full-length C3b (either biotinylated of bound to agarose beads).

### Phagemid Rescue, Phage Amplification and Purification

The HuCAL GOLD® library was amplified in 2×YT medium containing 34 μg/ml chloramphenicol and 1% glucose (2×YT-CG). After infection with VCSM13 helper phage at an OD₆₀₀ₙₘ of 0.5 (30 min at 37° C. without shaking; 30 min at 37° C. shaking at 250 rpm), cells were spun down (4120 g; 5 min; 4° C.), resuspended in 2×YT/34 μg/ml chloramphenicol/50 μg/ml kanamycin/0.25 mM IPTG and grown overnight at 22° C. Phage were PEG-precipitated from the supernatant, resuspended in PBS/20% glycerol and stored at −80° C. Phage amplification between two panning rounds was conducted as follows: mid-log phase E. coli TG1 cells were infected with eluted phage and plated onto LB-agar supplemented with 1% of glucose and 34 μg/ml of chloramphenicol (LB-CG plates). After overnight incubation at 30° C., the TG1 colonies were scraped off the agar plates and used to inoculate 2×YT-CG until an OD₆₀₀ₙₘ of 0.5 was reached. VCSM13 helper phage were added for infection as described above.

### Solution Panning

The antigen used in this panning strategy is biotinylated C3b. Two different biotinylated variants of biotinylated C3b exist. In one variant the Biotin is linked to C3b via a maleimide-PEG-linker attached to a cysteine residue on C3b. This variant is called C3b-cysteine-biotin below. In the other variant the Biotin is linked to C3b via a sulfo-NHS-LCLC-linker attached to 3 different lysine residues on C3b. This variant is called. C3b-lysine-biotin below. Selections on the two different variants are alternated in selection rounds in order not to select phage binding to the linker molecules.

Streptavidin magnetic beads (Dynabeads M-280; Dynal) were washed once with PBS and blocked with Chemiblocker for 2 h at RT. The PBS diluted phage were blocked also with Chemiblocker for 1-2 h at RT on a rotator. The blocked phage were twice pre-adsorbed against blocked Streptavidin magnetic beads for 30 min. The phage supernatant was transferred to a new blocked 2 ml reaction tube and human biotinylated C3b was added and the mixture was incubated for 1-2 h at RT on a rotator. 100 μl of the blocked Streptavidin magnetic beads were added to each panning pool an incubated for 20 min on a rotator. The beads were collected with a particle separator (Dynal MPC-E) for approx. 2.5 min and the solution was removed carefully.

Beads were then washed 7× in PBST using a rotator, followed by washing another three times with PBS. Elution of phage from the Dynabeads was performed by adding 200 μl of 20 mM DTT in 10 mM Tris/HCl pH 8 to each tube and incubation for 10 min. Dynabeads were removed by the magnetic particle separator and the supernatant was added to 14 ml of an E. coli TG-1 culture grown to OD₆₀₀ₙₘ of 0.6-0.8. For phage infection, the culture was incubated in 50 ml plastic tubes for 45 min at 37° C. without shaking. After centrifugation for 5 min at 4120×g, the bacterial pellets were resuspended each in 800 μl 2×YT medium, plated on 3×YT-CG agar plates and incubated overnight at 37° C. Colonies were scraped off the plates and phage were rescued and amplified as described herein. The second and third rounds of selection were performed in an identical way to the first round of selection.

In order to select C3b-specific phage, several different blocking approaches with C3 protein were applied in various subpools. Generally purified C3 or serum containing C3 was added when phage were blocked with Chemiblocker and incubated for 1-2 h on a rotator. Thus potential C3b/C3 crossreactive phage should already be bound to antigen when coming in contact with C3b and only C3b-specific phage should be selected. For panning 1812.1 purified C3 was added at a molar excess of 10-fold (during the 1^{st} round only). No blocking was applied for subpools 1-6 of panning 1889. For subpools 7-12 various blocking conditions using human serum was applied in all three rounds of selection. For subpools 7 and 8 undiluted human serum was added, resulting in a molar excess of C3 over C3b of approximately 70-fold. Because we were worried that serum factors might degrade C3b, a proteinase inhibitor was added (Pefabloc SC, Roche, final concentration 4 mM). For subpools 9-12 diluted human serum was added resulting in a molar excess of C3 over C3b of approximately 2-fold. Subpools 9 and 10 contained proteinase inhibitor, for pools 11 and 12 no inhibitor was added.

### Bead Based Panning

The antigen used in the bead based panning was C3b coupled to sulfolink agarose beads. The beads used for pre-adsorption of phage to blocked beads was produced by MorphoSys, by treating agarose beads (SulfoLink Coupling Gel) with cysteine (from SulfoLink Immobilization Trial Kit), which blocks all the possible binding sites on the beads.

The PBS diluted phage were blocked with Chemiblocker for 1-2 h at RT on a rotator. The blocked phage were twice pre-adsorbed against blocked agarose beads for 30 min. The phage supernatant was transferred to a new blocked 2 ml reaction tube and agarose beads coupled to human C3b were added and the mixture was incubated for 1-2 h at RT on a rotator. The agarose beads were harvested by centrifugation in a tabletop centrifuge (1000 g, 1 min) and the supernatant was discarded. The pellet was washed repeatedly by resuspending gently in 1 ml of washing solution, incubation in washing buffer and harvesting by centrifugation.

Elution of phage from the agarose beads was performed by adding 200 μl of 20 mM DTT in 10 mM Tris/HCl pH 8 to each tube and incubation for 10 min. The beads were pelleted by centrifugation and the supernatant was added to 14 ml of an E. coli TG-1 culture grown to OD₆₀₀ₙₘ of 0.6-0.8. For phage infection, the culture was incubated in 50 ml plastic tubes for 45 min at 37° C. without shaking. After centrifugation for 5 min at 4120×g, the bacterial pellets were resuspended each in 800 μl 2×YT medium, plated on 3×YT-CG agar plates and incubated overnight at 37° C. Colonies were scraped off the plates and phage were rescued and amplified as described herein. The second and third rounds of selection were performed in an identical way to the first round of selection.

The pellet of agarose beads is difficult to see and dissolves easily when the supernatant. Therefore it was decided to use at least 25 μl of the beads in order to be able to see the pellet. This results in a relatively high antigen concentration, which for the 1^{st} round differs for all subpools, since different volumes were used. The concentration of C3b in the subpools of the 1^{st} round was approximately as follows: 189 nM in subpool 1820.1, 128 nM in subpool 1820.2, 257 nM in subpool 1820.3, 98 nM in subpool 1820.4, 66 nM in subpool 1820.5 and 1820.6. In the 2^{nd} round of selection the C3b concentration was 112 nM for subpools 1820.1-3 and 57 nM for subpool 1820.4-6. In the 3^{rd} round of selections the concentration of C3b was 57 nM in all subpools.

Blocking with C3 was applied in the first round for subpools 1820.4-6 by adding purified C3 to a final concentration of approximately 475 nM.

### Differential Peptide Panning

The antigens used in the differential peptide pannings were peptides representing different epitopes on C3b. These peptides had been identified as C3b-specific in a protein structure analysis comparing the surface exposed residues on C3b versus C3. Coupling to the two different carrier proteins, BSA and Transferrin, was performed by MorphoSys as described above. The two different carrier proteins have to be alternated during selection rounds in order not to select phage binding to the carrier protein. In addition rounds of selections on peptides were alternated with selection rounds on full length C3b to ensure binding of the selected phage to the correctly folded full length C3b.

The actual panning procedure in a peptide panning is a solid phase panning using the peptide coupled carrier protein as an antigen bound to Maxisorp plates. A suitable number of wells (depending on the volume of the pre-blocked phage) of a Maxisorp plate (F96 Nunc-Immunoplate) were coated with 300 μl of the carrier protein coupled to the peptides at a concentration of 50 μg/ml in PBS. The plate was sealed and incubated overnight at 4° C.

The coated wells were washed 2× with 400 μl PBS and blocked with 350 μl PBS/5% milk powder for 2 h at RT on a microtiter plate shaker. The phage were blocked with PBST/5% milk powder and the uncoupled carrier protein at a final concentration of 0.5% (v/v) for 2 h at room temperature on a rotator. The coated wells were washed 2× with 400 μl PBS after the blocking procedure. 300 μl of pre-blocked phage were added to each coated well and incubated for 2 h at RT on a shaker. Washing was performed by adding several times 400 μl PBST, followed by washing several times with PBS (see tables 8 and 10 for details).

Elution of phage from the plate was performed with 300 μl 20 mM DTT in 10 mM Tris/HCl pH8 per well for 10 min. The DTT phage eluate was added to 14 ml of E. coli TG1, which were grown to an OD₆₀₀ of 0.6-0.8 at 37° C. in 2YT medium and incubated in 50 ml plastic tubes for 45 min at 37° C. without shaking for phage infection. After centrifugation for 5 min at 4120×g, the bacterial pellets were each resuspended in 600 μl 2×YT medium, plated on 3×YT-CG agar plates and incubated overnight at 37° C. Colonies were scraped off the plates and phage were rescued and amplified as described herein.

For panning 1849 the 1^{st} and 3^{rd} rounds of selection were peptide panning performed as described above. The 2^{nd} round was a selection on C3b bound to agarose beads as described herein with slight variations: binding sites on the agarose beads used for phage pre-adsorption had been blocked using milk powder instead of cystein, and also the phage were preblocked in PBST/5% milk powder as described above. For panning 1883 the 1^{st} and 3^{rd} rounds of selection were solution pannings using biotinylated C3b performed as described herein. The 2^{nd} round was a peptide panning as described above (this section).

### Panning Results

Clones selected after three rounds of panning had been subcloned into the expression vector and then screened for binding to either C3b or the peptide used in the selections. Clones showing binding signals at least 2-fold over background level were considered primary hits.

The output of the first solution panning 1812 was screened for binding to biotinylated C3b on Neutravidin plates yielding 531 primary hits. A screen on biotinylated C3 performed in parallel with the C3b-screen identified 78 clones, which showed stronger signals on C3b than on C3. Sequence analysis of the 78 clones revealed 27 unique sequences, 19 of which could be consolidated and purified. Only a small proportion of these clones showed cross-reactivity to cyno C3b in capture ELISA. Cyno cross reactive C3bneo antibodies were identified as desirable trait of the antibodies of the invention, and C3bneo binders were specifically selected for cyno cross reactivity. Cyno C3b protein was purified from Cyno monkey plasma and the pure cyno C3b protein was used as antigen along with human C3b during the screen process. Cyno monkey is excellent non-human primate safety/tox species. The potency and affinity of C3bneo antibodies to cyno were desired to be within 5-10× of human. This criterion was selected in order to achieve pronounced inhibition of C3b concentrations in the cyno and hence allow us to evaluate the potential toxicities caused by pronounced inhibition of C3b concentrations. At screening phase, many clones had to be discarded due to no or weak cyno cross reactivity. In order to identify more cyno cross reactive clones, a re-screening of panning 1812 was performed. For the re-screen 178 clones were chosen, which had shown significant binding to C3b on Neutravidin plates (at least 5-fold over background) and had not been sequenced before. 38 out of 178 showed binding to cyno C3b and were taken further into the C3 counter-screen. 10 out of 38 clones passed the counter-screen, resulting in 1 new unique clone, which was purified.

An ELISA using sulfhydryl-plates was employed for screening of the bead based panning 1820. Out of 79 primary hits 49 showed stronger signals on C3b than on C3. These were sequenced resulting in 13 unique sequences, 7 of which could be consolidated and purified.

Microexpressed Fabs derived from differential peptide panning 1849 were screened for binding to the peptide used in the respective selection. Carrier proteins coupled to the peptides were used as directly coated antigens in an ELISA. 566 primary hits out of 2944 were identified, but only 22 of the primary hits showed signals at least 5-fold over background. These 22 clones and 32 additional ones, which showed signals close to 5-fold over background were taken further into a screen to check for binding to full length C3b. None of the 54 clones showed binding to full length C3b.

In contrast to the previous differential peptide panning, two rounds of selection on the full length C3b and only one round on the peptide was performed in the differential peptide panning 1883. The output of panning 1883 was screened for binding to C3b in a capture ELISA. The screening of 4416 clones yielded 497 primary hits. The 275 primary hits, which had shown binding signals at least 5-fold over background, were taken further into the counter screen. 183 clones which passed the counter screen were sequenced resulting in 9 unique clones. 7 of the unique clones could be consolidated and were purified.

The output of the second solution panning 1889 was screened for binding to C3b in capture ELISA, yielding 2878 out of 4416 primary hits. Most of the primary hits ( 2469/2878) showed binding signals of at least 5-fold over background. It was decided to take 396 representative clones forward into the counter-screen derived from different panning subpools and having with different signal strengths. 158 out of 396 clones passed the counter-screen and were sequenced, resulting in 14 unique sequences and finally 11 purified Fabs. In order not to loose any interesting clones, the remaining clones with binding signals of 5-fold over background to C3b, which had not been taken into the counter screen (2073/2469) were tested for binding to cyno C3b. The resulting 129 cyno cross-reactive clones were screened for C3b vs. C3 specificity. 25 clones showed C3b selectivity over C3 binding, finally resulting in 4 new unique sequences. 3 out of the 4 clones could be consolidated and purified.

### Epitope Bining

Purified Fabs were biotinylated using a Kit (ECL Protein biotinylation Module, GE) according to the maufacture's instructions. The biotinylated Fab was cleaned from unbound biotin by running it over a Zeba Desalt Spin Column (Pierce). The binding activity to human C3b of the biotinylated Fab was tested in direct comparison to its unbiotinylated progenitor in a capture ELISA (described herein). Only Fabs whose binding activity was not influenced by biotinylation were taken forward.

Epitope binnings were performed by competition ELISA. The capture antibody used is an antibody directed to the C3d protein rabbit polyclonal anti human C3d Ab, Abcam), which is a sub-domain of C3b. Wells of a Maxisorp plate were filled with the capture antibody diluted to 2 μg/ml in PBS. The plate was sealed and incubated overnight at 4° C.

On the next day remaining binding sites on the plate were blocked by adding PBST/5% milk powder. The plate was incubated for 1 h at RT and then washed 2× with PBST. C3b was added at a final concentration of 2.5 μg/ml diluted in PBST/0.5% milk powder. The plate was incubated for 1 h at RT and then washed 2× with PBST.

At the same time C3b at a final concentration of 2.5 μg/ml, biotinylated Fab and unbiotinylated Fab (100-fold molar excess over biotinylated Fab), all diluted in PBS were added to the wells. The plate was incubated for 1 h at RT and then washed 2× with PBST.

For detection of biotinylated Fabs AP-conjugated Streptavidin (Zymed) was added, the plate was incubated for 1 h at RT and then washed 5× with TBST. The fluorogenic substrate AttoPhos was used according to manufacturer's instructions. Fluorescence was measured in a Tecan GENios Pro plate reader.

Epitope binnings were performed by competition ELISA as described above. A mixture of biotinylated Fab and unbiotinylated Fab (100-fold excess) was added to human C3b. the biotin label was detected. The signal obtained for negative control Fab MOR03207 was set as the 100% values. Inhibition was ranked in relation to the 100% signal.

Six epitope groups were identified (summarized in the table below). Groups B and C and also Groups D and E share partly overlapping epitopes.

TABLE 2
Summary of Epitope Groups

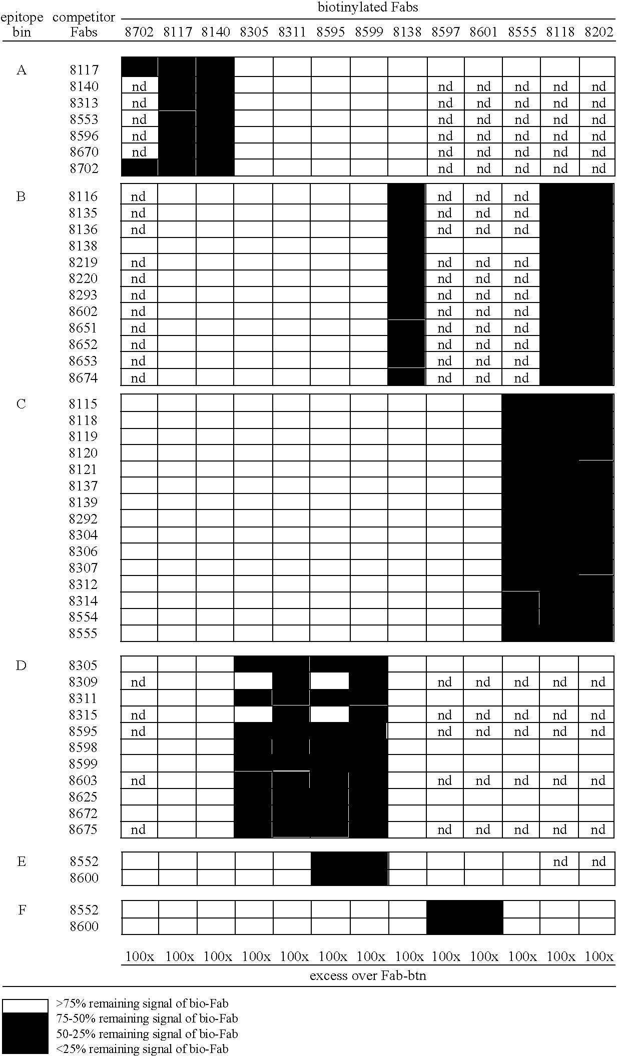

### Example 3

### Affinity Maturation and Optimization

### Generation of Affinity Maturation Libraries

To increase affinity and biological activity of selected antibody fragments, L-CDR3 and H-CDR2 regions were optimized in parallel by cassette mutagenesis using trinucleotide directed mutagenesis (Virnekas et al., 1994), while the framework regions were kept constant. Prior to cloning for affinity maturation, all parental Fab fragments were transferred from the corresponding expression vector (pMORPH®X9_FH) into the CysDisplay™ vector pMORPH®25_LHC via XbaI/EcoRI. pMORPH®25_LHC was created from the HuCAL GOLD® display vector pMORPH®23_LHC by removal of one BssHII site interfering with library cloning for H-CDR2 optimization.

For optimizing L-CDR3 of parental Fabs, the L-CDR3, framework 4 and the constant region of the light chains (405 bp) were removed by BpiI/SphI and replaced by a repertoire of diversified L-CDR3s together with framework 4 and the constant domain. Approximately 1.5 μg of the Fab vector fragment were ligated with a 3-5 fold molar excess of the insert fragment carrying the diversified L-CDR3s. In a second library set the H-CDR2 (XhoI/BssHII) was diversified, while the connecting framework regions were kept constant. In order to monitor the cloning efficiency the parental H-CDR2 was replaced by a dummy, before the diversified H-CDR2 cassette was cloned in.

Ligation mixtures of the libraries were electroporated in 4 ml E. coli TOP10F cells (Invitrogen, Carlsbad, Calif., USA) yielding from 10⁸ to 10⁹ independent colonies. This library size ensured coverage of the theoretical diversity. Amplification of the library was performed as described herein. For quality control single clones were randomly picked and sequenced.

Preparation of Phage for Affinity Maturation Pannings

The HuCAL® maturation libraries were amplified in 2×YT medium containing 34 μg/ml chloramphenicol and 1% glucose (2×YT-CG). After infection with VCSM13 helper phage at an OD₆₀₀ₙₘ of 0.5 (30 min at 37° C. without shaking; 30 min at 37° C. shaking at 250 rpm), cells were harvested (4120×g; 5 min; 4° C.), resuspended in 2×YT/34 μg/ml chloramphenicol/50 μg/ml kanamycin/0.25 mM IPTG and grown o/n at 22° C. Phage were PEG-precipitated twice from the supernatant, resuspended in PBS and used for the maturation pannings described below.

### (a) Maturation Panning

The selection procedure employed in maturation was a solution panning as described above. In order to increase panning stringency and to select for improved off-rates, antigen concentration was decreased and prolonged washing conditions (up to 24 h) were applied. The overnight washing step was performed at 4° C., all other washing steps were performed at RT.

### Selection of Candidates for Affinity Maturation

The Fabs derived from primary panning had been characterized in various assays. They were ranked and grouped as potential maturation candidates according to the following criteria:
Selectivity of binding to C3b versus binding to C3
Potency in hemolytic assay
Potency in C3b and MAC Deposition assays
Mode of action
Epitope bin
Affinity

8 primary antibodies were taken into maturation, which are therefore called “parental” antibodies below.

The parental antibodies MOR08035, 8598 and 8599 belong to the biggest group of Fabs found after primary panning. They are members of epitope bin D, display activity in functional assays, and inhibit C3- and C5-convertase. The binding of factor B to C3b is strongly inhibited by these antibodies, the binding of, factor H to a lesser extend.

Parental antibody MOR08552 displays the same characteristics, except it has a slightly different epitope (bin C).

MOR08672 and MOR08675 both belong to epitope bin D. They inhibit binding of factor H quite well, but show only weak inhibition of the binding of other factors. They might act via a different mechanism than the antibodies above. Also the lack of competition by other factors could result in the same potency being achieved with a lower affinity. This argument might especially apply to MOR08675 which shows reasonable potency although its affinity to C3b is really low.

MOR08555 is a member of epitope group C, has features similar characteristics as the MOR08305-group, but does not inhibit the C5-convertase.

MOR08653 belongs to epitope bin B, which partially overlaps with bin C. Its presence in functional assays results in inhibition of all mechanisms tested: binding of factor P, factor B and factor H, inhibition of C3b-dimer formation and inhibition of C3- and C5-convertase.

The maturation libraries from each parental were constructed separately in order to be able to maintain a flexibility in the composition of the panning pools. At a later timepoint the pools were compiled. For each parental antibody a target K_{D} value was defined according to the main mechanism of inhibition exerted by that antibody. MOR08598 and MOR08653 and also MOR08555 and MOR08599 were compiled in pools of 2 each. Within each pool the antibodies display similar affinities to the antigens, are well expressed, have the same target K_{D} and do not share overlapping epitopes.

### Libraries for Affinity Maturation

Affinity maturation was performed by parallel exchange of LCDR3 and HCDR2 cassettes. The CDR sequences were optimized by trinucleotide-directed cassette mutagenesis. Fab fragments from expression vector pMORPH®x9_Fab_MH were cloned into the phagemid vector pMORPH®25.

16 different affinity maturation libraries (one LCDR3 and one HCDR3 library for each parental antibody) were generated by standard cloning procedures and transformation of the diversified clones into electro-competent E. coli TOP10F′ cells (Invitrogen). Library sizes were good, being in the range of 5×10⁸-4×10⁹. Sequencing of randomly picked clones showed a diversity of 100%. No parental binders were found among the picked clones. Finally phage of all 16 libraries were prepared separately.

### Panning Strategies for Affinity Maturation

HCDR2 and LCDR3 libraries were kept separately during selection. 4 of the 8 parentals were treated as leads; the other 4 parentals were arranged in pools of 2 each. About 10¹² phage rescued from the generated affinity maturation libraries were subjected to maturation pannings.

Solution pannings using the respective phage pools were performed using biotinylated antigen, alternating between human and cyno C3b. In order to increase panning stringency and to select for improved off-rates, antigen concentration was decreased and prolonged washing conditions (up to 24 h) were applied. Panning and washing conditions are summarized above. After maturation pannings, the enriched phagemid pools were sub-cloned into pMORPH®x9_MH expression vector.

### Affinity Screening and Maturation Pannings Outcome

A total of 2464 clones derived from all pannings were screened as bacterial lysates for improved affinities on human C3b. Preliminary affinities were estimated by solution equilibrium titration (SET). Clones estimated to be approved in affinity compared to their parental clone were considered primary hits.

Primary hits were obtained from each of the panning subpools and all primary hits were sequenced, except for the 8305-LCDR3 library, where the best 17 out of 65 primary hits were sequenced. In total 173 unique sequences were identified out of 261 primary hits sequenced. Derivatives from all parental Fabs except for MOR08598 could be identified. No HCDR2-matured derivatives were identified from parentals MOR08552 and MOR08599.

### Selection of Affinity Optimized Fabs for Protein Purification

Unique primary hits were picked into microwell culture plates and used for microexpression of Fab. The Fab lysate was used in secondary screening testing for binding to human C3b, cyno C3b in capture ELISA, checking cross-reactivity to counter targets C3d ad C5 in capture ELISA and performing the counterscreen. Clones passing the screens were taken forward. The aim was to express and purify approximately 12 derivatives from each parental in large scale. In case that more than 12 derivatives passed the secondary screening, the selection for protein purification was based on sequence variability. The following paragraphs describe the selection process for each panning subpool in detail.

From parental MOR08305 23 unique clones matured in HCDR2 and 11 clones matured in LCDR3 had been identified. 21/23 HCDR2-matured clones passed the counter-screen and 18/21 bound well to human C3b. But only 1/18 clones showed good binding to cyno C3b. This clone (MOR09124) was chosen for large scale purification. Additional 5 clones were chosen for large scale purification according to sequence variability. Purification did not work for MOR09122. 3/11 of the LCDR3-matured clones did not pass the counter-screen, but ⅔ were included in large scale purification (MOR09130 and 9131), because their high binding signals on human C3b implied a low affinity which in turn would lead to residual binding on serum. Purification did not work for MOR09132.

From parental 8552 16 LCDR3-matured derivatives had been identified. 7/16 of the derivatives were chosen for large scale purification according to similar criteria as described above. But purification failed for all of them. Therefore all remaining 9 derivatives were taken into large scale purification. The purification worked only for 2/9 (MOR09308 and 9313).

From parental MOR08555 1 unique clone matured in HCDR2 and 3 clones matured in LCDR3 had been identified. Since there were only 4 derivatives in total, it was decided to purify all of them, irrespective of their behaviour in secondary screening.

From parental MOR08599 25 unique clones matured in LCDR3 had been identified. Most of them performed well in all tests performed during secondary screening. The selection of clones for large scale purification was based on sequence variability.

From parental MOR08653 25 unique clones matured in HCDR2 and 10 unique clones matured in LCDR3 had been identified. Only 1 out of each subset performed well in the counter-screen. These clones (MOR09198 and 9202) were taken forward. The selection of the other clones was based on sequence variability.

From parental MOR08672 5 unique clones matured in HCDR2 and 29 unique clones matured in LCDR3 had been identified. Only ⅕ HCDR2-matured clones passed the counter-screen and was taken forward (MOR09139). Two additional HCDR2-matured clones were selected. The purification only worked for MOR09137. Most of the LCDR3-matured clones performed well in secondary screening. The selection of these clones was based on sequence variability.

From parental MOR08675 7 unique clones matured in HCDR2 and 17 unique clones matured in LCDR3 had been identified. 1/7 HCDR2-matured showed only weak binding to human and cyno C3b and was excluded. Out of the remaining 6 clones only 3 did not contain a potential glycosylation site and were taken into large scale purification. 2 additional clones containing a potential glycosylation site were included in the selection. Most of the LCDR3-matured clones performed well in secondary screening. The selection of these clones was based on sequence variability.

### Selection of Affinity Optimized Fabs to be Taken Further

After reviewing all the available data presented in the previous sections, including data relating to the characterization of the affinity optimized Fabs (e.g., binding affinity, inhibition of hemolysis, inhibition of C3b deposition) which are not shown, the best Fabs were selected to be taken further. The selection consists of 22 matured Fabs, which are derived from 4 different parental antibodies. Table 5 summarizes the key characteristics of the selected Fabs.

TABLE 5
Key characteristics of 22 matured Fabs selected to be taken further

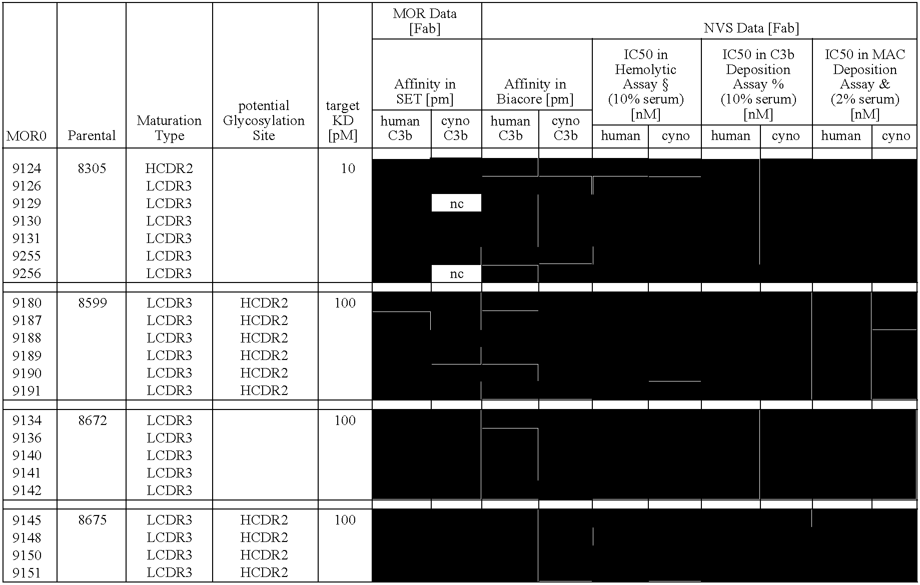

nc not calcuated due to incomplete titration
§ MUST criterion for IgG IC50 ≦ 5 nM for human serum and IC50 ≦ 150 nM for cyno serum
% MUST criterion for IgG IC50 ≦ 20 nM for human serum and IC50 ≦ 60 nM for cyno serum
& MUST criterion for IgG IC50 ≦ 5 nM for human serum and IC50 ≦ 15 nM for cyno serum

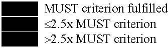

### IgG Conversion of Optimized Fabs and Cross-Cloning on IgG Level

All 8 parental Fabs, several matured Fabs and several matured repaired (potential glycosylation sites removed) Fabs with desired profile were subcloned into a human IgG format.

Cross-cloning on IgG level is achieved by transfecting cells with combinations of light and heavy chain constructs. Since MOR09124 was the only HCDR2 matured clone identified, cross-cloning was only possible for the respective family (MOR08305-derivatives), where the heavy chain of MOR09124 was combined with the light chain of the other matured family members. The cross-clones were given new MOR numbers, which are summarized in table 8.

TABLE 8
MOR numbers of cross-cloned antibodies.

| | | Heavy Chain | Light Chain |
|---|---|---|---|
| MOR0 | Type | from MOR0 | from MOR0 |
| 9395 | cross-clone | 9124 | 9128 |
| 9396 | cross-clone | 9124 | 9129 |
| 9397 | cross-clone | 9124 | 9130 |
| 9398 | cross-clone | 9124 | 9131 |
| 9399 | cross-clone | 9124 | 9255 |
| 9400 | cross-clone | 9124 | 9256 |

Of the foregoing affinity matured Fabs, repaired Fabs (glycosylation sites removed), and cross-cloned Fabs, the binding characteristics of Fabs 9124, 9397, 9398, 9136, 9141, 9373, and 9423 were determined according to the methods described herein. Table 9 summarizes the binding affinity, functional potency, and inhibition of complement factor binding for these Fabs.

TABLE 9

| | Affinity | | | | Functional Potency, IC50 | | | | | |
|---|---|---|---|---|---|---|---|---|---|---|
| | KD (pM), | | KD (pM), | | HA (nM), | | C3b Dep | | MAC Dep | |
| | Biacore | | SET | | 10% | | (nM), | | (nM), | |
| Fab | huC3b | cynoC3b | huC3b | cyno C3b | human | cyno | human | cyno | human | cyno |
| 9124 | 7 | 33 | 1 | 21 | 44 | 101 | 16 | 73 | 1 | 5 |
| 9397 | 7 | 89 | 2 | 120 | 35 | 59 | 13 | 35 | 1 | 7 |
| 9398 | 3 | 92 | 4 | 100 | 45 | 86 | 13 | 56 | 1 | 5 |
| 9136 | 75 | 5 | 96 | 16 | 32 | 52 | 8 | 3 | 2 | 3 |
| 9141 | 39 | 4 | 100 | 20 | 27 | 43 | 7 | 9 | 1 | 2 |
| 9373 | 53 | 9 | 33 | 78 | 41 | 62 | 24 | 5 | 1 | 6 |
| 9423 | 75 | 486 | 100 | 340 | 48 | 83 | 14 | 66 | 1 | 4 |

| | Inhibition of | | | | | | | |
|---|---|---|---|---|---|---|---|---|
| | factors binding | | | | | | | |
| | MOA, IC50 (nM) | | | Cross reactivity | | | | |
| Fab | C3b-fB | C3b-fP | C3b-fH | C5 | C3d | iC3b | C3(H2O) | C3c |
| 9124 | 3 | No | 1 | No | No | Yes | Yes | Yes |
| 9397 | 0.1 | 300 | 0.1 | No | No | Yes | Yes | Yes |
| 9398 | 0.1 | 300 | 0.1 | No | No | Yes | Yes | Yes |
| 9136 | No | No | No | No | No | Yes | Yes | Yes |
| 9141 | No | No | No | No | No | Yes | Yes | Yes |
| 9373 | 0.3 | No | 0.1 | No | No | Yes | Yes | Yes |
| 9423 | 0.2 | No | 0.08 | No | No | Yes | Yes | Yes |

The methods used to determine the binding affinities and functional properties of the Fabs summarized in Table 8 are described in detail below. As can be seen from the data in Table 8, the Fab fragments of the invention are able to bind both human and cynomolgus C3b with an affinity of less than or equal to 100 pM, and in many cases less than or equal to 10 pM. In addition, the Fab fragments demonstrate functional potency against both human and cynomolgus C3b with an IC50 of less than or equal to 100 nM, and in most cases less than or equal to 50 nM.

### Example 4

### Generation and Characterization of IgG Format C3b Binders

### Germlining of IgGs

Regions in pM2 expression vectors coding for the immunoglobulin variable regions of light and heavy chain were germlined and optimized by Geneart (Geneart AG, Regensburg, Germany) in order to match the germlined sequence, to avoid codons which are unsuitable for expression in mammalian cells and to avoid cryptic splice sites. The N-terminal QVQ of all heavy chains was changed to EVQ, as a terminal Q might form pyroglutamine. Antibodies from each parental family were chosen for germlining/optimization. Briefly, antibodies were germlined and expressed in an IgG format as follows.

### Conversion to IgG

In order to express full length immunoglobulin, variable domain fragments of Fab heavy (VH) and light chains (VL) were subcloned from the Fab expression vectors into IgG1 expression vectors. Restriction enzymes MfeI, and BlpI were used for subcloning of the VH domain fragment into pMORPH®2_h_IgG1AA, in which leucines at positions 234 and 235 were mutated to alanines to abrogate FcRγ binding and attenuate effector functions. Subcloning of the VL domain fragment into pMORPH®2_h_Igκ was performed via the EcoRV and BslWI sites, whereas subcloning into pMORPH®2_h_Igλ2 was done using EcoRV and HpaI.

### Transient Expression and Purification of Human IgG

Eukaryotic HKB11 and HEK293 cells were transfected with an 1:1 ratio of IgG heavy and light chain expression vector DNA. Cell culture supernatant was harvested at 3 or 7 days post transfection and subjected to standard protein A affinity chromatography (MabSelect SURE, GE Healthcare). As not otherwise stated, buffer exchange was performed to 1× Dulbcecco's PBS (pH 7.2, Invitrogen) and samples were sterile filtered (0.2 μm). Purity of IgG was analyzed under denaturing, reducing and non-reducing conditions in SDS-PAGE or by using Agilent BioAnalyzer and in native state by HP-SEC.

Germlined antibodies were given new MOR numbers, as shown in table 10.

TABLE 10
MOR numbers of germlined antibodies.

| MOR0 of Germlined Antibody | MOR0 of Progenitor Antibody |
|---|---|
| 9555 | 9140 |
| 9556 | 9124 |
| 9609 | 9136 |
| 9610 | 9141 |
| 9611 | 9397 |
| 9612 | 9398 |
| 9613 | 9314 |
| 9674 | 9373 |
| 9675 | 9423 |

Of these, germlined antibodies 9556, 9611, 9612, 9609, 9610, 9674, and 9675 were characterized further.

### Affinity Determination

C3bneo antibodies block complement activation by binding to neoepitopes on C3b molecule. The neoepitopes on C3b are also binding sites for other abundant complement proteins in the plasma, e.g., factor H, factor B, and factor P, which regulate complement activation through alternative pathway. C3bneo antibodies, therefore, should have high affinity in order to compete with these abundant complement proteins to effectively block the complement activation by blocking these complement protein binding to C3b. The high affinity is required to achieve low therapeutic dose. For example, concentration of factor H in plasma is around 3 μM and binding affinity (Kd) of factor H to C3b is ˜30 nM; factor H concentration is 100 fold higher than its Kd. When C3bneo antibodies compete with factor H for binding to C3b, it will require 1 nM concentration of 10 pM Kd C3bneo antibody (100× antibody concentration above its Kd) to inhibit 50% of factor H binding to C3b. Equal or less than 10 pM Kd C3bneo antibody, therefore, will achieve effective blockade of alternative pathway complement activation with appropriate therapeutic antibody dose. Accordingly, antibody molecules of the invention are selected to have high binding affinity in the range of less than or equal to 65 pM, preferably less than or equal to 10 pM. For example, antibody molecules of the invention are selected to have a binding affinity for C3b of 9, 8, 7, 6, 5, 4, 3, or 2 pM or less (i.e., a higher affinity of less than 2 pM).

The affinity of the germlined IgG antibodies for binding C3b was determined by Biacore and solution equilibrium titration (SET) as follows.

Biacore Determination

Biacore kinetics experiments were done with the BIAcore T100 (GE Healthcare) using CM5 sensor chips (GE Healthcare, BR-1005-30) at 25° C. The running buffer was HBS-EP(+) (GE Healthcare, BR-1001-88). Briefly, the following steps were carried out to determine binding affinity.
Prepare anti-C3d IgG immobilized sensor chip: Rabbit anti-C3d polyclonal antibody (Abcam, ab-15981) (50 ug/ml in acetate pH5.0 coupling buffer (GE Healthcare, BR-1003-51)) was coupled to two different flow cells (Fc1 and 2) on a CM5 chip at 10 ul/min flow rate for 600 seconds by using amino-coupling procedure according to the supplier's instruction (GE Healthcare, BR-1000-50). The final immobilized level will be >7000 RU.
Capture C3b on second flow cell: 1 ug/ml of C3b in running buffer was injected at 10 ul/min on second flow cell (Fc2) to reach capture level ˜70 RU for Fab or ˜20 RU for IgG kinetics analysis.
Inject anti-C3b Fab or IgG at different concentration on both flow cells: Inject anti-C3b solution (0.3125 nM˜10 nM in running buffer; at 1:2 serial dilutions) on both flow cells (Fc1 and 2) at 60 ul/min for 240 seconds.
Dissociation: Inject HBS-EP(+) running buffer at 60 ul/min on both flow cells to monitor the dissociation between C3b and anti-C3b Fab/IgG. Dissociation time was set at 2400 seconds for 5 nM and 2.5M Fab/IgG concentrations and at 300 seconds for all other concentrations including another 5 nM Fab/IgG concentration.
Regeneration: Regeneration was performed at the end of each cycle on both flow cells with Glycine-HCl pH1.6 (made from Glycine-HCl pH1.5 and Glycine pH2.0, GE Healthcare)+0.05% P20 surfactant (GE Healthcare, BR-1000-54) at a flow rate of 60 ul/min for 40 seconds twice.
Kinetics analysis: Kinetic rate constants was obtained by applying 1:1 binding model with BIAevaluation 1.1 software, wherein the Rmax values were fit locally.

The results of the Biacore binding kinetics determination are shown in Table 11 below. As shown the antibodies described herein exhibit high affinity binding to human C3b, with KD values typically less than or equal to 10 pM, and in many cases less than or equal to 5 pM. These antibodies also show very high affinity to cyno C3b (binding affinity less than 200 pM).

TABLE 11

| | human C3b binding | | | Cyno C3b binding | | |
|---|---|---|---|---|---|---|
| C3bneo IgG | ka (1/Ms) | kd (1/s) | K_{D} (M) | ka (1/Ms) | kd (1/s) | K_{D} (M) |
| 9556 | 3.21E+06 | 3.21E−07 | 1.00E−13 | 4.57E+06 | 4.67E−05 | 1.02E−11 |
| 9611 | 7.13E+06 | 3.10E−06 | 4.35E−13 | 6.97E+06 | 1.28E−04 | 1.83E−11 |
| 9612 | 5.05E+06 | 9.04E−06 | 1.79E−12 | 3.43E+06 | 8.67E−05 | 2.53E−11 |
| 9609 | 1.44E+06 | 3.16E−06 | 2.20E−12 | 2.02E+06 | 4.60E−05 | 2.28E−11 |
| 9610 | 8.20E+05 | 4.02E−06 | 4.90E−12 | 1.91E+06 | 6.41E−05 | 3.35E−11 |
| 9674 | 5.58E+06 | 6.66E−06 | 1.19E−12 | 8.34E+06 | 5.95E−05 | 7.13E−12 |
| 9675 | 3.53E+05 | 1.02E−06 | 2.88E−12 | 1.45E+06 | 2.90E−04 | 2.02E−10 |

SET Determination

For K_{D} determination by solution equilibrium titration (SET), monomer fractions of antibody protein were used (at least 90% monomer content, analyzed by analytical SEC; Superdex75 (Amersham Pharmacia) for Fab, or Tosoh G3000SWXL (Tosoh Bioscience) for IgG, respectively).

Affinity determination in solution was basically performed as described in the literature (Friguet et al. 305-19). In order to improve the sensitivity and accuracy of the SET method, it was transferred from classical ELISA to ECL based technology (Haenel et al., 2005).

1 mg/ml goat-anti-human (Fab)₂ fragment specific antibodies (Dianova) were labeled with MSD Sulfo-TAG™ NHS-Ester (Meso Scale Discovery, Gaithersburg, Md., USA) according to the manufacturers instructions.

The experiments were carried out in polypropylene microtiter plates and PBS pH 7.4 with 0.5% BSA and 0.02% Tween 20 as assay buffer. Unlabeled human C3b or cyno C3b was diluted in 2″ series, starting with a concentration at least 10 timer higher than the expected K_{D}. Wells without antigen were used to determine Bmax values; wells with assay buffer were used to determine background. After addition of e.g. 10 pM Fab (final concentration in 60 μL final volume), the mixture was incubated over night at RT. The applied Fab concentration was similar to or below the expected K_{D}.

Standard MSD plates were coated with 0.05 μg/ml human C3b in PBS (30 μL/well) over night and blocked with 3% BSA in PBS for 1 h. After washing the plate with assay buffer, the equilibrated samples were transferred to those plates (30 μL per well) and incubated for 20 min. After washing, 30 μL/well of the MSD-Sulfo-tag labeled detection antibody (goat anti-human (Fab)₂) in a final dilution of 1:1500 was added to the MSD plate and incubated for 30 min at RT on an Eppendorf shaker (700 rpm).

After washing the plate and adding 30 μL/well MSD Read Buffer T with surfactant, electrochemiluminescence signals were detected using a Sector Imager 6000 (Meso Scale Discovery, Gaithersburg, Md., USA).

The data was evaluated with XLfit (IDBS) software applying customized fitting models. For K_{D} determination of Fab molecules the following fit model was used (according to Haenel et al., 2005, modified according to Abraham et al., 1996):

$$y = {B}_{max} - ( \frac{{B}_{max}}{{2 [ Fab ]}_{t}} ( {[ Fab ]}_{t} + x + {K}_{D} - \sqrt{{( {[ Fab ]}_{t} + x + {K}_{D} )}^{2} - 4 {x [ Fab ]}_{t}} ) )$$

[Fab]ₜ: applied total Fab concentration
x: applied total soluble antigen concentration (binding sites)
Bₘₐₓ: maximal signal of Fab without antigen
K_{D}: affinity

In principle the same protocol was applied to determine K_{D} values of IgG molecules, with the following differences: Instead of Fab molecules, whole IgG molecules were added to the dilution series of antigen, and equilibrated over night at RT. Subsequently, the samples were treated as described above.

For data evaluation i.e. K_{D} determination of IgG molecules the following fit model for IgG was used (modified according to Piehler et al., 1997):

$$y = \frac{2 {B}_{max}}{[ IgG ]} ( \frac{[ IgG ]}{2} - \frac{{( \frac{x + [ IgG ] + {K}_{D}}{2} - \sqrt{\frac{{( x + [ IgG ] + {K}_{D} )}^{2}}{4} - x [ IgG ]} )}^{2}}{2 [ IgG ]} )$$

[IgG]: applied total IgG concentration
x: applied total soluble antigen concentration (binding sites)
Bₘₐₓ: maximal signal of IgG without antigen
K_{D}: affinity

While not specifically shown, the SET data confirms that the antibodies described herein are high affinity binders to human C3b, with binding affinities in the range of less than or equal to 10 pM, and in many cases, less than or equal to 5 pM. Similarly, the antibodies described herein bind to cynomolgus C3b with high affinity, typically with a KD in the range of less than or equal to 200 pM.

### C3b Antibodies Show Significant Binding Selectivity Against C3

The C3b antibodies were examined to determine whether they were selective for binding to C3b relative to the C3b precursor C3. Briefly, binding selectivity for C3b was determined by performing the following steps. Coat Maxisorp plate Nunc (442404) with anti-C3d rabbit monoclonal (abcam 17453) at 2 μg/ml in Carbonate Buffer at 100 μl/well. Seal plates and incubate at 4° C. overnight. Aspirate plates and wash 3 times with PBS/0.5% Tween 20. Block plates with Diluent (PBS, 4% BSA Fraction V (Fisher ICN16006980), 0.1% Tween 20 (Sigma P1379), 0.1% Triton X-100 (Sigma P234729)) and incubate for 2 h at room temperature or overnight at 4° C. Then wash plates once with PBS/0.5% Tween 20. Dilute purified C3b (complement technologies A114 lot 21) and C3 (complement technologies A113c) at 1 μg/ml in diluent and plate 100 μl per well. Incubate for 1 h at room temperature. Then wash 3 times with PBS/0.5% Tween 20.

Dilute Fabs at 100 nM and subsequent dilutions (or higher concentration) in diluent and plate 100 μl per well. Incubate at room temperature for 1 h. Wash plates 3 times with PBS/0.5% Tween 20. Add 100 μl/well of anti-Histidine-HRP monoclonal detection antibody at 1:400 in diluent and incubate at room temperature for 1 h. Then wash 4 times with PBS/0.5% Tween 20. Add 100 μl of TMB substrate (Pierce 34028) and incubate at room temperature for up to 5 min. Add 50 μl of Stop solution (2N Sulfuric Acid) to each well and read plate at 450 nm and correct for plastic reading at 570 nm.

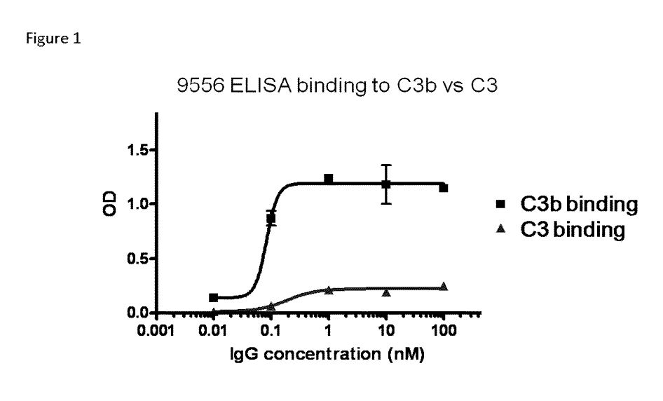

As shown in FIG. 1, the C3b antibody is selective against C3 binding. The antibody achieved more than 1000 fold C3b binding selectivity over C3 binding. While FIG. 1 shows an example of binding selectivity for antibody 9556, the 1000 fold binding selectivity is a property possessed by the seven IgG C3b antibodies disclosed herein.

### C3b Antibodies Inhibit The Alternative Complement Pathway

In order to demonstrate that the anti-C3b antibodies inhibit the alternative complement pathway, the following assays were performed.

Hemolysis Assay

The hemolytic assay is a basic functional assay that tests for complement activation and has been used to evaluate the ability of anti-human C3b mAbs and Fab molecules to block lysis of red blood cells (RBCs) by complement pathways (Evans et al. (1995). In vitro and in vivo inhibition of complement activity by a single-chain Fv fragment recognizing human C5. Mol Immunol 32, 1183-1195; Thomas et al. (1996). Inhibition of complement activity by humanized anti-05 antibody and single-chain Fv. Mol Immunol 33, 1389-1401; Rinder et al. (1995). Blockade of C5a and C5b-9 generation inhibits leukocyte and platelet activation during extracorporeal circulation. J Clin Invest 96, 1564-1572). Briefly, for classical pathway assays, sensitized red blood cells are used as targets for lysis by complement proteins present in serum. This assay is of interest for the characterization and screening of high-affinity anti-human C3b mAbs.

This procedure was adapted from Rinder et al., (1995) and Thomas et al., (1996).

### Reagents:

Rabbit red blood cells (Rb RBCs)—Lampire, Cat#7246408
Human serum—Novartis Blood Research Program; or Cyno serum—Alpha Genesis
Gelatin veronal buffer (GVB) —Boston BioProducts, Cat#IBB-300
EGTA—Boston BioProducts, Cat#BM-151
MgCl₂
U-bottom 96-well plate—Corning, Cat#3795
Flat-bottom 96-well plate—Corning, Cat#3370
NP-40—Sigma, Cat#74385

### Protocol:

1. Wash Rb RBCs and adjust to 8.33e7 cells/ml in GVB/EGTA/Mg++
2. Add 50 μl Ab diluted in GVB to wells in a 96-well round bottom plate; Ab should be at a concentration 2× of desired final concentration.
3. Add 50 μl serum diluted in GVB with EGTA and Mg++.
   a. Prepare controls wells: serum+0 nM Ab, 0% lysis control (buffer alone), 100% lysis control (0.1% NP-40), and serum, buffer, and NP-40 blanks.
   b. If testing Ab against 10% serum, use 10 mM EGTA and 5 mM Mg++ (final); if testing Ab against 50% serum, use 15-30 mM EGTA, 5 mM Mg++ (final).
4. Incubate at room temp, 30 min.
5. Add 30 μl Rb RBCs to sample and control wells; add 30 μl buffer to blank wells. Incubate 30 min at 37° C.
6. Centrifuge plate at 2000 rpm for 5 min.
7. Harvest supernatant, transfer to flat-bottom plate.
8. Read OD415 and OD570. Calculate % hemolysis:

$$\% Hemolysis = \frac{\begin{matrix}( ODsample - ODserum \cdot blank ) - \\ ( OD 0 \% lysis - ODbuffer \cdot blank )\end{matrix}}{( OD 100 \% lysis - ODNP 40 \cdot blank ) - ( OD 0 \% lysis - ODbuffer \cdot blank )}$$

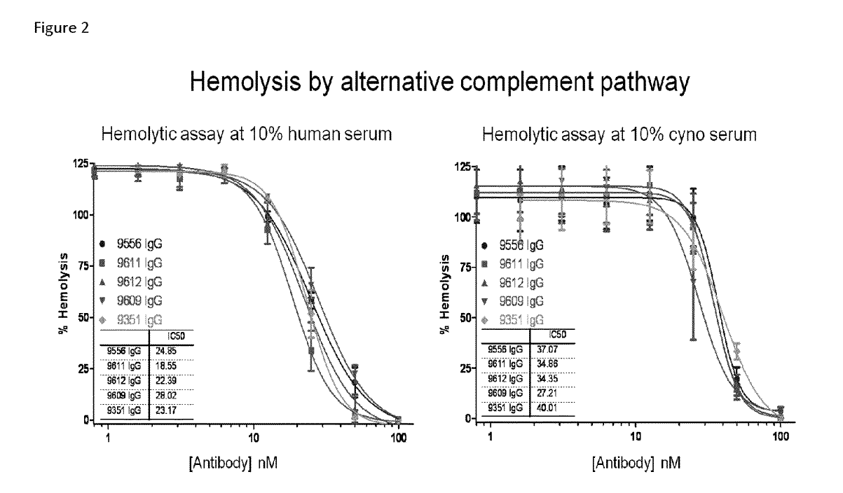

FIG. 2 shows an example of the ability of the anti-C3b antibodies to inhibit hemolysis in either 10% human or cynomolgus serum. Each of the C3b antibodies described herein inhibited hemolysis with an IC50 of less than or equal to 50 nM.

In contrast, when the assay is performed using sensitized red blood cells in order to examine activation of the classical complement pathway, the anti-C3b antibodies described herein were found not to activate the classical complement pathway (data not shown).

C3b Deposition Assay

One method of measuring the inhibitor activity against the complement C3 in the alternative pathway is to measure its breakdown product, C3b, depositing on zymosan. This ELISA based assay was performed according to the following steps: 25 μl of 1 mg/ml Zymosan A (Sigma Z4250) in carbonate buffer, pH 9.6 (Pierce Cat#28382) is coated in Maxisorp 384-well ELISA plate (Nunc 464718) overnight at 4° C. On the following day, the zymosan-coated plate is aspirated and blocked with 100 μl per well of ELISA blocking buffer, Synblock (AbD Serotec BUFO34C) for 2 h at room temperature. In a separate reaction, the inhibitors, serial diluted in gelatin veronal buffer (Boston Bioproducts IBB320-10 mM Barbital, 145 mM NaCl, 0.1% Gelatin, 0.5 mM MgCl₂, 10 mM EGTA) are added to 10% serum supplemented with MgCl₂ and EGTA for a final total reaction concentration of 1 mM MgCl₂ and 10 mM EGTA. The positive control contains no inhibitor and negative control has 25 mM EDTA. The mixture is allowed to reach equilibrium by incubating at room temperature for 30 min. To remove the blocking buffer, aspirate the plate and wash once with TBS/0.05% Tween-20. 25 μl per well of the 10% serum containing the inhibitors or controls is added to the plate and incubated at 37° C. for 30 min (previously determined by time-course to be within the linear range of C3b deposition on zymosan.) After the 30 min incubation, the plate is washed three times with TBS/0.05% Tween-20. To detect C3b deposition on zymosan, 25 μl per well of chicken anti-human C3-HRP conjugated polyclonal antibody (Immunology Consultants Laboratory, Inc. Cat#CC3-80P-1) diluted according to manufacturer in PBS with 2% BSA Fraction V (Fisher Cat#ICN 16006980), 0.1% Tween20 (Sigma Cat# P1379), and 0.1% TritonX-100 (Sigma Cat#P234729) is added to the plate and incubate at room temperature for 1 h. Afterward, wash the plate three times with TBS/0.05% Tween-20 and then add 25 μl of Ultra TMB Substrate Solution (Pierce Cat#34028.) When the solution in the well turns blue, stop the reaction with 15 μl of 2N sulfuric acid. The plate is read at 450 nm using the Spectromax with correction for the plastic plate at 570 nm (OD₄₅₀₋₅₇₀ₙₘ reading.) The percentage of C3b deposition on zymosan is calculated using the following formula:

$$\% C 3 b Deposition = 100 - 100 * \frac{[ \begin{matrix}( {OD}_{no inhibitor} - {OD}_{25 mM EDTA} ) - \\ ( {OD}_{sample} - {OD}_{25 mM EDTA} )\end{matrix} ]}{( {OD}_{no inhibitor} - {OD}_{25 mM EDTA} )}$$

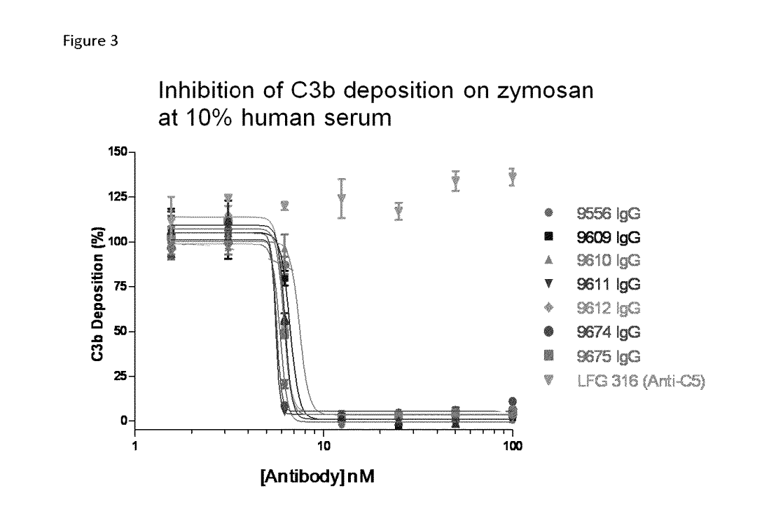

FIG. 3 shows an example of the ability of the C3b antibodies to inhibit production of C3b as a breakdown product of C3. Each of the antibodies tested were shown to inhibit C3b deposition with an IC50 of at least less than or equal to 10 nM.

MAC Deposition Assay

Another assay to determine the functional ability of the C3b antibodies to inhibit the alternative complement pathway is to measure the ability of the antibodies to inhibit the generation of the membrane attack complex (MAC), which is downstream of the processing of C3b. Briefly, Zymosan A (Sigma) was coated on a plate at 1 mg/ml in carbonate buffer, pH 9.5, to activate the Alternative Pathway. Fabs or IgGs respectively were pre-incubated with serum (2% serum, 5 mM MgCl₂, 10 mM EDTA), then added to the plate and incubated overnight at room temperature. After washing the plate 3× with TBST, MAC was detected by incubation with anti-05b-9-ALP (Diatec) for 1 h, followed by 3× washes with TBST, and incubation with 4-methylumbelliferyl phosphate (Fisher) supplemented with 2 mM MgCl₂ for 30 minutes. Reaction was stopped with 0.2M EDTA, and the plate was read at ex=355 nm, em=460 nm. Inhibition of MAC deposition was calculated for each sample relative to baseline (EDTA treated human serum) and positive control (human serum), and used to generate the IC50 curve with PRISM.

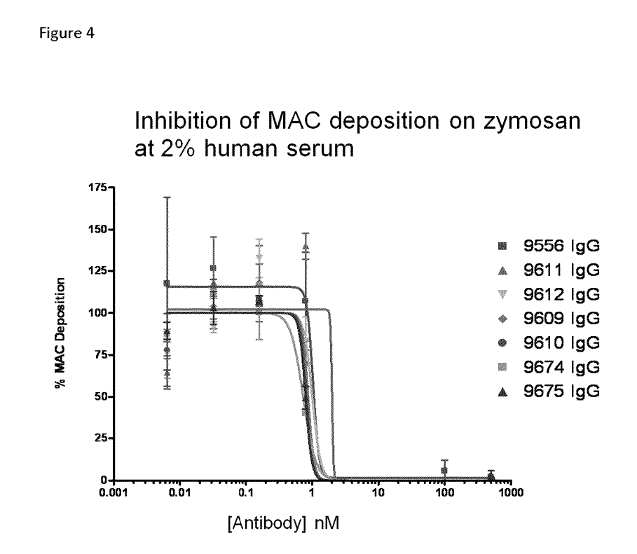

FIG. 4 shows exemplary data demonstrating the ability of the C3b antibodies to inhibit the deposition of MAC, thus indicating that the antibodies inhibit the alternative complement pathway. Specifically, the antibodies inhibited MAC deposition with an 1050 of less than or equal to 5 nM.

### Inhibition of C3a and C5a Generation

Another assay that can be used to determine the ability of a C3b antibody to inhibit the alternative complement pathway is to measure the generation of C3a and C5a, both downstream activation products of C3b in the alternative pathway.

Briefly, C5a-des-Arg ELISA was developed by Applicants to measure C5a generation during hemolysis to confirm that antibodies that were inhibitory in the hemolytic assay also inhibited cleavage of C5 into C5a and C5b.

A Maxisorp plate was coated with 100 μl/well mouse anti-human C5a-des-Arg (US Biologics) at 1 μg/ml in coating buffer (bicarbonate pH 9.5-9.8) and was incubated overnight at 4° C. After washing 3× with PBST, the plate was blocked with 300 μl/well diluent (Synblock, AbD Serotec) for 2 hours at room temperature. After aspirating the blocking solution, 100 μl samples or standards diluted with diluent were incubated for 1 hour at room temperature. Standards were prepared as follows: start was at 20 ng/ml standard (rC5a-des-Arg) and 1:4 serial dilutions were prepared for a 7-point curve. Samples of hemolytic assays were diluted 1:5 in diluent (hemolytic assay supernatants should be stored at −80° C. until used in C5a ELISA). In between the plate was washed 3× with PBST.

100 μl/well of 0.4 μg/ml detection antibody (biotin-goat anti-human c5a, R&D Systems) diluted in diluent was added and after 1 hour incubation at room temperature, 100 μl/well Strep-HRP (poly-HRP streptavidin) diluted 1:5000 in HRP diluent (poly-HRP diluent) was added for 30 minutes. After washing 4× with PBST, 100 μl/well TMB Substrate (Ultra TMB substrate solution) was added for 5-10 minutes. Reaction was stopped with 50 μl/well stop solution (2N H₂SO₄). Absorbance was read (A450-A570) and data were analyzed using SoftMax Pro.

Similarly, C3a-des-Arg ELISA was developed by Applicants to measure C3a generation during hemolysis to confirm that antibodies that were inhibitory in the hemolytic assay also inhibited cleavage of C3 into C3a and C3b.

A Maxisorp plate was coated with 100 μl/well mouse anti-human C3a-des-Arg neo (US Biologics) at 1 μg/ml in coating buffer (bicarbonate pH 9.5-9.8) and was incubated overnight at 4° C. After washing 3× with PBST, the plate was blocked with 300 μl/well diluent (Synblock, AbD Serotec) for 2 hours at room temperature. After aspirating the blocking solution, 100 μl samples or standards diluted with diluent were incubated for 1 hour at room temperature. Standards were prepared as follows: start was at 1 μg/ml standard (rC3a-des-Arg) and 1:3 serial dilutions were prepared for a 8-point curve. Samples of hemolytic assays were diluted 1:5 in diluent (hemolytic assay supernatants should be stored at −80° C. until used in C5a ELISA). In between the plate was washed 3× with PBST.

100 μl/well of 10 μg/ml detection antibody (biotin-mouse anti-human C3a, Chemicon and in-house biotinylated) diluted in diluent was added and after 1 hour incubation at room temperature, 100 μl/well Strep-HRP (poly-HRP streptavidin) diluted 1:5000 in HRP diluent (poly-HRP diluent) was added for 30 minutes. After washing 4× with PBST, 100 μl/well TMB Substrate (Ultra TMB substrate solution) was added for 5-10 minutes. Reaction was stopped with 50 μl/well stop solution (2N H₂SO₄). Absorbance was read (A450-A570) and data were analyzed using SoftMax Pro.

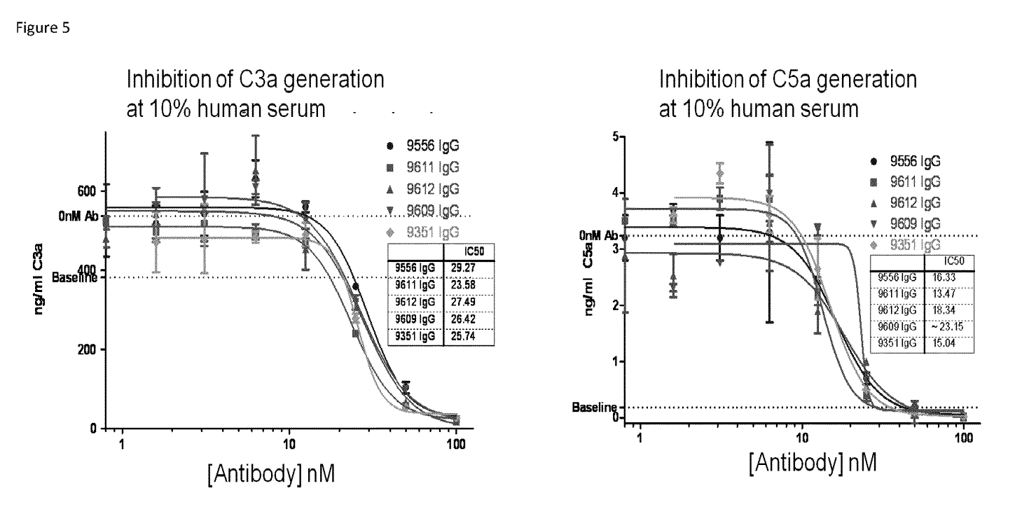

As shown in FIG. 5, the C3b antibodies were able to block the alternative pathway-driven complement activation by inhibiting the generation of C3a and C5a. More specifically, the C3b antibodies described herein inhibit the alternative pathway as measured by inhibition of C3a and C5a generation with an IC50 of less than or equal to 50 nM.

### C3b Antibodies Inhibit In Vitro C3 Convertase Enzyme Activity

C3 Water Tick-Over Convertase Gel Based Assay

In this assay, the Fabs/IgGs are pre-incubated with 10% C3-Water containing C3, such that when Factor D and Factor B are added, convertase activity via C3 water tick-over is measured. Protein Reagents are to be used at the following final concentrations: native Factor B (100 nM), Factor D (40 nM), C3 400 nM), MgCl (5 mM) and Fab/IgG 1000 nM with subsequent dilutions. In a 96-well polypropylene plate, add Fabs/IgGs at various dilutions (include PBS control sample). To this add C3 and incubate for 1 hour at room temperature. Then make a stock mixture of Factor B, Factor D, and MgCl into 1×PBS: After the 1 hour incubation, add appropriate amount of reaction mixture to the Fab/IgG-C3 in each well. Immediately take a zero time point and add 4× sample buffer and put at 95° C. Allow the convertase reaction to incubate for 15 minutes at room temperature. Then take a final time point and add 4× sample buffer and put at 95° C. Run entire volume for each sample on a 4-12% Bis-Tris Gel under reducing conditions. (Zero time points can also be generated in a separate reaction with Factor D omitted).

C3b Gel Based Convertase Assay

This assay is designed such that the Fabs/IgGs are pre-incubated with C3b. After incubation, this is added to a C3-Reaction mixture to check for convertase activity. Protein reagents are to be used at the following final concentrations: native C3b (32 nM), native Factor B (100 nM), Factor D (40 nM), C3 (400 nM), MgCl (5 mM) and Fab/IgG 1000 nM with subsequent dilutions. In a 96-well polypropylene plate, add appropriate volume of Fab/IgG at various dilutions (include PBS control sample). To this add the appropriate amount of C3b per well. Incubate for 1 hour at 37° C. Then make a stock mixture of Factor B, Factor D, and MgCl into 1×PBS. After the 1 hour incubation add C3 to the stock mixture and immediately add appropriate amount of reaction mixture to the Fab/IgG-C3 in each well. Immediately take a zero time point and add 4× sample buffer and put at 95° C. Allow the convertase reaction to incubate for 15 minutes at room temperature. Then take a final time point and add 4× sample buffer and put at 95° C. Run entire volume for each sample on a 4-12% Bis-Tris Gel run under reducing conditions. (Zero time points can also be generated in a separate reaction with Factor D omitted).

### Preformed C3 Convertase Gel Based Assay

This assay is designed such that a stable convertase is generated using C3b, Factor B mutant and Factor D. To this the Fabs/IgGs are added and allowed to incubate. Then C3 is added and samples taken for analysis. C3b is unable to form the convertase. Protein reagents are to be used at the following final concentrations: native C3b (32 nM), Factor B mutant (16 nM), Factor D (40 nM), C3 (400 nM), MgCl (5 mM) and Fab/IgG 1000 nM with subsequent dilutions. In a 96-well polypropylene plate, add cab, Factor B, Factor D and MgCl and let incubate at 37° C. for 10 minutes. Then add fabs/IgGs (PBS control) at appropriate dilutions and incubate for 20 minutes at 37° C. Then add C3 and incubate for 15 minutes at room temperature. Immediately take a zero time point and add 4× sample buffer and put at 95° C. Allow the convertase reaction to incubate for 15 minutes at room temperature. Then take a final time point and add 4× sample buffer and put at 95° C. Run entire volume for each sample on a 4-12% Bis-Tris Gel run under reducing conditions. (Zero time points can also be generated in a separate reaction with Factor D omitted).

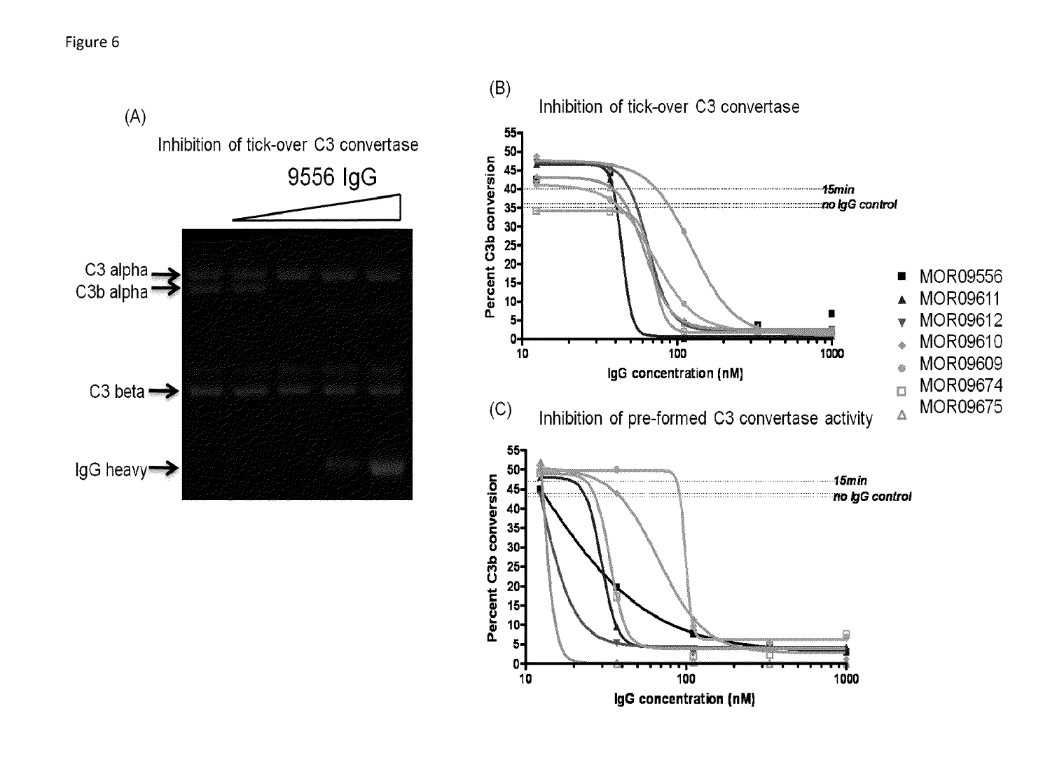

As shown in FIG. 6, C3b antibodies inhibit alternative pathway in vitro C3 convertase enzyme activities. FIG. 6A shows an SDS-PAGE gel showing the inhibition of tick-over convertase enzyme activity. FIG. 6B shows the quantitation of inhibition of C3b generation in the gel in 6A. FIG. 6C shows that anti-C3b antibodies inhibit pre-formed C3 convertase enzyme activity.

### C3b Antibodies Inhibit In Vitro C5 Convertase Enzyme Activity

In order to further characterize the ability of the anti-C3b antibodies to inhibit the alternative complement pathway, the ability of the antibodies to inhibit activation of C5 convertase was examined. Briefly, C3b was deposited on Zymosan A (Sigma) by a 10 minute incubation with purified C3 and trypsin (Sigma) at room temperature. The zymosan was centrifuged and the supernatant removed, and C3b was amplified by the addition of purified C3, fB, fD, and NiCl2. Amplification steps were repeated until desired density of C3b on the zymosan was achieved.

Fabs/IgGs were pre-incubated with Zymosan-C3b for 45 minutes at room temperature. Purified proteins were added (Complement Tech): C5 (100 nM), C6 (100 nM), fB (500 nM), fD (160 nM), and 5 mM NiCl2. The reaction was incubated at 37 C for 5 minutes, and stopped by 1:10 dilution into ice-cold GVB+10 mM EDTA. C5bC6 levels were quantified using the Hemolytic Assay, by adding the reaction product to chRBCs (8E7/ml) and 2% Human Serum, and incubating at 37 C for 30 minutes. Cells were centrifuged at 2000 rpm for 5 minutes, and the supernatant was read at A415/A570. Purified C5bC6 protein (Complement Tech) was used as a standard curve.

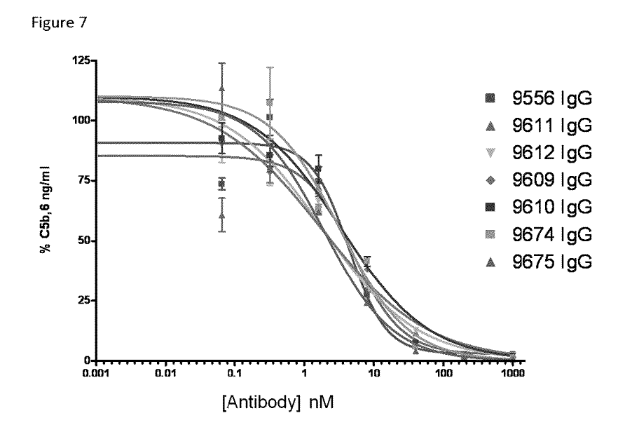

As shown in FIG. 7, the C3b antibodies inhibited alternative pathway in vitro C5 convertase enzyme activity.

### Blockade of Complement Factors Binding to C3b by C3b Antibodies

The following experiments were performed to determine the ability of anti-C3b antibodies to inhibit the interaction between C3b and other members of the alternative complement pathway and, thus, demonstrate the potential mechanisms by which the antibodies inhibit the alternative complement pathway. In brief, purified C3b and fP were conjugated to unconjugated acceptor or donor beads (PerkinElmer) using AminoLink Reductant (Pierce) during a 48 h incubation at room temperature. Beads were quenched using Carboxymethoxylamine Hemihydrochloride (Aldrich), and purified by centrifugation at 13000 rpm and three washes with 0.1M TBST. Purified fB(D24G/N260D) and fH were biotinylated during a 30 min incubation with 20-fold molar excess of NHS-Chromogenic-biotin (Pierce) at room temperature, and purified using Zeba 0.5 ml desalting columns (Pierce).

For C3b-C3b and C3b-fP proximity, Fabs or IgGs respectively were pre-incubated with C3b-Acceptor beads (20 μg/ml) for 60 min at room temperature. C3b-Donor beads or fP-Donor beads (20 μg/ml) were then added, and the plate was incubated overnight at room temperature before reading. For C3b-fB and C3b-fH proximity, antibodies were pre-incubated with C3b-Acceptor beads (20 μg/ml) for 60 minutes at room temperature. Biotinylated fB(D24G/N260D) or fH were then added, and incubated for 60 minutes at room temperature. SA-Donor beads were then added (20 μg/ml) and the plate was incubated overnight at room temperature before reading. Plates were read on the BMG Pherastar (ex=680, em=520-620).

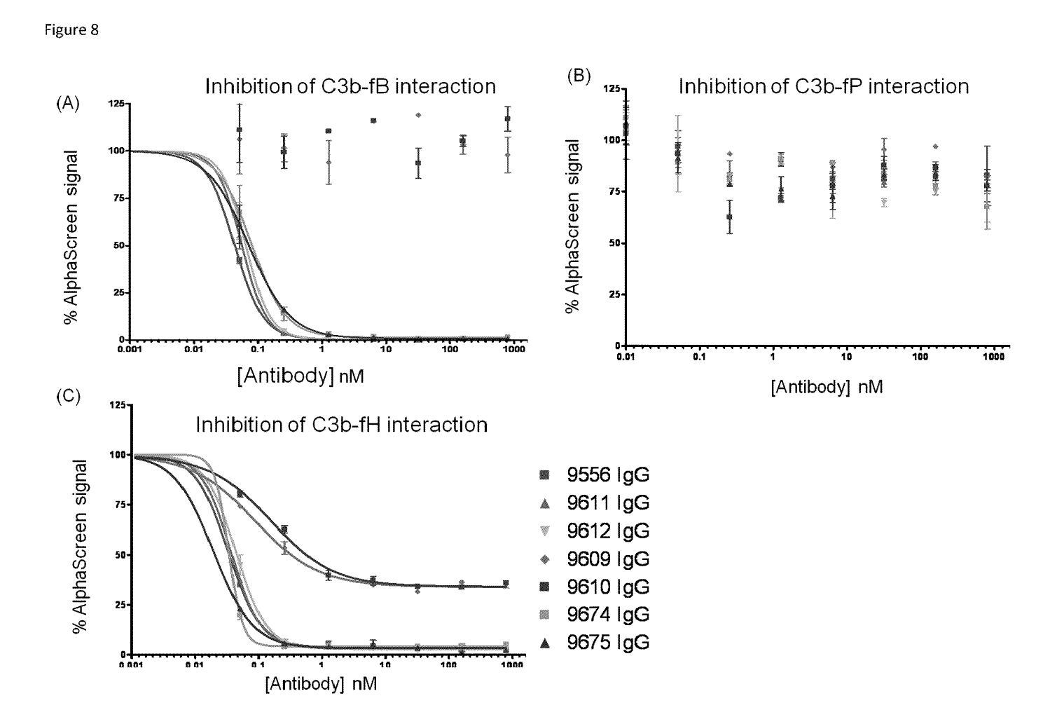

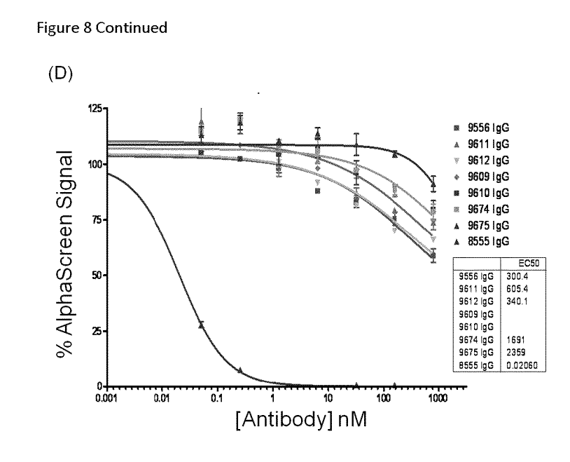

As shown in FIG. 8, C3b antibodies block the binding of several complement factors to C3b. As can be seen in the Figure, the anti-C3b antibodies utilize different mechanisms of action to block alternative complement pathway. FIG. 8A shows the inhibition of Factor B binding to C3b by C3b antibodies. FIG. 8B shows inhibition of factor P binding to C3b by C3b antibodies. FIG. 8C shows inhibition of factor H binding to C3b by C3b antibodies. FIG. 8D shows inhibition of C3b-C3b dimer formation by C3b antibodies.

C3b Antibodies do not Cross React with Human C3d or Human C5

The anti-C3b antibodies were tested for cross reactivity with human C3d and C5.

The capture antibody used in ELISAs testing for binding to human C3b, human C3d or cyno C3b is an antibody directed to the C3d protein rabbit polyclonal anti human C3d Ab, Abcam), which is a sub-domain of C3b. Wells of a Maxisorp plate were filled with the capture antibody diluted to 2 μg/ml in PBS. The plate was sealed and incubated overnight at 4° C. For testing the binding of Fabs to C5, the capture antibody was an anti-05 antibody (US Biologicals) used at a final concentration of 5 μg/ml.

On the next day remaining binding sites on the plate were blocked by adding PBST/5% milk powder. The plate was incubated for 1 h at RT and then washed 2× with PBST. C3b was added at a final concentration of 2.5 μg/ml diluted in PBST/0.5% milk powder. The plate was incubated for 1 h at RT and then washed 3× with PBST. Serial dilutions of the Fabs were made and then added to the blocked plates. The plates were incubated for 1-2 h at RT and then washed 3× with PBST.

For detection of Fabs an anti-HIS AP-conjugated antibody (Invitrogen, 46-0284) was added, the plate was incubated for 1 h at RT and then washed 5× with TBST. The fluorogenic substrate AttoPhos was used according to manufacturer's instructions. Fluorescence was measured in a Tecan GENios Pro plate reader.

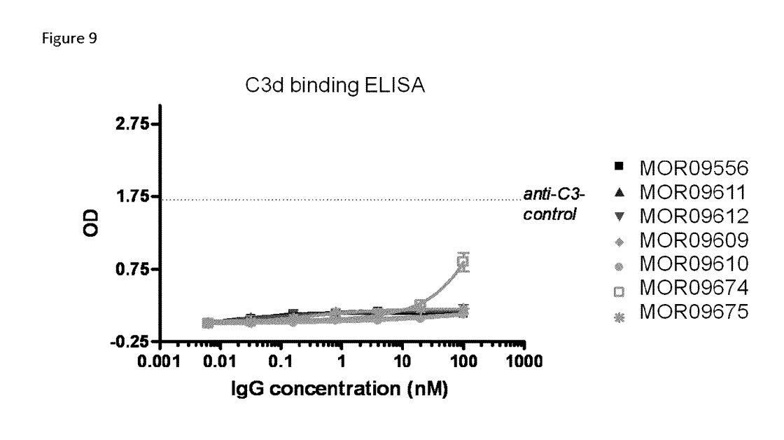

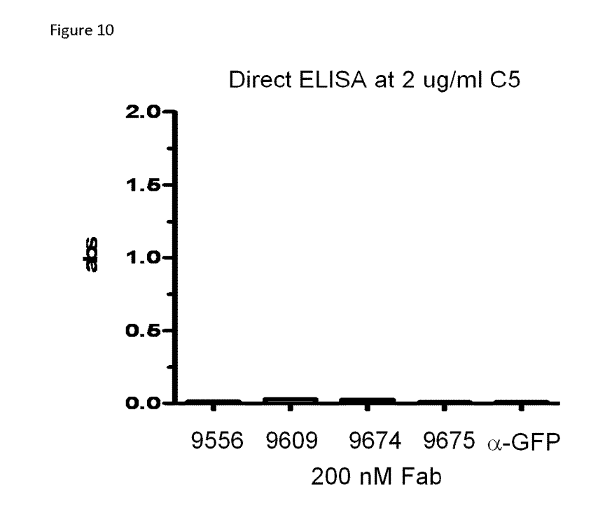

The results from these experiments for C3d and C5 are shown in FIGS. 9 and 10, respectively. As shown, the C3b antibodies do not bind either C3d or C5.

C3b Antibodies Recognize iC3b and C3c Equally Well

In order to determine whether the anti-C3b antibodies described herein can bind to complement pathway components iC3b and C3c, the following steps were performed: Directly add iC3b(complement Technologies A115), C3d (complement Technologies A117), or C3c (complement Technologies A116), at 2 μg/ml in Carbonate Buffer onto Coat Maxisorp plate Nunc (442404) at 100 μl per well. Seal plates and incubate at 4° C. overnight. Aspirate plates and wash 3 times with PBS/0.5% Tween 20. Block plates with Diluent (PBS, 4% BSA Fraction V (Fisher ICN16006980), 0.1% Tween 20 (Sigma P1379), 0.1% Triton X-100 (Sigma P234729)) and incubate for 2 h at room temperature or overnight at 4° C. Then wash plates once with PBS/0.5% Tween 20. Dilute Fabs at 100 nM and subsequent dilutions (or higher concentration if needed) in diluent and plate 100 μl per well. Incubate at room temperature for 1 h. Wash plates 3 times with PBS/0.5% Tween 20. Add 100 μl/well of anti-Histidine-HRP monoclonal detection antibody at 1:400 in diluent and incubate at room temperature for 1 h. Then wash 4 times with PBS/0.5% Tween 20. Add 100 μl of TMB substrate (Pierce 34028) and incubate at room temperature for up to 5 min. Add 50 μl of Stop solution (2N Sulfuric Acid) to each well and read plate at 450 nm and correct for plastic reading at 570 nm.

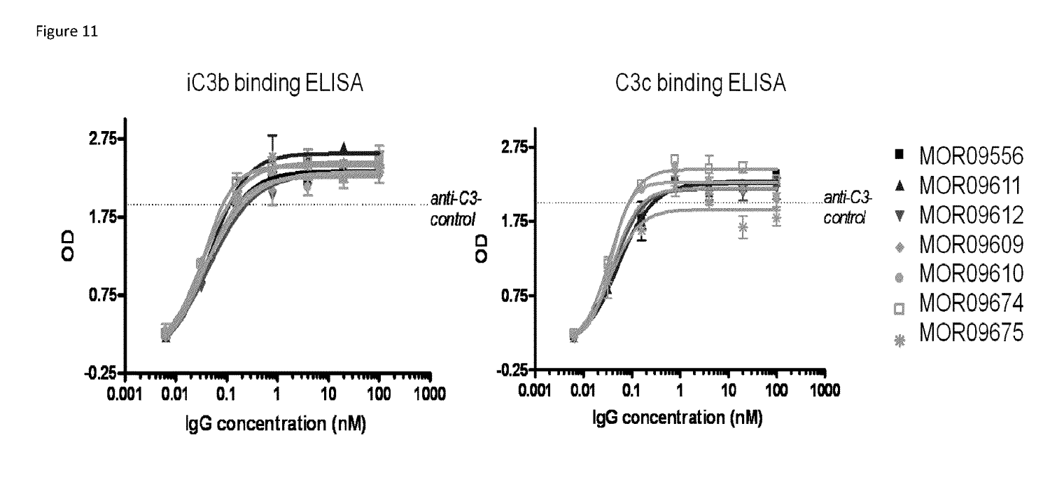

As shown in FIG. 11, the C3b antibodies cross react with both iC3b and C3c.

### Species Cross Reactivity

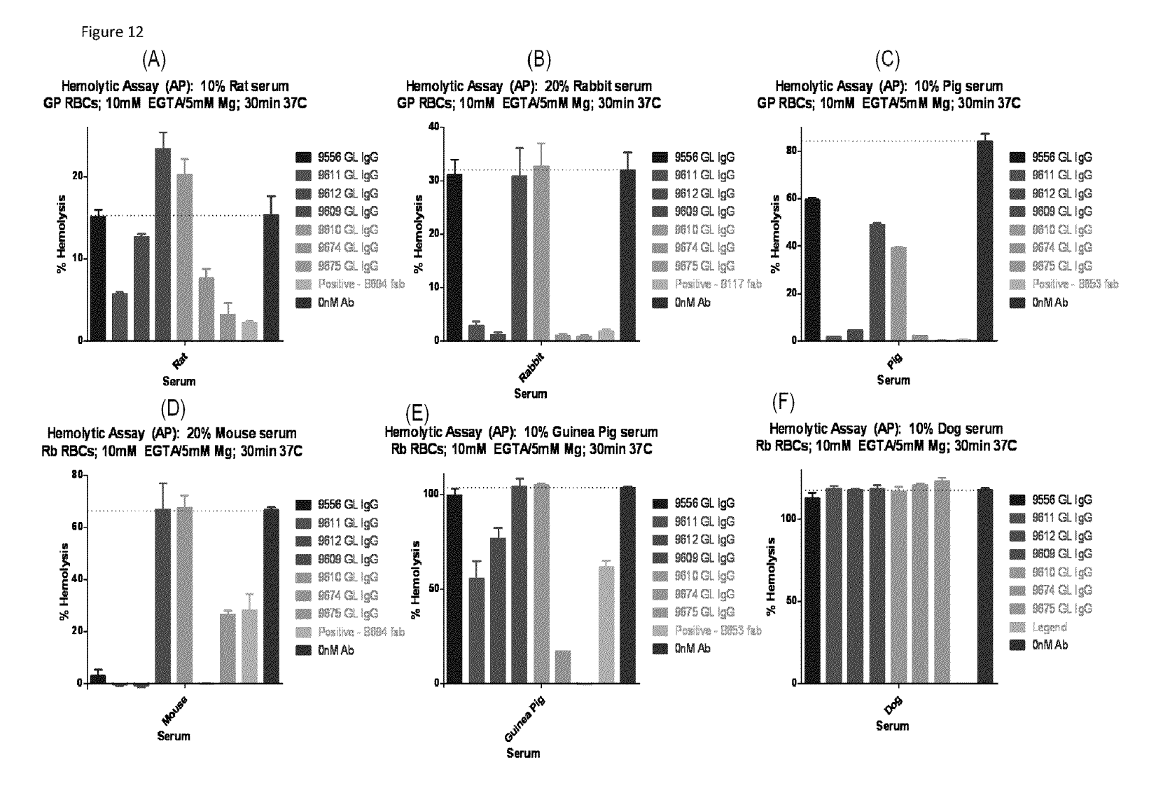

In order to determine whether, in addition to human and cynomolgus, the anti-C3b antibodies described herein would bind to C3b from other species, hemolytic assays were carried out as described above. The serum concentrations used for each species is as follows: 10% rat serum; 20% rabbit serum; 10% pig serum; 20% mouse serum; 10% guinea pig serum; and 10% dog serum. As shown in FIG. 12, the C3b antibodies were able to cross react with several species, including rat, rabbit, pig, mouse and guinea pig.

Fab Format C3bneo Binders

In addition to the full IgG format anti-C3b antibodies described above, the same variable domains were used to construct Fab format antigen binding fragments. The anti-C3b Fabs were assessed to determine their binding characteristics according to the methods described herein above. Table 11 summarizes the binding affinity, functional potency, and inhibition of complement factor binding for a subset of the Fabs.

TABLE 12

| | KD (pM), | | KD (pM), | | HA (nM), | | C3b Dep | | MAC Dep | |
|---|---|---|---|---|---|---|---|---|---|---|
| | Biacore | | SET | | 10% | | (nM) | | (nM) | |
| Fab | huC3b | cyno C3b | hu C3b | cyno C3b | human | cyno | human | cyno | human | cyno |
| MOR9556 | 6.6 | 42 | 1.5 | 18 | 62 | 99 | 24 | 43 | 4 | 18 |
| MOR9610 | 114 | 70 | 18 | 6 | 63 | 64 | 20 | 39 | 4 | 18 |
| MOR9674 | 53 | 119 | 63 | 90 | 61 | 78 | 15 | 70 | 3 | 17 |
| MOR9675 | 121 | 562 | 52 | 241 | 60 | 92 | 11 | 43 | 3 | 14 |

| | C3a (nM) | C5a (nM) | MOA IC50 (nM) | | | Cross reactivity | | | |
|---|---|---|---|---|---|---|---|---|---|
| Fab | human | human | C3b-fB | C3b-fP | C3b-fH | C5 | C3d | iC3b | C3(H2O) |
| MOR9556 | 90 | 17 | 0.15 | No | 0.2 | No | No | Yes | Yes |
| MOR9610 | 66 | 34 | No | No | No | No | No | Yes | Yes |
| MOR9674 | 81 | 31 | 2 | No | 0.2 | No | No | Yes | Yes |
| MOR9675 | 97 | 36 | 1 | No | 0.2 | No | No | Yes | Yes |

### Embodiments of the Invention

The present invention includes, but is not limited to the following embodiments:
1. An isolated antibody or antigen binding fragment thereof that specifically binds to a human or cynomolgus complement C3b protein, wherein said antibody binds to human C3b with a KD of less than or equal to 100 pM.
2. The isolated antibody or antigen binding fragment thereof of the preceding paragraph, wherein said antibody or antigen binding fragment thereof also binds to cynomolgus C3b with a KD of less than or equal to 250 pM.
3. The isolated antibody or antigen binding fragment of any preceding paragraph, wherein said antibody or antigen binding fragment thereof binds to human C3b with a KD of less than or equal to 10 pM.
4. The isolated antibody or antigen binding fragment thereof of any preceding paragraph, wherein said antibody or antigen binding fragment binds to human C3b with a KD of less than or equal to 2 pM.
5. The isolated antibody of any preceding paragraph, wherein said antibody inhibits the human alternative complement pathway as measured by an in vitro hemolytic assay with an IC50 of less than or equal to 65 nM.
6. The isolated antibody of any preceding paragraph, wherein said antibody inhibits the human alternative complement pathway as measured by in vitro C3b deposition with an IC50 of less than or equal to 50 nM.
7. The isolated antibody of any preceding paragraph, wherein said antibody inhibits the human alternative complement pathway with an IC50 of less than or equal to 5 nM as measured by deposition of the complement membrane attack complex.
8. The isolated antibody of any preceding paragraph, wherein said antibody inhibits the alternative complement pathway with an IC50 of less than or equal to 100 nM, as measured by generation of C3a and C5a
9. The isolated antibody or antigen binding fragment thereof of any preceding paragraph, wherein said antibody or antigen binding fragment thereof specifically binds to human or cynomolgus complement C3b protein, and cross competes with an antibody described in Table 1.
10. The isolated antibody of any preceding paragraph, wherein said antibody is a monoclonal antibody.
11. The isolated antibody of any preceding paragraph, wherein said antibody is a human or humanized antibody.
12. The isolated antibody of any preceding paragraph, wherein said antibody is a chimeric antibody.
13. The isolated antibody of any preceding paragraph, wherein said antibody is a single chain antibody.
14. The isolated antibody of any preceding paragraph, wherein said antibody is a Fab fragment or ScFv fragment.
15. The isolated antibody of any preceding paragraph, wherein said antibody is an IgG isotype.
16. The isolated antibody of any preceding paragraph, wherein said antibody comprises a framework in which an amino acid has been substituted into the antibody framework from the respective human VH or VL germline sequences.
17. The isolated antibody of any preceding paragraph, wherein said antibody binds to C3b with an affinity that is at least 1000 fold greater than the affinity of said antibody binding to C3.
18. The isolated antibody or antigen binding fragment thereof of any preceding paragraph, wherein said antibody or antigen binding fragment thereof comprises a heavy chain CDR1 selected from the group consisting of SEQ ID NOs 1, 15, 29, 43, 57, 71, 85, 99, 113, 127, 141, 155, 169, and 183; a heavy chain CDR2 selected from the group consisting of SEQ ID NOs: 2, 16, 30, 44, 58, 72, 86, 100, 114, 128, 142, 156, 170, and 184; and a heavy chain CDR3 selected from the group consisting of SEQ ID NOs: 3, 17, 31, 45, 59, 73, 87, 101, 115, 129, 143, 157, 171, and 185, wherein said isolated antibody or antigen binding fragment thereof binds to complement protein C3b.
19. The isolated antibody or antigen binding fragment thereof of any preceeding paragraph, wherein said antibody or antigen binding fragment comprises a light chain CDR1 selected from the group consisting of SEQ ID NOs: 4, 18, 32, 46, 60, 74, 88, 102, 116, 130, 144, 158, 172, and 186; a light chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188, wherein said isolated monoclonal antibody or antigen binding fragment thereof binds to complement protein C3b.
20. The isolated antibody or antigen binding fragment thereof of the preceding paragraph, wherein said monoclonal antibody further comprises a light chain CDR1 selected from the group consisting of SEQ ID NOs: 4, 18, 32, 46, 60, 74, 88, 102, 116, 130, 144, 158, 172, and 186; a light chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188.
21. The isolated antibody or antigen binding fragment thereof of any preceding paragraph wherein said antibody or antigen binding fragment comprises a heavy chain variable domain sequence selected from the group consisting of SEQ ID NOs: 7, 21, 35, 49, 63, 77, 91, 105, 119, 133, 147, 161, 175, and 189, and further comprises a light chain variable domain sequence selected from the group consisting of SEQ ID NOs: 8, 22, 36, 50, 64, 78, 92, 106, 120, 134, 148, 162, 176, and 190, wherein said isolated antibody or antigen binding fragment thereof binds to complement protein C3b.
22. The isolated antibody or antigen binding fragment thereof of any preceding paragraph wherein said antibody or antigen binding fragment thereof comprises a heavy chain variable domain having at least 95% sequence identity to a sequence selected from the group consisting of SEQ ID NOs: 7, 21, 35, 49, 63, 77, 91, 105, 119, 133, 147, 161, 175, and 189, wherein said antibody binds to C3b.
23. The isolated antibody or antigen binding fragment thereof of any preceding paragraph wherein said antibody or antigen binding fragment comprises a light chain variable domain having at least 95% sequence identity to a sequence selected from the group consisting of SEQ ID NOs 8, 22, 36, 50, 64, 78, 92, 106, 120, 134, 148, 162, 176, and 190, wherein said antibody binds C3b.
24. The antibody or antigen binding fragment thereof of the preceding paragraph wherein said antibody or antigen binding fragment further comprises a light chain variable domain having at least 95% sequence identity to a sequence selected from the group consisting of SEQ ID NOs 8, 22, 36, 50, 64, 78, 92, 106, 120, 134, 148, 162, 176, and 190.
25. The isolated antibody or antigen binding fragment thereof of any preceding paragraph, wherein said antibody or antigen binding fragment thereof comprises a heavy chain having at least 95% sequence identity to a sequence selected from the group consisting of SEQ ID NOs 9, 23, 37, 51, 65, 79, 93, 107, 121, 135, 149, 163, 177, and 191, wherein said antibody binds to C3b.
26. The isolated antibody or antigen binding fragment thereof of any preceding paragraph, wherein said antibody or antigen binding fragment thereof comprises a light chain having at least 95% sequence identity to a sequence selected from the group consisting of SEQ ID NOs 10, 24, 38, 52, 66, 80, 94, 108, 122, 136, 150, 164, 178, and 192, wherein said antibody binds C3b.
27. The isolated antibody or antigen binding fragment of the preceding paragraph, further comprising a light chain having at least 95% sequence identity to a sequence selected from the group consisting of SEQ ID NOs 10, 24, 38, 52, 66, 80, 94, 108, 122, 136, 150, 164, 178, and 192.
28. A pharmaceutical composition comprising the antibody or antigen binding fragment thereof of any preceding paragraph and a pharmaceutically acceptable carrier.
29. An isolated nucleic acid comprising a sequence encoding a polypeptide comprising a heavy chain variable domain having at least 95% sequence identity to a sequence selected from the group consisting of SEQ ID NOs: 7, 21, 35, 49, 63, 77, 91, 105, 119, 133, 147, 161, 175, and 189.
30. An isolated nucleic acid comprising a sequence encoding a polypeptide comprising a light chain variable domain having at least 95% sequence identity to a sequence selected from the group consisting of SEQ ID NOs 8, 22, 36, 50, 64, 78, 92, 106, 120, 134, 148, 162, 176, and 190.
31. A vector comprising the nucleic acid of the preceding paragraphs.
32. An isolated host cell comprising a recombinant DNA sequence encoding a heavy chain of the antibody or antigen binding fragment thereof of any preceding paragraph, and a second recombinant DNA sequence encoding a light chain of the antibody or antigen binding fragment thereof of any preceding paragraph, wherein said DNA sequences are operably linked to a promoter and are capable of being expressed in the host cell.
33. The isolated host cell of the preceding paragraph, wherein said antibody is a human monoclonal antibody.
34. The isolated host cell of the preceding two paragraphs, wherein said host cell is a non-human mammalian cell.
35. A method of treating age related macular degeneration comprising administering to a subject in need thereof an effective amount of a composition comprising the antibody or antigen binding fragment thereof of the preceding paragraphs.
36. The method of the preceding paragraph, wherein said subject is human.
37. A method of inhibiting the alternative complement pathway in a subject comprising administering to a subject in need thereof, an effective amount of a composition comprising the antibody or antigen binding fragment thereof of the preceding paragraphs.
38. The method of the preceding paragraph, wherein said subject is human.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. An isolated antibody or antigen binding fragment thereof that specifically binds to a human or cynomolgus complement C3b protein, wherein said antibody binds to human C3b with a KD of less than or equal to 100 pM.

2. The isolated antibody or antigen binding fragment thereof of claim 1, wherein said antibody or antigen binding fragment thereof also binds to cynomolgus C3b with a KD of less than or equal to 250 pM.

3. The isolated antibody or antigen binding fragment of claim 1, wherein said antibody or antigen binding fragment thereof binds to human C3b with a KD of less than or equal to 10 pM.

4. The isolated antibody or antigen binding fragment thereof of claim 1, wherein said antibody or antigen binding fragment binds to human C3b with a KD of less than or equal to 2 pM.

5. The isolated antibody of claim 1, wherein said antibody inhibits the human alternative complement pathway as measured by an in vitro hemolytic assay with an IC50 of less than or equal to 65 nM.

6. The isolated antibody of claim 1, wherein said antibody inhibits the human alternative complement pathway as measured by in vitro C3b deposition with an IC50 of less than or equal to 50 nM.

7. The isolated antibody of claim 1, wherein said antibody inhibits the human alternative complement pathway with an IC50 of less than or equal to 5 nM as measured by deposition of the complement membrane attack complex.

8. The isolated antibody of claim 1, wherein said antibody inhibits the alternative complement pathway with an IC50 of less than or equal to 100 nM, as measured by generation of C3a and C5a

9. The isolated antibody or antigen binding fragment thereof of claim 1, wherein said antibody or antigen binding fragment thereof specifically binds to human or cynomolgus complement C3b protein, and cross competes with an antibody described in Table 1.

10. The isolated antibody of claim 1, wherein said antibody is a monoclonal antibody.

11. The isolated antibody of claim 1, wherein said antibody is a human or humanized antibody.

12. The isolated antibody of claim 1, wherein said antibody is a chimeric antibody.

13. The isolated antibody of claim 1, wherein said antibody is a single chain antibody.

14. The isolated antibody of claim 1, wherein said antibody is a Fab fragment or ScFv fragment.

15. The isolated antibody of claim 1, wherein said antibody is an IgG isotype.

16. The isolated antibody of claim 1, wherein said antibody comprises a framework in which an amino acid has been substituted into the antibody framework from the respective human VH or VL germline sequences.

17. The isolated antibody of claim 1, wherein said antibody binds to C3b with an affinity that is at least 1000 fold greater than the affinity of said antibody binding to C3.

18. The isolated antibody or antigen binding fragment thereof of claim 1, wherein said antibody or antigen binding fragment thereof comprises a heavy chain CDR1 selected from the group consisting of SEQ ID NOs 1, 15, 29, 43, 57, 71, 85, 99, 113, 127, 141, 155, 169, and 183; a heavy chain CDR2 selected from the group consisting of SEQ ID NOs: 2, 16, 30, 44, 58, 72, 86, 100, 114, 128, 142, 156, 170, and 184; and a heavy chain CDR3 selected from the group consisting of SEQ ID NOs: 3, 17, 31, 45, 59, 73, 87, 101, 115, 129, 143, 157, 171, and 185, wherein said isolated antibody or antigen binding fragment thereof binds to complement protein C3b.

19. The isolated antibody or antigen binding fragment thereof of claim 1, wherein said antibody or antigen binding fragment comprises a light chain CDR1 selected from the group consisting of SEQ ID NOs: 4, 18, 32, 46, 60, 74, 88, 102, 116, 130, 144, 158, 172, and 186; a light chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188, wherein said isolated monoclonal antibody or antigen binding fragment thereof binds to complement protein C3b.

20. The isolated antibody or antigen binding fragment thereof of claim 18, wherein said monoclonal antibody further comprises a light chain CDR1 selected from the group consisting of SEQ ID NOs: 4, 18, 32, 46, 60, 74, 88, 102, 116, 130, 144, 158, 172, and 186; a light chain CDR2 selected from the group consisting of SEQ ID NOs 5, 19, 33, 47, 61, 75, 89, 103, 117, 131, 145, 159, 173, and 187; and a light chain CDR3 selected from the group consisting of SEQ ID NOs 6, 20, 34, 48, 62, 76, 90, 104, 118, 132, 146, 160, 174, and 188.

21. The isolated antibody or antigen binding fragment thereof of claim 1 wherein said antibody or antigen binding fragment comprises a heavy chain variable domain sequence selected from the group consisting of SEQ ID NOs: 7, 21, 35, 49, 63, 77, 91, 105, 119, 133, 147, 161, 175, and 189, and further comprises a light chain variable domain sequence selected from the group consisting of SEQ ID NOs: 8, 22, 36, 50, 64, 78, 92, 106, 120, 134, 148, 162, 176, and 190, wherein said isolated antibody or antigen binding fragment thereof binds to complement protein C3b.

22. The isolated antibody or antigen binding fragment thereof of claim 1 wherein said antibody or antigen binding fragment thereof comprises a heavy chain variable domain having at least 95% sequence identity to a sequence selected from the group consisting of SEQ ID NOs: 7, 21, 35, 49, 63, 77, 91, 105, 119, 133, 147, 161, 175, and 189, wherein said antibody binds to C3b.

23. The isolated antibody or antigen binding fragment thereof of claim 1 wherein said antibody or antigen binding fragment comprises a light chain variable domain having at least 95% sequence identity to a sequence selected from the group consisting of SEQ ID NOs 8, 22, 36, 50, 64, 78, 92, 106, 120, 134, 148, 162, 176, and 190, wherein said antibody binds C3b.

24. The antibody or antigen binding fragment thereof of claim 22 wherein said antibody or antigen binding fragment further comprises a light chain variable domain having at least 95% sequence identity to a sequence selected from the group consisting of SEQ ID NOs 8, 22, 36, 50, 64, 78, 92, 106, 120, 134, 148, 162, 176, and 190.

25. The isolated antibody or antigen binding fragment thereof of claim 1, wherein said antibody or antigen binding fragment thereof comprises a heavy chain having at least 95% sequence identity to a sequence selected from the group consisting of SEQ ID NOs 9, 23, 37, 51, 65, 79, 93, 107, 121, 135, 149, 163, 177, and 191, wherein said antibody binds to C3b.

26. The isolated antibody or antigen binding fragment thereof of claim 1, wherein said antibody or antigen binding fragment thereof comprises a light chain having at least 95% sequence identity to a sequence selected from the group consisting of SEQ ID NOs 10, 24, 38, 52, 66, 80, 94, 108, 122, 136, 150, 164, 178, and 192, wherein said antibody binds C3b.

27. The isolated antibody or antigen binding fragment of claim 25, further comprising a light chain having at least 95% sequence identity to a sequence selected from the group consisting of SEQ ID NOs 10, 24, 38, 52, 66, 80, 94, 108, 122, 136, 150, 164, 178, and 192.

28. A pharmaceutical composition comprising the antibody or antigen binding fragment thereof of claim 1 and a pharmaceutically acceptable carrier.

29. An isolated nucleic acid comprising a sequence encoding a polypeptide comprising a heavy chain variable domain having at least 95% sequence identity to a sequence selected from the group consisting of SEQ ID NOs: 7, 21, 35, 49, 63, 77, 91, 105, 119, 133, 147, 161, 175, and 189.

30. An isolated nucleic acid comprising a sequence encoding a polypeptide comprising a light chain variable domain having at least 95% sequence identity to a sequence selected from the group consisting of SEQ ID NOs 8, 22, 36, 50, 64, 78, 92, 106, 120, 134, 148, 162, 176, and 190.

31. A vector comprising the nucleic acid of claim 29 or 30.

32. An isolated host cell comprising a recombinant DNA sequence encoding a heavy chain of the antibody or antigen binding fragment thereof of claim 1, and a second recombinant DNA sequence encoding a light chain of the antibody or antigen binding fragment thereof of claim 1, wherein said DNA sequences are operably linked to a promoter and are capable of being expressed in the host cell.

33. The isolated host cell of claim 32, wherein said antibody is a human monoclonal antibody.

34. The isolated host cell of claim 32, wherein said host cell is a non-human mammalian cell.

35. A method of treating age related macular degeneration comprising administering to a subject in need thereof an effective amount of a composition comprising the antibody or antigen binding fragment thereof of claim 1.

36. The method of claim 35, wherein said subject is human.

37. A method of inhibiting the alternative complement pathway in a subject comprising administering to a subject in need thereof, an effective amount of a composition comprising the antibody or antigen binding fragment thereof of claim 1.

38. The method of claim 37, wherein said subject is human.
